(19) United States
(12) Patent Application Publication
(10) Pub. No.: US 2023/0270145 A1
(43) Pub. Date: Aug. 31, 2023

(54) PRODUCTS FOR ORAL CONSUMPTION WITH REDUCED SUGAR CONTENT

(71) Applicant: Nomad Bioscience GmbH, München (DE)

(72) Inventors: Anett Stephan, Halle (DE); Simone Hahn-Löbmann, Merseburg (DE); Heike Prochaska, Halle (Saale) (DE); Anatoli Giritch, Halle (DE); Yuri Gleba, Berlin (DE)

(21) Appl. No.: 18/016,051

(22) PCT Filed: Jun. 29, 2021
(86) PCT No.: PCT/EP2021/067900

§ 371 Date: Jan. 13, 2023

(30) Foreign Application Priority Data
Jul. 16, 2020 (EP) 20186323.0

(51) Int. Cl. A23L 27/30; A23L 2/60

(52) U.S. Cl. CPC A23L 27/31; A23L 27/33; A23L 2/60

(57) ABSTRACT

A product for oral consumption, such as a food, comprising at least one thaumatin selected from the group consisting of thaumatin I and thaumatin II and at least one sugar selected from the group consisting of sucrose, glucose, and fructose in a mass ratio of said thaumatin:to said sugar of from 1:2,000 to 1:80,000.

### FIELD OF THE INVENTION

The present invention relates to a product for oral consumption, such as a food, comprising thaumatin and at least one sugar selected from the group consisting of sucrose, glucose, and fructose. In the product, the content of sugar can be reduced with little effect on the sweetness and other taste properties. Further, the present invention relates to a composition suitable for producing a product for oral consumption, the composition comprising at least one thaumatin and at least one sugar selected from the group consisting of sucrose, glucose, and fructose. Uses of such composition as a sweetening composition or for reducing the caloric content of a product for oral consumption are provided. The present invention also provides methods of reducing the content of sugars in products for oral consumption and a method of reducing the dry taste of High Fructose Corn Syrup in a product for oral consumption.

### BACKGROUND OF THE INVENTION

Excessive consumption of sugar-containing foods and beverages has become an increasing problem for the health for many people in the developed as well as in the developing world. Health problems arising therefrom are notorious and include obesity, type II diabetes, insulin resistance, metabolic syndrome, as well as health problems and risks derived therefrom, such as cardiovascular diseases and even increased vulnerability to infectious diseases such as Covid-19.

Recent trends in the food industry require producers to reduce the content of sugar and/or High Fructose Corn Syrup (HFCS) and, thus, caloric content. However, the taste properties of the products should not be changed too much in order not to compromise recognizability of products by consumers. As the sugar content has a dominant effect not only on taste, notably sweetness perception, but also on other organoleptic properties, such as mouth feel, viscosity, and texture, as well as on preservation against microbial attack, it is not an easy task to reduce the sugar content of sugar-containing products, notably products high in sugar content, without compromising other desired properties of the products.

Therefore, it is an object of the present invention to provide a product and composition for oral consumption, such as food, that are reduced in the content of sugar. It is another object of the invention to provide products and compositions for oral consumption that are reduced in sugar content, and achieve the same or highly similar taste properties with respect to sweetness, overall taste and aftertaste as conventional products. It is a further object to provide sweetening compositions for products for oral consumption, and methods of reducing the sugar content in a product for oral consumption with little or no change of taste properties like sweetness, overall taste and aftertaste.

### SUMMARY OF THE INVENTION

These objects are accomplished by:
1) A product for oral consumption, such as a food, comprising at least one thaumatin selected from the group consisting of thaumatin I and thaumatin II and at least one sugar selected from the group consisting of sucrose, glucose, and fructose in a mass ratio of said thaumatin:to said sugar of
   from 1:2,000 to 1:80,000,
   preferably from 1:4,000 to 1:65,000,
   more preferably from 1:6,000 to 1:55,000,
   even more preferably from 1:8,000 to 1:45,000, and
   even more preferably from 1:10,000 to 1:35,000.
2) A product for oral consumption comprising thaumatin in a range from 1 to 13 ppm, preferably from 3 ppm to 7 ppm, per total weight of the product and at least one sugar selected from the group consisting of sucrose, glucose, and fructose, wherein the thaumatin is at least one thaumatin selected from the group consisting of thaumatin I and thaumatin II.
3) The product for oral consumption according to item 1 or 2, comprising, with respect to the total weight of the product, 2 to 12 weight %, preferably from 3 to 10 weight %, more preferably from 4 to 8 weight % of said sugar selected from the group consisting of sucrose, glucose, and fructose.
4) The product for oral consumption according to any one of items 1 to 3, wherein said at least one sugar is selected from the group consisting glucose and fructose, said product not containing sucrose.
5) The product for oral consumption according to any one of items 1 to 4, said product comprising High Fructose Corn Syrup (HFCS) containing at least one sugar selected from the group consisting of glucose and fructose.
6) The product for oral consumption according to item 5, wherein the HFCS is HFCS-42, HFCS-55, HFCS-65, HFCS-70 or HFCS-90.
7) The product for oral consumption according item 5 or 6, wherein the HFCS contains, per total weight of the HFCS, 22 to 25 weight % water and 78% to 75% dissolved or dispersed solids,
   wherein said solids contain 15 to 92 weight % fructose, 8 to 85 weight % glucose, and 0 to 7 weight % glucose oligosaccharides per total weight of the solids, preferably said solids contain 40 to 65 weight % fructose, 30 to 55 weight % glucose, and 0 to 7 weight % glucose oligosaccharides per total weight of the solids.
8) A product for oral consumption, such as a food, comprising at least one thaumatin selected from the group consisting of thaumatin I and thaumatin II and a sugar selected from the group consisting of glucose and fructose, said product comprising the sugar selected from the group consisting of glucose and fructose in a mass ratio of said thaumatin:to said sugar of
from 1:2,000 to 1:80,000,
preferably from 1:4,000 to 1:65,000,
more preferably from 1:6,000 to 1:55,000,
even more preferably from 1:8,000 to 1:45,000, an
even more preferably from 1:10,000 to 1:35,000.
9) The product for oral consumption according to any one of items 1 to 8, said product comprising High Fructose Corn Syrup (HFCS) as said at least one sugar selected from the group consisting of glucose and fructose.
10) The product for oral consumption according to any one of items 1 to 9, wherein the product is a beverage, beverage powder, drink, soft drink, yoghurt, jam, marmalade, beverage concentrate such as syrup, dessert, cake, biscuit, cookie, chocolate, candy, sweets, sugar confectionary, chewing gum, custard, pudding, jelly, filling jelly, pastry, pie, drops, processed foods, cereals, baked goods, or a medicament; wine or beer or other fermented or distilled beverages, potato-based snacks, breakfast cereals, chewing gum, ice cream, cocoa and chocolate products, breath mints, sugar decorations or icings, coatings or fillings, fine bakery items, food supplements or table-top sweeteners.
11) A composition suitable for producing a product for oral consumption, the composition comprising at least one thaumatin selected from the group consisting of thaumatin I and thaumatin II and at least one sugar selected from the group consisting of sucrose, glucose, and fructose.
12) The composition according to item 11, containing at least one sugar selected from the group consisting of glucose and fructose, said composition not containing sucrose.
13) A composition suitable for producing a product for oral consumption, the composition comprising at least one thaumatin selected from the group consisting of thaumatin I and thaumatin II and High Fructose Corn Syrup (HFCS) comprising at least one sugar selected from the group consisting of glucose and fructose.
14) The composition according to any one of items 11 to 13, containing said at least one thaumatin in a range of from 1 to 13 ppm per total weight of the composition, preferably from 3 ppm to 7 ppm per total weight of the composition.
15) The composition according to any one of items 11 to 14, containing said at least one thaumatin and said at least one sugar in a mass ratio of thaumatin:sugar of
   from 1:2,000 to 1:80,000,
   preferably from 1:4,000 to 1:65,000,
   more preferably from 1:6,000 to 1:55,000,
   even more preferably from 1:8,000 to 1:45,000, and
   even more preferably from 1:10,000 to 1:35,000.
16) The composition according to any one of items 11 to 15, comprising said at least one thaumatin in a range of from 10 to 100 ppm per total weight of the composition and said at least one sugar in a range from 30 to 99.9%, preferably from 40 to 98%, more preferably from 50 to 90%, and even more preferably from 60 to 80% by weight per total weight of the composition.
17) The composition according to any one of items 11 to 15, comprising said at least one thaumatin in a range from 30 to 70 ppm per total weight of the composition and said at least one sugar in a range from 30 to 99.9%, preferably from 40 to 98%, more preferably from 50 to 90%, and even more preferably from 60 to 80% by weight per total weight of the composition.
18) The composition according to any one of items 11 to 17, comprising a further component for said product for oral consumption, said component being one or more component(s) selected from the group consisting of citric acid or a salt thereof, a vitamin, an inorganic salt, a trace element, caffeine, taurine, a gelling or thickening agent, a flavoring agent, and a preservative.
19) The composition according to item 18, wherein said vitamin is one or more selected from ascorbic acid or a salt thereof, a vitamin B family vitamin, or tocopherol or a derivative thereof, said inorganic salt is selected from a sodium salt, a magnesium salt, a potassium salt, and a calcium salt; and/or said trace element is a zinc compound, an iron compound, or a copper compound.
20) The composition according to item 13, wherein the HFCS is HFCS-42, HFCS-55, HFCS-65, HFCS-70 or HFCS-90.
21) The composition according to item 13 or 20, wherein the HFCS contains, per total weight of the HFCS, 22 to 25 weight % water and 78 to 75 weight % dissolved or dispersed solids,
   wherein said solids contain 15 to 92 weight % fructose, 8 to 85 weight % glucose, and 0 to 7 weight % glucose oligosaccharides per total weight of the solids, preferably said solids contain 40 to 65 weight % fructose, 30 to 55 weight % glucose, and 0 to 7 weight % glucose oligosaccharides per total weight of the solids.
22) The product or composition according to any one of items 1 to 21, wherein said thaumatin is thaumatin II.
23) A use of a composition as defined in any one of items 11 to 22 as a sweetening composition.
24) A use of a composition as defined in any one of items 11 to 22 for preparing a product for oral consumption.
25) A use of a thaumatin selected from the group consisting of thaumatin I and thaumatin II, preferably of thaumatin II, to reduce the caloric content of a product for oral consumption.
26) A method of reducing the content of sugar in a product for oral consumption, the sugar being at least one selected from the group consisting of sucrose, glucose, and fructose, the method comprising replacing a part of said sugar with thaumatin in a range from 1 ppm to 13 ppm per total weight of the obtained product, preferably from 3 ppm to 7 ppm, wherein the thaumatin is selected from the group consisting of thaumatin I and thaumatin II.
27) The method according to item 26, wherein the content of the at least one sugar selected from the group consisting of sucrose, glucose, and fructose is reduced by up to 50% in a product for oral consumption, the method comprising replacing a part of said sugar with thaumatin in a range from 1 ppm to 13 ppm per total weight of the obtained product, preferably from 3 ppm to 7 ppm, wherein the thaumatin is selected from the group consisting of thaumatin I and thaumatin II.
28) A method of reducing the content of sugar in a product for oral consumption by up to 50%, the sugar being at least one selected from the group consisting of sucrose, glucose, and fructose, the method comprising replacing a part of said sugar with thaumatin in a range from 1 ppm to 13 ppm per total weight of the obtained product, preferably from 3 ppm to 7 ppm, wherein the thaumatin is selected from the group consisting of thaumatin I and thaumatin II.
29) A method of using thaumatin as a sweetener in a product for oral consumption, the method comprising adding thaumatin to a precursor of said product in a range from 1 ppm to 13 ppm per total weight of the product, wherein the thaumatin is selected from the group consisting of thaumatin I and thaumatin II.
30) A method of producing a product for oral consumption, comprising mixing the composition according to any one of items 11 to 22 with other components to produce said product.
31) A method of reducing the dry taste of a HFCS-containing product for oral consumption, the method comprising adding a thaumatin to a pre-product of said HFCS-containing product, said pre-product having a reduced content of HFCS, wherein the thaumatin is selected from the group consisting of thaumatin I and thaumatin II.
32) A method of reducing the dry taste of High Fructose Corn Syrup (HFCS) in a product for oral consumption, the method comprising replacing a part of the HFCS with thaumatin, wherein the content of the solid content of HFCS in the obtained product is reduced to 4-8% by weight, preferably to 5-7% by weight, per total weight of the obtained product and replaced with thaumatin in a range from 2 ppm to 3.5 ppm per total weight of the obtained product, wherein the thaumatin is selected from the group consisting of thaumatin I and thaumatin II.
33) A method of reducing the dry taste of High Fructose Corn Syrup (HFCS) in a product for oral consumption, the method comprising replacing a part of the HFCS with thaumatin, wherein the content of the HFCS solids in the obtained product is reduced by 30-50% and is replaced with thaumatin in a range from 2 ppm to 3.5 ppm per total weight of the obtained product, wherein the thaumatin is selected from the group consisting of thaumatin I and thaumatin II.
34) Use of thaumatin for reducing the dry taste of High Fructose Corn Syrup (HFCS) in a product for oral consumption by reducing the content HFCS solids in the product to 4-8% by weight, preferably 5-7% by weight, per total weight of the obtained product and adding thaumatin in a range from 1 ppm to 4.5 ppm per total weight of the obtained product, wherein the thaumatin is selected from the group consisting of thaumatin I and thaumatin II.
35) The method according to any one of items 31 to 33 or the use according to item 34, wherein the High Fructose Corn Syrup (HFCS) is HFCS-55.
36) A product for oral consumption containing 5-7% by weight HFCS solids per total weight of the product and thaumatin in a range from 1 ppm to 4.5 ppm per total weight of the product, wherein the HFCS solids are the solid content of High Fructose Corn Syrup and the thaumatin is selected from the group consisting of thaumatin I and thaumatin II.
37) The product, the composition, the use or the method according to any one of items 1 to 36, wherein said thaumatin is a protein comprising a polypeptide the amino acid sequence of which is that of SEQ ID NO: 5 or SEQ ID NO: 6, or an amino acid sequence having from 1 to 3 amino acid substitutions, additions, deletions, and/or insertions in the amino acid sequence of SEQ ID NO: 5 or 6, said thaumatin is preferably extracted from Nicotiana-species.
38) Plant extract comprising thaumatin selected from thaumatin I and thaumatin II and at least one sugar selected from the group consisting of sucrose, glucose, and fructose
39) A product for oral consumption, such as a food, comprising at least one thaumatin selected from the group consisting of thaumatin I and thaumatin II and at least one sugar selected from the group consisting of sucrose, glucose, and fructose in a mass ratio of said thaumatin:to said sugar of
from 1:2,000 to 1:80,000,
preferably from 1:4,000 to 1:65,000,
more preferably from 1:6,000 to 1:55,000,
even more preferably from 1:8,000 to 1:45,000, and
even more preferably from 1:10,000 to 1:35,000.
40) The product for oral consumption according to item 39, comprising, with respect to the total weight of the product, 2 to 12 weight %, preferably from 3 to 10 weight %, more preferably from 4 to 8 weight % of said sugar selected from the group consisting of sucrose, glucose, and fructose; and/or
   comprising thaumatin in a range from 1 to 13 ppm per total weight of the product, preferably from 3 ppm to 7 ppm, wherein the thaumatin is at least one thaumatin selected from the group consisting of thaumatin I and thaumatin II.
41) The product for oral consumption according to item 39 or 40, wherein said at least one sugar is selected from the group consisting glucose and fructose, said product not containing sucrose.
42) The product for oral consumption according to any one of items 39 to 41, said product comprising High Fructose Corn Syrup (HFCS) containing at least one sugar selected from the group consisting of glucose and fructose, preferably the HFCS is HFCS-42, HFCS-55, HFCS-65, HFCS-70 or HFCS-90.
43) The product for oral consumption according to item 42, wherein the HFCS contains, per total weight of the HFCS, 22 to 25 weight % water and 78% to 75% dissolved or dispersed solids,
   wherein said solids contain 15 to 92 weight % fructose, 8 to 85 weight % glucose, and 0% to 7% glucose oligosaccharides per total weight of the solids, preferably said solids contain 40 to 65 weight % fructose, 30 to 55 weight % glucose, and 0% to 7% glucose oligosaccharides per total weight of the solids.
44) The product for oral consumption according to any one of items 39 to 43, wherein the product is a beverage, beverage powder, drink, soft drink, yoghurt, jam, marmalade, beverage concentrate such as syrup, dessert, cake, biscuit, cookie, chocolate, candy, sweets, sugar confectionary, chewing gum, custard, pudding, jelly, filling jelly, pastry, pie, drops, processed foods, cereals, baked goods, or a medicament; wine or beer or other fermented or distilled beverages, potato-based snacks, breakfast cereals, chewing gum, ice cream, cocoa and chocolate products, breath mints, sugar decorations or icings, coatings or fillings, fine bakery items, food supplements or table-top sweeteners.
45) A composition suitable for producing a product for oral consumption, the composition comprising at least one thaumatin selected from the group consisting of thaumatin I and thaumatin II and at least one sugar selected from the group consisting of sucrose, glucose, and fructose; or
   the composition comprising at least one thaumatin selected from the group consisting of thaumatin I and thaumatin II and High Fructose Corn Syrup (HFCS) comprising at least one sugar selected from glucose and fructose.
46) The composition according to item 45, containing said at least one thaumatin in a range from 1 to 13 ppm per total weight of the composition, preferably from 3 ppm to 7 ppm per total weight of the composition; and/or
   containing said at least one thaumatin and said at least one sugar in a mass ratio of thaumatin:sugar of
   from 1:2,000 to 1:80,000,
   preferably from 1:4,000 to 1:65,000,
   more preferably from 1:6,000 to 1:55,000,
   even more preferably from 1:8,000 to 1:45,000, and
   even more preferably from 1:10,000 to 1:35,000.
47) The composition according to item 46, comprising said at least one thaumatin in a range of from 10 to 100 ppm per total weight of the composition and said at least one sugar in a range from 30% to 99.9%, preferably from 40 to 98%, more preferably from 50 to 90%, and even more preferably from 60 to 80% per total weight of the composition.
48) The composition according to any one of items 45 to 47, comprising a further component for said product for oral consumption, said component being one or more components selected from the group consisting of citric acid or a salt thereof, a vitamin, an inorganic salt, a trace element, caffeine, taurine, a gelling or thickening agent, a flavoring agent, and a preservative.
49) A use of a composition as defined in any one of items 45 to 48 as a sweetening composition or for preparing a product for oral consumption.
50) A method of reducing the content of sugar in a product for oral consumption, the sugar being selected from the group consisting of sucrose, glucose, and fructose, the method comprising replacing a part of said sugar with thaumatin in a range from 1 ppm to 13 ppm per total weight of the final product, preferably from 3 ppm to 7 ppm, wherein the thaumatin is selected from the group consisting of thaumatin I and thaumatin 11.
51) A method of reducing the dry taste of a HFCS-containing product for oral consumption, the method comprising adding a thaumatin to a pre-product of said product, said pre-product having a reduced content HFCS, wherein the thaumatin is selected from the group consisting of thaumatin I and thaumatin II.
52) The product, the composition, the use or the method according to any one of items 39 to 51, wherein said thaumatin is a protein comprising a polypeptide the amino acid sequence of which is that of SEQ ID NO: 5 or SEQ ID NO: 6, or an amino acid sequence having from 1 to 3 amino acid substitutions, additions, deletions, and/or insertions in the amino acid sequence of SEQ ID NO: 5 or 6, said thaumatin is preferably extracted from Nicotiana species.
53) Plant extract comprising thaumatin selected from thaumatin I and thaumatin II and a sugar selected from the group consisting of sucrose, glucose, and fructose, wherein the extract is not an extract of a Thaumatococcus daniellii plant.

The inventors of the present invention have conducted sensory evaluation studies and have found that the content of sugar in a product for oral consumption such as food or a drink can be reduced by up to 50%, even up to 60%, with minimal changes in taste properties when up to 60%, preferably up to 50% of the sugar is replaced by sweetness provided by thaumatin selected from the group consisting of thaumatin I and thaumatin II. Herein, the sugar to be reduced is selected from the group consisting of sucrose, glucose, and fructose. Further, the inventors have identified a method of reducing the sugar content in products for oral consumptions such as food or drinks with only minimal changes in the taste properties, including sweetness.

In addition, the invention provides a method of producing thaumatin I or thaumatin II with a purity of >95% by weight. The method comprises recombinant expression of thaumatin I or thaumatin II in plants, for example Nicotiana benthamiana, followed by extraction and purification. This method offers an environmentally friendly production of thaumatin compared to harvesting the katemfe (T. daniellii) plants in the wild. The method is scalable and more cost-effective compared to thaumatin produced by fermentation. Consequently, the method increases the availability of thaumatin for industrial applications and offers a high security of supply. These improvements in thaumatin production allow the use of thaumatin as a high potent sweetener and a taste modifier in the food industry.

### BRIEF DESCRIPTION OF THE DRAWINGS

FIG. 1 shows the schematic structure (A) and corresponding amino acid sequence (B) of Thaumatin-I preproprotein from Thaumatococcus daniellii (GenBank: BAF44567.1; SEQ ID NO: 1).
(A) The preproprotein consists of the cleavable N-terminal apoplast targeting presequence (TP) spanning amino acids 1-22, the mature protein fragment (Mature Protein) spanning amino acids 23-229, and cleavable C-terminal six-amino-acids tail (Tail) spanning amino acids 230-235. Numbers stand for amino acid positions, arrows show positions of cleavage.
(B) The arrow between amino acids 22 and 23 shows the position of cleavage between the N-terminal targeting presequence and the mature protein. The arrow between amino acids 229 and 230 indicates the position of cleavage between the mature protein and C-terminal six-amino-acid tail. Mature protein sequence is shown in bold.

FIG. 2 shows the schematic structure (A) and corresponding amino acid sequence (B) of Thaumatin-II preproprotein from Thaumatococcus daniellii(GenBank: AAA93095.1; SEQ ID NO: 3).
(A) The preproprotein consists of the cleavable N-terminal apoplast targeting presequence (TP) spanning amino acids 1-22, the mature protein fragment (Mature Protein) spanning amino acids 23-229, and the cleavable C-terminal six-amino-acids tail (Tail) spanning amino acids 230-235. Numbers stand for amino acid positions, arrows show positions of cleavage.
(B) The arrow between amino acids 22 and 23 shows the position of cleavage between N-terminal targeting presequence and the mature protein. The arrow between amino acids 229 and 230 indicates the position of cleavage between the mature protein and C-terminal six-amino-acids tail. Mature protein sequence is shown in bold.

FIG. 3 shows the alignment of amino acid sequences of Thaumatin-I and Thaumatin-II preproproteins (SEQ ID NO: 1 and SEQ ID NO: 3, respectively).

The cleavable N-terminal presequence and C-terminal tail are shown in boxes. Five mismatching amino acids are indicated by arrows. Th-II stands for Thaumatin-II preproprotein sequence; Th-I stands for Thaumatin-I preproprotein sequence. The alignment was performed using the Clustal Omega online tool accessed via URL https://www.ebi.ac.uk/Tools/msa/clustalo/.

FIG. 4 shows the alignment of amino acid sequences of mature Thaumatin-I and Thaumatin-II proteins (SEQ ID NO: 5 and SEQ ID NO: 6, respectively). Five mismatching amino acids are indicated by arrows. Th-I stands for mature Thaumatin-I protein sequence; Th-II stands for mature Thaumatin-II protein sequence. The alignment was performed using Clustal Omega online tool accessed via URL https://www.ebi.ac.uk/Tools/msa/clustalo/.

FIG. 5 shows schematically the T-DNA regions of pNMD40502 and pICH95397 constructs for the expression of Thaumatin-I and Thaumatin-II proteins, respectively. Expression vectors are based on Tobacco mosaic virus (TMV).

RB and LB stand for the right and left borders of T-DNA of binary vectors. Pact2: promoter of Arabidopsis actin2 gene; o: 5′ end from TVCV (turnip vein clearing virus); RdRp: RNA-dependent RNA polymerase open reading frame (ORF) from cr-TMV (crucifer-infecting tobamovirus); MP: movement protein ORF from cr-TMV; TP: apoplast targeting presequence from rice alpha-amylase 3A; Th-I(m): mature Thaumatin-I coding sequence; Th-II(m): mature Thaumatin-II coding sequence; N: 3′-non-translated region from cr-TMV; T: Agrobacterium nopaline synthase terminator, white segments interrupting grey segments in the RdRp and MP ORFs indicate introns inserted into these ORFs for increasing the likelihood of RNA replicon formation in the cytoplasm of plant cells, which is described in detail in WO2005049839.

FIG. 6 depicts double-inducible viral vectors for ethanol-induced expression of Thaumatin-I (pNMD40523) (A) and Thaumatin-II (pNMD38061) (B) used for stable transformation of Nicotiana benthamiana and Nicotiana tabacum plants. The T-DNA region of the plasmid contains four expression cassettes: 1) neomycin phosphotransferase II coding sequence cloned under the control of nopalin synthase promoter from Agrobacterium; 2) coding sequence of the ethanol-sensing transcriptional activator AlcR from Aspergillus nidulans (GeneBank: XM_677155.1) cloned under the control of potato ST-LS1 gene promoter (GenBank: X04753.1); 3) cr-TMV replicon (with deletion, indicated by the bracket, of a movement protein coding sequence fragment and insertion of Thaumatin ORF) cloned under the control of the ethanol-inducible alcohol dehydrogenase (alcA) promoter from Aspergillus nidulans fused with minimal 35S promoter sequence (Werner at al. 2011); and 4) cr-TMV movement protein coding sequence cloned under the control of alcA promoter.

RB and LB stand for the right and left borders of T-DNA of binary vectors. NosT stands for nopaline synthase terminator; NPTII: neomycin phosphotransferase II ORF for selection of transgenic plants; NosP: nopaline synthase promoter; Pstls: potato ST-LS1 gene promoter; Sntr: 5′ non-translated region; alcR: AlcR coding sequence from Aspergillus nidulans; 3ntr: 3′-non-translated region from cr-TMV; OcsT: terminator of octopine synthase gene from Agrobacterium; 35ST: cauliflower mosaic virus 35S terminator; Th-I(m): mature Thaumatin-I coding sequence; Th-II(m): mature Thaumatin-II coding sequence; TP: apoplast targeting presequence from rice alpha-amylase 3A; RdRp: RNA-dependent RNA polymerase open reading frame (ORF) from cr-TMV (crucifer-infecting tobamovirus); PalcA: ethanol-inducible alcA promoter from Aspergillus nidulans fused with minimal 35S promoter sequence; MP: movement protein ORF from cr-TMV. The position of MP deletion in TMV viral replicon is shown with brackets. Arrows indicate the direction of transcription.

FIG. 7 shows the diagram of a purification process for Thaumatin-I and Thaumatin-II produced in Nicotiana sp. plants.

FIG. 8 shows SDS-PAGE analysis of purification steps for Thaumatin-I protein produced in Nicotiana benthamiana. GJ: green juice (7.5 μl); CF: cleared filtrate (11.25 μl); CL: column load (11.25 μl); FT: flow through fraction (11.25 μl); E1: elution fraction 1 (11.25 μl); E2: elution fraction 2 (11.25 μl); E3: elution fraction 3 (3.75 μl); E4: elution fraction 4 (3.75 μl); E5: elution fraction 5 (11.25 μl); E6: elution fraction 6 (11.25 μl); E7: elution fraction 7 (11.25 μl); L: Protein Molecular Weight Ladder Mark 12™ (ThermoFischer Scientific, Waltham, Mass., USA).

FIG. 9 shows SDS-PAGE analysis of purification steps for Thaumatin-II protein. L: PageRuler™ Prestained Protein Ladder (ThermoFischer Scientific); GJ: green juice (10 μl); CF: cleared filtrate (10 μl); CL: column load (10 μl); FT: flow through fraction (20 μl); FT2: flow through fraction (after 17 liters column load) (20 μl); E: pooled elution fractions containing Thaumatin-II (5 μl).

FIG. 10 demonstrates results of capillary gel electrophoresis (CGE) analysis of the purity for Thaumatin-II preparation. Thaumatin-II is detected as a single band on the right side of the panel. Lane L: Protein 80 ladder (Agilent Technologies, Santa Clara, Calif., USA). Lanes 1 and 2: Bovine Serum Albumin (BSA) standard, reduced and non-reduced, respectively. Lanes 3 and 4 and 5 and 6 show replicates of Thaumatin-II (Th-II), reduced and non-reduced, respectively. Arrows show Thaumatin-II protein band.

FIG. 11 depicts electropherograms for capillary gel electrophoresis (CGE) analysis of Thaumatin-I (A) and Thaumatin-II (B) purity. X-axis labels indicate the size of protein molecular weight marker bands. Y-axis labels indicate fluorescence units (FU). Arrows show the peaks for Thaumatin-I (A) and Thaumatin-II (B). 97.85% and 98.2% protein purity was detected for Thaumatin-I and Thaumatin-II samples, respectively.

FIG. 12 shows the results of the ISD (In-Source Decay) and T3-sequencing analyses of Thaumatin-II batch #5 (A), batch #6 (B) and batch #7 (C). Amino acids that were confirmed are shown in bold.

FIG. 13 illustrates CGE analysis of purified Thaumatin-II (batch #17) stability upon the storage as lyophilized powder. The analysis was performed in two replicates: Replicate 1 (A) and Replicate 2 (B). The CGE electropherograms show the Thaumatin-II (Th-II) peak and several other protein peaks, identified as follows: LM: lower marker; SP: system peak; Th-II: Thaumatin-II; UM: upper marker. The identity and percent purity of Thaumatin-II was followed over time in storage at 4° C. and at room temperature (−22° C.); room temperature graphs are shown. Thaumatin-II purity (calculated as an average of two replicates) was 96.20% at the beginning of storage (0 month) and 94.85% after 11 months of storage.

FIG. 14 shows results of sensory studies for mixtures of the sucrose with Thaumatin-I; sweetness intensity and sweetness aftertaste were evaluated. Sweetness intensity was estimated after holding samples in the mouth for 5 seconds before expectorating. Sweet aftertaste was evaluated every 20 seconds for 2 minutes. Time was controlled by the panel leader. 10% sucrose was used as a control. The solutions with same sucrose contents were grouped as a same line pattern on the graphs. Different levels of Thaumatin-I are shown as different shapes of the markers on the graph. X-axis shows the time in seconds after expectoration of tested solutions; Y-axis shows sweetness intensity in arbitrary units. 6% sucrose with 3.5 ppm Thaumatin-I solution had a similar sweetness intensity as the control 10% sucrose solution, but different sweet aftertaste pattern.

FIG. 15 shows results of sensory studies for mixtures of sucrose with Thaumatin-II; sweetness intensity and sweetness aftertaste were evaluated. Sweetness intensity was evaluated after holding samples in the mouth for 5 seconds before expectorating. Sweet aftertaste was evaluated every 20 seconds for 2 minutes. 10% sucrose was used as a control. The solutions with same sucrose contents were grouped as a same line pattern on the graph. Different levels of Thaumatin-II are shown as different shapes of the markers on the graph. X-axis shows the time in seconds after expectoration of tested solutions; Y-axis shows sweetness intensity in arbitrary units. 5% sucrose with 5 ppm Thaumatin-II solution had sweetness intensity similar to the control 10% sucrose solution, but different pattern for the sweet aftertaste. If compared to the control, 5% sugar with 3.5 ppm Thaumatin-II solution had lower sweetness intensity; however it showed quite similar pattern for the sweet aftertaste.

FIG. 16 illustrates a comparison of sweetness between Thaumatin-I and Thaumatin-II. X-axis labels indicate tested samples. C: 10% sugar (control); 1: 5% sugar, 3.5 ppm Thaumatin-I; 2: 5% sugar, 3.5 ppm Thaumatin-II; 3: 5% sugar, 7 ppm Thaumatin-I; 4: 5% sugar, 7 ppm Thaumatin-II; 5: 6% sugar, 3.5 ppm Thaumatin-I; 6: 6% sugar, 3.5 ppm Thaumatin-II; 7: 6% sugar, 7 ppm Thaumatin-I: 8: 6% sugar, 7 ppm Thaumatin-II; 9: 7% sugar, 3.5 ppm Thaumatin-I; 10: 7% sugar, 3.5 ppm Thaumatin-II; 11: 7% sugar, 7 ppm Thaumatin-I; 12: 7% sugar, 7 ppm Thaumatin-II. Y-axis labels show sweetness intensity in arbitrary units; exact sweetness value is shown above each bar. Y-axis labels show sweetness intensity in arbitrary units; exact sweetness value is shown above each bar.

FIG. 17 shows results of sensory studies for mixtures of HFCS with Thaumatin-II; sweetness intensity and sweetness aftertaste were evaluated. Sweetness intensity was evaluated after holding samples in the mouth for 5 seconds before expectorating. Sweet aftertaste was evaluated every 20 seconds for 2 minutes. 10% sucrose and 10° Brix HFCS were used as a control. The solutions with same sugar contents were grouped as a same line pattern on the graph. Different levels of Thaumatin-II are shown as different shapes of the markers on the graph. X-axis shows the time in seconds after expectoration of tested solutions; Y-axis shows sweetness intensity in arbitrary units. 6° Brix HFCS with 2 ppm Thaumatin-II and 5° Brix HFCS with 3.5 ppm Thaumatin-II solutions had sweetness intensity and sweet aftertaste patterns closest to the control 10% sugar solution.

### DETAILED DESCRIPTION OF THE INVENTION

### Sugar of the Invention

The sugar of the invention is selected from sucrose, glucose, and fructose. These sugars are the main sweetening sugars in products for oral consumption that provide, at the same time, a high caloric content to products if these sugars are present in the products in high amount. There are other chemical compounds that can provide sweetness to products.

The presence of such other compounds that can provide sweetness to a product of the invention is not excluded. Herein, the term “glucose” refers to D-glucose. Otherwise, the glucose is not limited and comprises glucose in open-chain form and in cyclic form. The cyclic form may be present as α-D-glucose or as p-D-glucose. For determining the amount or content of glucose, the amounts or contents of all these forms of D-glucose in a product or composition are added. Compounds containing a glucose moiety as part of a larger molecule such as glucosides are not glucose in the sense of the present invention.

The term “fructose” refers to D-fructose. Otherwise, the fructose is not limited and comprises fructose in open-chain form and in cyclic form. In cyclic form, it may be present as fructopyranose or a fructofuranose. The cyclic form may be present as α- or R-anomeric form. For determining the amount or content of fructose, the amounts or contents of all these forms of D-fructose in a product or composition are added. Compounds containing a fructose moiety as part of a larger molecule are not fructose in the sense of the present invention.

The term “sucrose” refers to α-D-glucopyranosyl-(1-2)-β-D-fructofuranoside.

For use in the invention, the sugar may be employed as pure compound(s) of sucrose, glucose and/or fructose and used in such form in the product, composition, methods and uses of the invention. Alternatively, the sugar of the invention may be part of a sugar composition containing the sugar. An example of such sugar composition is a sugar composition or extract isolated or processed from sugar plants such as sugar cane or sugar beets. Another example of a sugar composition is a composition produced from plants containing high amounts of starch such as corn, other cereal plants, or potatoes. Such sugar compositions can be produced from plants high in starch content by a process comprising hydrolyzing the starch obtained from starch-containing plant parts to obtain glucose. A preferred sugar composition is High Fructose Corn Syrup (HFCS). HFCS can be obtained from corn starch by enzymatic hydrolysis of starch to glucose, followed by isomerizing glucose to fructose. Enzymatic hydrolysis of starch can be done using amylase enzymes. Isomerization of glucose to fructose can be done using the enzyme xylose isomerase. The obtained glucose-fructose mixture may be further processed to obtain a glucose-fructose mixture of desired fructose and glucose content, e.g. by separating fructose and adding the separated fructose to a product of the isomerization process. HFCS contains fructose, glucose, water, and some glucose oligosaccharides. HFCS is commercially available in various fructose concentrations, for example as HFCS-42, HFCS-55, HFCS-65, HFCS-70 or HFCS-90, wherein the number indicates the fructose content in mass-% of the dry composition (i.e. the solid content of the HFCS, after water removal). Thus, apart from fructose, the other components of HFCS are mostly glucose and water with some glucose oligosaccharides as mentioned above. A preferred HFCS is HFCS-42 containing 42% by weight fructose of the solid content of HFCS-42. Another preferred HFCS is HFCS-55 containing 55% by weight fructose of the solid content of HFCS-42.

The HFCS used in the invention may contain, per total weight of the HFCS, 22 to 25% by weight water and 78 to 75% by weight dissolved or dispersed solids, and said solids contain 15 to 92 weight % fructose, 8 to 85 weight % glucose, and 0 to 7 weight % glucose oligosaccharides per total weight of the solids, preferably said solids contain 40 to 65 weight % fructose, 30 to 55 weight % glucose, and 0 to 7 weight % glucose oligosaccharides per total weight of the solids.

### Thaumatin of the Invention

The thaumatin of the invention is selected from thaumatin I and thaumatin II, whereby thaumatin II is preferred. Both thaumatins are proteins, the amino acid sequence of which has a length of 207 amino acid residues of the mature form of these proteins as they occur in nature. The amino acid sequences of the natural forms of thaumatin I and thaumatin II are given in SEQ ID NO: 5 and SEQ ID NO: 6, respectively.

Herein, the term “thaumatin I” refers to a protein, the amino acid sequence of which is that of SEQ ID NO: 5 or that of an amino acid sequence having from 1 to 3, preferably 1 or 2, amino acid (residue) substitutions, additions, deletions, and/or insertions in the amino acid sequence of SEQ ID NO: 5. In a preferred embodiment, the thaumatin I is a protein, the amino acid sequence of which is that of SEQ ID NO: 5.

The term “thaumatin II” refers to a protein, the amino acid sequence of which is that of SEQ ID NO: 6 or that of an amino acid sequence having from 1 to 3, preferably 1 or 2, amino acid (residue) substitutions, additions, deletions, and/or insertions in the amino acid sequence of SEQ ID NO: 6. In a preferred embodiment, the thaumatin II is a protein, the amino acid sequence of which is that of SEQ ID NO: 6.

Where a thaumatin is defined herein by a number or numerical range of amino acid substitutions, additions, insertions and/or deletions, these amino acid substitutions, additions, Insertions and/or deletions may be combined, but the given number or numerical range refers to the sum of all amino acid (residue) substitutions, additions, insertions and deletions. Among amino acid substitutions, additions, insertions, and deletions, amino acid substitutions, additions, and deletions are preferred, substitutions and additions are more preferred, and additions are most preferred.

The term “insertion” relates to insertions within the amino acid sequence of a reference sequence, i.e. excluding additions at the C- or N-terminal end. The term “addition” means additions at the C- or N-terminal end of the amino acid sequence of a reference sequence. A deletion may be a deletion of a terminal or an internal amino acid residue of a reference sequence. The term “reference sequence” means the amino acid sequence of SEQ ID NO: 5 or SEQ ID NO: 6.

Thaumatin I and thaumatin II each has generally 8 intramolecular disulfide linkages. Therefore, any substitution or deletion with respect to a reference sequence is preferably not a substitution or deletion of a cysteine residue of a reference sequence, i.e. no cysteine residue is preferably substituted or deleted.

Preferred substitutions of thaumatin I of SEQ ID NO: 5 are substitutions selected from substitutions of K46, R63, R67, Q76, and D113.

A preferred substitution of K46 of SEQ ID NO: 5 is to N or R, preferably N.

A preferred substitution of R63 of SEQ ID NO: 5 is to K or S, preferably S.

A preferred substitution of R67 of SEQ ID NO: 5 is to K or H, preferably K.

A preferred substitution of Q76 of SEQ ID NO: 5 is to R or K, preferably R.

A preferred substitution of D113 of SEQ ID NO: 5 is to N, E or Q, preferably N.

Preferred substitutions of thaumatin II of SEQ ID NO: 6 are substitutions selected from substitutions of N46, S63, K67, R76, and N113.

A preferred substitution of N46 of SEQ ID NO: 6 is to K or R, preferably K.

A preferred substitution of S63 of SEQ ID NO: 6 is to K or R, preferably R.

A preferred substitution of K67 of SEQ ID NO: 6 is to R or H, preferably R.

A preferred substitution of R76 of SEQ ID NO: 6 is to Q or K, preferably Q.

A preferred substitution of N113 of SEQ ID NO: 6 is to D, E or Q, preferably D.

The generally known one-letter code for the 20 natural amino acid residues is used above.

Thaumatin I and II may be used singly or in combination. If thaumatin I and II are used in combination, the content given herein refers to the sum of thaumatin I and 11. Since the inventors have found that thaumatin II has better taste characteristics such as less lingering aftertaste, thaumatin II is preferred for use in the present invention. Therefore, the mass ratio of thaumatin II to thaumatin I in the product and composition of the invention is preferably at least 2:1, more preferably at least 4:1, even more preferably at least 9:1. If the mass ratio of thaumatin II to thaumatin I in the product or composition of the invention is 10:1 or higher, the product or composition is considered not to contain thaumatin I. Such low amounts of thaumatin I is not considered for determining the content of thaumatin in a product or composition of the invention.

Thaumatin I and II are highly water soluble (>20% w/v). The thaumatins may be dissolved/diluted in water or other suitable food-compatible vehicle or directly mixed into foods or beverages to achieve the desired effect of the invention.

The thaumatin for use in the invention may be stored as a dry lyophilized powder or in solution such as in water. The concentration of thaumatin in water or the stock solution may be measured using its absorbance at 280 nm due to tryptophan residues in thaumatin. An extinction coefficient of 29420 M⁻¹ cm⁻¹ is used both for thaumatin I and II based on 3 tryptophan residues per molecule. If the number of tryptophan residues per molecule is altered (e.g. due to a substitution of an amino acid), the absorbance coefficient is adjusted.

Thaumatins are natural sweet proteins present in the fruits of the katemfe plant (Thaumatococcus daniellii), a shrub growing in the undergrowth of West African forests. The fruits or, more specifically, the arils, contain the thaumatin as a mixture of different thaumatin proteins. Members of the thaumatin protein family were reported to be 2000-3000 times sweeter than sucrose on a weight by weight basis and are considered the sweetest natural substance known. Natural thaumatin from the katemfe plant has been used as sweetener and taste modifier in West Africa for centuries. At the molecular level, the electrical charge distribution on the thaumatin molecules may mediate their interaction with the taste receptors. The strength of the interaction between the thaumatin molecules and taste receptors may account for the intensity and the duration of sweetness perception of thaumatins.

In their natural plant of origin, thaumatins are secreted proteins and are translated as preproproteins (235 amino acids, 25.5 kDa) containing a cleavable N-terminal targeting presequence and a C-terminal six-amino-acid tail. Thaumatins I and II have similar properties, amino acid composition, sweetness, molecular weight (both are ˜22 kDa) and highly similar amino acid sequences, differing by only 5 amino acid residues. Each mature protein is a single polypeptide chain of 207 amino acids with 8 intramolecular disulfide linkages. X-ray crystallography of thaumatin I revealed the features of the protein's backbone. Circular dichroism studies showed few α-helices, but many β-pleated sheet strands and bends. It is believed that the constrained structure of thaumatins is necessary for inducing sweetness sensation. Heat denaturation or cleavage of the disulfide bridges may result in loss of sweetness.

Thaumatins interact with taste receptors in the tongue to impart the neurophysiological sensation of sweetness. Natural sweetness perception occurs when a sugar such as sucrose or other sweetener dissolves in saliva and binds to the heterodimeric T1R2-T1R3 receptors, which belong to the G-protein-coupled receptors (GPCRs) family. These receptors have multiple binding sites that are activated upon interaction with compounds that elicit sweet taste. Different ligands to the receptor exhibit different binding properties on the same receptors, leading to varying perceptions of sweetness for the different proteins.

Despite their sweetness, thaumatins have mostly been used as taste modifier rather than sweetener because the availability from the natural source is limited. Attempts to cultivate the katemfe plant (T. daniellii) in other areas than West Africa have failed and the extraction of natural thaumatins from the fruits is laborious. Consequently, thaumatin is expensive with a selling price as high as $7,000 to 10,000 per kg. Further, the low availability as a raw product and the low security of supply limit the use of thaumatin as a sweetener or taste modifier at a large scale in the food industry. In addition, natural thaumatin preparations are a mixture of different thaumatin proteins and possibly other compounds, that provide a sweet taste but also have undesirable taste attributes like slow onset of sweetness, a lingering aftertaste, and liquorice-like off notes.

To solve these problems, the invention provides thaumatin from plant sources other than the katemfe plant, which allows providing thaumatin without off notes and with controlled composition, high purity, and reproducible quality. This, in turn, allows the food industry to provide and produce the product for oral consumption in high amounts and, at the same time, reproducible taste even on large scale. Method of producing thaumatin from plants is further described below.

### Products for Oral Consumption

A product for oral consumption according to the present invention may be any product, preparation, or composition, that is suitable for consumption through the mouth by humans or other mammals, preferably by humans. The product comprises multiple components (e.g. chemical compounds). The product comprises at least two components or chemical compounds, namely a sugar and a thaumatin. Generally, the product comprises at least five, preferably at least ten components. Since many products are made from natural sources such as plants and/or animals or parts thereof, the product generally contains many components or chemical compounds derived from the plants and/or animals. One or more components of the product interact with sweetness receptors in the mouth and cause a sensation of sweet taste. Generally, components of the product will also interact with other taste receptors in the mouth and may cause other organoleptic sensations.

Products for oral consumption of the present invention contain thaumatin selected from the group consisting of thaumatin I and thaumatin II and sugar selected from the group consisting of sucrose, glucose, and fructose. The product/composition may further contain sweeteners other than said thaumatin or said sugar. The content of thaumatin in the product is suitably chosen for a particular product and depends on the desired taste properties of the product. The content of thaumatin generally is within the range of from 0.5 ppm to 20 ppm, preferably from 1 ppm to 13 ppm, and more preferably from 3 ppm to 7 ppm per total weight of the product. The total weight of the product refers to the total weight of the product in a state ready for oral consumption. The product comprises both (i) a thaumatin selected from the group consisting of thaumatin I and thaumatin II, preferably thaumatin II, and (ii) at least one sugar selected from the group consisting of sucrose, glucose, and fructose. Among the sugars sucrose, glucose, and fructose, the product may contain only one of these three sugars, a combination of two of these but not the third one, or all three of these sugars. In one embodiment, the product contains sucrose, but no glucose and no fructose. In another embodiment, the product contains glucose and fructose, but no sucrose. In a further embodiment, the product contains sucrose, glucose, and fructose.

The product for oral consumption comprises, with respect to the total weight of the product, from 2 to 12 weight %, preferably from 3 to 10 weight %, more preferably from 4 to 8 weight % of said sugar selected from the group consisting of sucrose, glucose, and fructose. In a preferred embodiment, said at least one sugar is selected from the group consisting of glucose and fructose, wherein said product may not contain sucrose. The feature that the product does not contain sucrose (or another sugar) means that the product contains less than 0.5 weight % sucrose (or of the other sugar).

The product may contain said thaumatin (preferably thaumatin II) and said sugar in a mass ratio of the thaumatin to the sugar from 1:2,000 to 1:80,000, i.e. from 2,000 ppm to 80,000 ppm of the sugar per 1 ppm of the thaumatin. Preferably, said thaumatin and said sugar are contained in the product in a mass ratio range of the thaumatin:the sugar of from
1:4,000 to 1:65,000,
more preferably from 1:6,000 to 1:55,000,
even more preferably from 1:8,000 to 1:45,000,
even more preferably from 1:10,000 to 1:35,000, and
most preferably from 1:15,000 to 1:30,000.

In a preferred embodiment, the thaumatin is thaumatin II and no thaumatin I is present, and the above ranges relate to thaumatin II.

These mass ratio ranges of the thaumatin to the sugar can be combined with the sugar content given above. In one embodiment, the thaumatin and the sugar are contained in the product in a mass ratio of the thaumatin to the sugar of from 1:8,000 to 1:45,000, and the product contains, with respect to the total weight of the product, from 3 to 10 weight %, preferably from 4 to 8 weight % of the sugar selected from the group consisting of sucrose, glucose, and fructose. In another embodiment, the thaumatin and the sugar are contained in the product in a mass ratio of the thaumatin to the sugar of from 1:10,000 to 1:35,000, and the product contains, with respect to the total weight of the product, from 3 to 10 weight %, preferably from 4 to 8 weight % of the sugar. In a further embodiment, the thaumatin and the sugar are contained in the product in a mass ratio of the thaumatin to the sugar of from 1:15,000 to 1:30,000, and the product contains, with respect to the total weight of the product, from 3 to 10 weight %, preferably from 4 to 8 weight % of the sugar. Where the sugar is selected from the group consisting of glucose and fructose (and the product contains no sucrose), these preferred embodiments may also relate to such product. Where the sugar is sucrose (and the product does not contain glucose and fructose), these preferred embodiments may also relate to such product.

If both thaumatin I and thaumatin II are present, the amounts (and ranges thereof) given above refer to the sum of thaumatin I and thaumatin II. In one embodiment and as stated above, the mass ratio of thaumatin II to thaumatin I in the product and composition of the invention is at least 2:1, more preferably at least 4:1, even more preferably at least 9:1. In one embodiment, thaumatin I but no thaumatin II is present. In another embodiment, thaumatin II but no thaumatin I is present. In a preferred embodiment, the product contains thaumatin II but no thaumatin I, since, as found by the inventors, thaumatin II has improved taste characteristics compared to thaumatin I and is therefore better suited for lowering the sugar content in a product, while maintaining taste characteristics. The term “no thaumatin I is present” means that less than 0.1 ppm of thaumatin I is present in said product or that the ratio of thaumatin II to thaumatin I is 10:1 or higher. The term “no thaumatin II is present” means that less than 0.1 ppm of thaumatin II is present in said product or the ratio of thaumatin II to thaumatin I is 1:10 or lower.

In one embodiment, the product contains High Fructose Corn Syrup (HFCS) as a sugar composition that contains glucose and fructose. Such sugar does not contain sucrose. If the product contains sucrose from other sources than from the HFCS, such sucrose content is included when determining the content of sugar in the product. A product for oral consumption may contain 5-7% HFCS solids per total weight of the product and thaumatin in a range from 1 ppm to 4.5 ppm per total weight of the product, wherein the HFCS solids are the solid content of High Fructose Corn Syrup and the thaumatin is selected from the group consisting of thaumatin I and thaumatin II (the preferred embodiments relating to thaumatin given above may be combined with this embodiment).

The product for oral consumption according to the present invention may be any product suitable for oral consumption, particularly sweet products. The product for oral consumption is generally ready for oral consumption. Examples are a beverage (e.g. soft drink), beverage powder, drink, soft drink, yoghurt notably sweetened yoghurt such as fruit yoghurt, jam, marmalade, a beverage concentrate such as syrup, dessert, cake, biscuit, cookie, chocolate, candy, sweets, sugar confectionary, chewing gum, custard, pudding, jelly, filling jelly, pastry, pie, drops, processed foods, cereals, baked goods, or a medicament.

Further examples are wine or beer or other fermented or distilled beverages, potato-based snacks, breakfast cereals, chewing gum, ice cream, cocoa and chocolate products, breath mints, sugar decorations or icings, coatings or fillings, fine bakery items, food supplements or table-top sweeteners. The products of the present invention do not only comprise processed foods or drinks manufactured in an industrial process but also hand-made foods or drinks.

The present invention also provides table-top sugar or sugar for baking or cooking that comprises thaumatin and serves as an ingredient for a product for oral consumption. Further examples are drugs for oral administration, notably liquid drugs such as cough syrup or oral antibiotic solutions or suspensions. In a preferred embodiment, a product for oral consumption according to the present invention is a soft drink, for example cola or other lemonade.

The product of the invention generally has comparable sweetness to a corresponding or conventional product sweetened with sugar selected from the group consisting of sucrose, glucose, and fructose but not containing thaumatin. The product of the invention also has a reduced calorie content in comparison to a conventional product sweetened with said sugar but not containing thaumatin. The product may have a comparable taste with respect to sweetness, aftertaste or further taste notes like bitterness, saltiness, sourness or umami when scored by a panel of taste experts in a taste test in comparison to the same product/composition that was sweetened with sugar selected from the group consisting of sucrose, glucose and fructose instead of thaumatin.

The product of the present invention may be produced by incorporating the thaumatin and/or the sugar into a product precursor, for example by mixing or blending, so as to obtain the product of the invention having the content of thaumatin and sugar as described above. The thaumatin may be blended or mixed into the product precursor at any time of the production process. If production of the product involves a heating step for pasteurization or sterilization, it may be preferred to add the thaumatin after the heating step to avoid denaturation of the thaumatin. The thaumatin may have been expressed as described in the examples and may be stored in a dry lyophilized form. For producing the product, a stock solution of known concentration of thaumatin may be produced in water or an aqueous solution. As stated above, the concentration of the thaumatin in the water or the stock solution may be measured using its absorbance at 280 nm. The thaumatin may be added to a product precursor from a thaumatin stock solution to give the desired thaumatin concentration of the product. The thaumatin stock solution may contain both thaumatin I and II in the ratio of thaumatin I and II desired for the product. Preferably, only thaumatin II is used; the stock solution will in this case contain thaumatin II but no thaumatin I. The thaumatin stock may be sterilized before addition to a product precursor, e.g. by filtration.

### Sweetness of the Product of the Invention

The sweetness of the product of the invention may be within a value of from 4 to 15, preferably of from 6 to 13, more preferably from 8 to 12, and most preferably from 9 to 11. The sweetness is determined by a panel of taste experts, wherein a value of 9 is defined as the sweetness of a solution of 10 g sucrose in 90 g water and a value of 0 is defined as the sweetness of a solution of 0 g sucrose in 100 g water, as described in the Examples.

In one embodiment, the content of thaumatin in parts per million (ppm) and sugar selected from sucrose, glucose, and fructose per total weight of the product follow the relationship (1):

Sweetness=1.62(+/−0.41)+[0.74(+/−0.19)×Sucrose]+[0.25(+/−0.063)×Thaumatin]+[0.11(+/−0.028)×Sucrose×Thaumatin] (1)

wherein “Sucrose” is the content of sucrose in weight percent (%) per total weight of the product or the combined content of sucrose, glucose and fructose that is as sweet as the content of sucrose, “Thaumatin” is the content of the thaumatin selected from thaumatin I and II in ppm, and “Sweetness” is as defined above. In a special embodiment, the sugar is sucrose and “Sucrose” is the content of sucrose in weight percent (%) per total weight of the product.

In another embodiment, the content of the at least one thaumatin selected from the group consisting of thaumatin I and thaumatin II and sucrose, both per total weight of the product, follow the relationship (II):

Sweetness=1.62(+/−0.41)+[0.74(+/−0.19)×Sucrose]+[0.25(+/−0.063)×Thaumatin]+[0.11(+/−0.028)×Sucrose×Thaumatin]; (II)

wherein “Sucrose” is the content of sucrose in weight percent (%) per total weight of the product, “Thaumatin” is the content of the thaumatin selected from thaumatin I and II in ppm, and “Sweetness” is as defined above.

In another embodiment, the product contains HFCS such as HFCS-55, wherein the content of thaumatin in ppm and the content of HFCS, both per total weight of the product, follow the relationship (III):

Sweetness=8.073(+/−2.02)+[0.206(+/−0.052)×HFCS-55]+[0.149(+/−0.038)×Thaumatin]+[0.016(+/−0.004)×HFCS-55×Thaumatin] (III)

wherein “HFCS-55” is the mass of HFCS-55 solids in weight percent (%) per total weight of the product, “Thaumatin” is the content of the thaumatin selected from thaumatin I and II in ppm, and “Sweetness” is as defined above.

The above relationships preferably also apply to the methods of the invention, such as the method of producing a product for oral consumption described below.

### Composition for Producing the Product for Oral Consumption

The product of the present invention may be produced by incorporating the thaumatin and the sugar from the composition of the invention to a product precursor, for example by mixing or blending, so as to obtain the product of the invention having the content of thaumatin and sugar as described above. The composition of the invention (also referred to herein as “sweetening composition”) is suitable for producing the product for oral consumption of the invention. The composition comprises at least one thaumatin selected from the group consisting of thaumatin I and thaumatin II and at least one sugar selected from the group consisting of sucrose, glucose, and fructose. In one embodiment, the sugar is sucrose, and the composition does not contain glucose nor fructose. In an alternative embodiment, the sugar is selected from the group consisting of glucose and fructose, and does not contain sucrose. In a preferred embodiment, the composition comprises thaumatin and High Fructose Corn Syrup (HFCS) comprising glucose and fructose. The invention is particularly suitable for reducing the content of HFCS, since HFCS has a dryness aftertaste that can be reduced by replacing part of it by thaumatin.

The sweetening composition generally contains a ratio of thaumatin I and II desired for the product, preferably it contains thaumatin II but no thaumatin I. Additionally, the composition may contain the relative amounts of sucrose, fructose and glucose as well as the ratio of thaumatin to sugar, such that the relative amounts of sucrose, fructose and glucose desired for the product, as well as the ratio of thaumatin to sugar, is obtained in the product by adding the composition to a precursor of the product (also referred to as “precursor product”). Thus, the sweetening composition allows a simple production of the product of the invention in that a sweet low calorie product can be produced in a simple way.

Since the sweetening composition is diluted in concentration of sugar and thaumatin upon blending with a precursor product, the composition has a higher content of thaumatin and sugar than those given above for the product of the invention.

The composition may contain said at least one thaumatin in a range of from 10 to 150 ppm, preferably of from 15 to 100 ppm, more preferably of from 20 to 80 ppm, and most preferably of from 30 to 70 ppm per total weight of the composition.

Alternatively or additionally, the composition may contain said at least one sugar in a range from 30 to 99.9%, preferably from 40 to 98%, more preferably from 50 to 90%, and even more preferably from 60 to 80% by weight per total weight of the composition.

In one embodiment, the composition comprises the at least one thaumatin in a range of from 10 to 100 ppm per total weight of the composition and said at least one sugar in a range from 30 to 99.9%, preferably from 40 to 98%, more preferably from 50 to 90%, and even more preferably from 60 to 80% by weight per total weight of the composition.

In another embodiment, the composition comprises the at least one thaumatin in a range from 30 to 70 ppm per total weight of the composition and said at least one sugar in a range from 30 to 99.9%, preferably from 40 to 98%, more preferably from 50 to 90%, and even more preferably from 60 to 80% by weight per total weight of the composition.

The composition of the invention may further comprise other components to be introduced in the product of the invention. Examples of such components are one or more components selected from the group consisting of citric acid or a salt thereof, a vitamin, an inorganic salt, a trace element, caffeine, taurine, a gelling or thickening agent, a flavoring agent, and a preservative. A vitamin may be one or more selected from ascorbic acid or a salt thereof, a vitamin B family vitamin, or tocopherol or a derivative thereof; said inorganic salt may be selected from a sodium salt, a magnesium salt, a potassium salt, and a calcium salt; said trace element may be a zinc compound, an iron compound, or a copper compound.

The sweetening composition may be solid or liquid. If it is solid, it may be a mixture of dry (e.g. lyophilized) thaumatin and the solid sugar selected from sucrose, glucose, and fructose. The dry sweetening composition may optionally contain further components as indicated above. Alternatively, the sweetening composition is liquid and contains, apart from its components thaumatin, sugar, and optional further components, a liquid dispersing agent or solvent. The dispersing agent or solvent is generally water.

The sweetening composition contains a high amount of sugar so that sterilization of it before addition to the precursor product may not be necessary. However, also the sweetening composition may be pasteurized or sterilized e.g. by filtration if it is a liquid.

The composition of the invention may be a plant extract comprising a thaumatin selected from thaumatin I and thaumatin II, preferably thaumatin II, to which a sugar selected from the group consisting of sucrose, glucose, and fructose may be added.

The composition of the invention may be used as a sweeting composition, such as for the product of the invention. The composition may also be used for sweeting other materials. The composition may be used for baking of cakes or cookies or for cooking. The composition may also be used as a table-top sweetener for sweetening food or drinks before consumption. Moreover, the composition of the invention may be used for reducing the caloric content of a product for oral consumption

### Methods of the Invention

The invention allows reducing the sugar content of a conventional product, notably a sweet product, by replacing part of the sugar selected from sucrose, glucose, and fructose by thaumatin I and/or II. The sugar content of a conventional product may be reduced by from 20 to 60%, preferably from 30 to 50%, most preferably from 35 to 45%. Within this range, the sweetness of the product can well be preserved according to the invention, and the taste characteristics of the product do not change or change very little. As a consequence, the caloric content of the product can be strongly reduced and the taste characteristics largely maintained. Below the above ranges, the reduction of the caloric content may be insufficient. Above the above ranges, the taste characteristics of the product may change too much and other product characteristics such as texture, mouthfeel, tonicity, and/or preservation may deteriorate. The sugar content of the product having reduced sugar content is as given above.

In a method of reducing the content of sugar in a product for oral consumption, the method may comprise replacing a part of said sugar with thaumatin in a range from 1 ppm to 13 ppm per total weight of the obtained product, preferably from 3 ppm to 7 ppm, wherein the thaumatin is selected from the group consisting of thaumatin I and thaumatin II, preferably it is thaumatin II and does not contain thaumatin I.

The invention provides a method of reducing the content of sugar in a product for oral consumption by up to 60%, preferably by the percentage ranges given above, wherein the sugar is at least one selected from the group consisting of sucrose, glucose, and fructose, the method comprising replacing a part of said sugar with thaumatin in a range from 1 ppm to 13 ppm per total weight of the obtained product, preferably from 3 ppm to 7 ppm, wherein the thaumatin is selected from the group consisting of thaumatin I and thaumatin II.

The invention further provides a method of using thaumatin as a sweetener in a product for oral consumption, the method comprising adding thaumatin to a pre-product of said product in a range from 1 ppm to 13 ppm per total weight of the product, wherein the thaumatin is selected from the group consisting of thaumatin I and thaumatin II. Preferably, the invention provides a method of using thaumatin as a sweetener in a product for oral consumption, the method comprising adding the composition of the invention (sweetening composition) to a precursor product to produce a product containing thaumatin in a range from 1 ppm to 13 ppm per total weight of the product, wherein the thaumatin is selected from the group consisting of thaumatin I and thaumatin II.

The invention also provides a method of reducing the sugar content in a product for oral consumption while preferably maintaining the sweetness of the product, the method comprising replacing a part of the sugar selected from the group consisting of sucrose, glucose and fructose with at least one thaumatin selected from the group consisting of thaumatin I and thaumatin II, wherein the content of thaumatin and the sugar content follow the relationship (1) given above.

In a preferred embodiment, the sugar is sucrose and the method of reducing the sugar content in a product for oral consumption, while preferably maintaining the sweetness of the product, comprises replacing a part of the sugar sucrose with at least one thaumatin selected from the group consisting of thaumatin I and thaumatin II, wherein the content of thaumatin and the sugar content follow the relationship (II) given above.

In another embodiment, the sugar is HFCS, preferably HFCS-55, and the method of reducing the sugar content in a product for oral consumption, while preferably maintaining the sweetness of the product, comprises replacing a part of the HFCS with at least one thaumatin selected from the group consisting of thaumatin I and thaumatin II, wherein the content of thaumatin and the sugar content follow the relationship (III) given above.

The invention also provides a method of producing a product for oral consumption, comprising mixing the sweetening composition of the invention with other components of the product or with a precursor product. The obtained product may have the sweetness as defined above with regard to relationships (I), (II) or (III). The invention also provides a method of using thaumatin as a sweetener in a product for oral consumption, the method comprising adding thaumatin to a precursor product in a range from 1 ppm to 13 ppm per total weight of the product, wherein the thaumatin is selected from the group consisting of thaumatin I and thaumatin II.

The invention further provides a method of reducing the dry taste of a HFCS-containing product for oral consumption, the method comprising adding a thaumatin to a pre-product of said product, said pre-product having a reduced content HFCS, wherein the thaumatin is selected from the group consisting of thaumatin I and thaumatin 11.

The invention also provides a method of reducing the dry taste of High Fructose Corn Syrup (HFCS) in a product for oral consumption, the method comprising replacing a part of the HFCS with thaumatin, wherein the content of the HFCS in the obtained product is reduced to 5-7% per total weight of the obtained product and replaced with thaumatin in a range from 2 ppm to 3.5 ppm per total weight of the obtained product, wherein the thaumatin is selected from the group consisting of thaumatin I and thaumatin II.

A method of reducing the dry taste of High Fructose Corn Syrup (HFCS) in a product for oral consumption, the method comprising replacing a part of the HFCS with thaumatin, wherein the content of the HFCS solids in the obtained product is reduced by 30-50% and replaced with thaumatin in a range from 2 ppm to 3.5 ppm per total weight of the obtained product, wherein the thaumatin is selected from the group consisting of thaumatin I and thaumatin II.

The invention further provides a use of thaumatin for reducing the dry taste of High Fructose Corn Syrup (HFCS) in a product for oral consumption by reducing the content HFCS solids in the product to 5-7% per total weight of the obtained product and adding thaumatin in a range from 1 ppm to 4.5 ppm per total weight of the obtained product, wherein the thaumatin is selected from the group consisting of thaumatin I and thaumatin II.

In the above methods of reducing the dry taste of HFCS, the HFCS is preferably HFCS-55.

### Production of Thaumatin

Thaumatins are available from commercial sources, including Naturex, Beneo Palatinit, Natex, KF Specialty Ingredients and several others. The product Talin® was commercialized beginning in the 1970s by Tate & Lyle (UK) with an alleged sweetness of 1,600-2,700 times that of a 7-10% solution of sucrose. A preparation was sold in Japan under the brand San Sweet T-100®. Commercial thaumatin may be analyzed for purity, e.g. by capillary electrophoresis or gel electrophoresis for purity. If the purity is not sufficient, e.g. because it contains less than 95% thaumatin (determined e.g. by Coomassie staining of electrophoresis bands and reading the intensity of thaumatin bands), it may be further purified by the methods described in the examples.

The invention provides methods of producing thaumatin in plants different from the natural origin of thaumatin. A thaumatin according to the invention may be produced by known methods of protein expression in a plant expression system. For producing the thaumatin, a nucleotide sequence encoding it may be expressed in a suitable plant host organism. Generally, the thaumatin is expressed from a nucleotide sequence encoding a thaumatin preproprotein comprising the apoplast targeting sequence, the mature thaumatin fragment, and the cleavable C-terminal tail.

Plant expression systems usable for expressing a thaumatin are described in the Examples. A possible way of achieving expression of a nucleotide sequence of interest encoding a preproprotein of a thaumatin in plants is the use of self-replicating (viral) replicons containing the nucleotide sequence encoding the preproprotein. The coding sequence of the preproprotein may be codon optimized for expression in plants or in the particular plant used as expression host. Plant viral expression systems have been described in many publications, such as in WO2012019660, WO2008028661, WO2006003018, WO2005071090, WO2005049839, WO2006012906, WO02101006, WO2007137788 or WO02068664 and many more publications are cited in these documents. Various methods for introducing a nucleic acid molecule, such as a DNA molecule, into a plant or plant part for transient expression are known. Agrobacteria may be used for transfecting plants with the nucleic acid molecule (vector) or nucleic acid construct e.g. by agroinfiltration or spraying with agrobacterial suspensions. For references, see WO 2012019660, WO 2014187571, or WO 2013149726.

In embodiments wherein strong expression of a thaumatin is desired, a nucleic acid construct containing a nucleotide sequence encoding the preproprotein may encode a viral vector that can replicate in plant cells to form replicons of the viral vector. In order to be replicating, the viral vector and the replicons may contain an origin of replication that can be recognized by a nucleic acid polymerase present in plant cells, such as by the viral polymerase expressed from the replicon. In case of RNA viral vectors (referred to as “RNA replicons”), the replicons may be formed by transcription under the control of a promoter active in plant cells, from the DNA construct after the latter has been introduced into plant cell nuclei. In case of DNA replicons, the replicons may be formed by recombination between two recombination sites flanking the sequence encoding the viral replicon in the DNA construct, e.g. as described in WO00/17365 and WO 99/22003. If the replicon is encoded by the DNA construct, RNA replicons are preferred. Use of DNA and RNA viral vectors (DNA or RNA replicons) has been extensively described in the literature over the years. Some examples are the following patent publications: WO2008028661, WO2007137788, WO 2006003018, WO2005071090, WO2005049839, WO02097080, WO02088369, WO02068664. Examples of DNA viral vectors are those based on geminiviruses. For the present invention, viral vectors or replicons based on plant RNA viruses, notably those based on plus-sense single-stranded RNA viruses may be preferably used. Accordingly, the viral replicon may be a plus-sense single-stranded RNA replicon. Examples of such viral vectors are those based on tobacco mosaic virus (TMV), crucifer-infecting tobamovirus (cr-TMV), and potexvirus X (PVX). “Based on” means that the viral vector uses the replication system such as the replicase and/or other proteins involved in replication of these viruses. Potexvirus-based viral vectors and expression systems are described in EP2061890 or WO2008/028661.

The thaumatin or its preproprotein may be expressed in a multi-cellular plant or a part thereof, notably a higher plant or parts thereof. Both monocot and dicot (crop) plants can be used. Common plants usable for expressing the protein of interest include Nicotiana benthamiana, Nicotiana tabacum, spinach, Brassica campestris, B. juncea, beets (Beta vulgaris), cress, arugula, mustard, strawberry, Chenopodium capitatum, lettuce, sunflower, cucumber, chinese cabbage, cabbage, carrot, green onion, onion, radish, lettuce, field peas, cauliflower, broccoli, burdock, turnip, tomato, eggplant, squash, watermelon, prince melon, and melon. Preferred plants are spinach, chard, beetroot, carrot, sugar beet, Nicotiana tabacum, and Nicotiana benthamiana. In one embodiment, plants are used that do not normally enter the human or animal food chain such as Nicotiana species such as N. tabacum and N. benthamiana. In the invention, the thaumatin is not expressed in Thaumatococcus daniellii.

Generally, the thaumatin as a protein of interest is targeted to the apoplast of the plants or plant parts. For this purpose, the preproprotein generally contains, as an N-terminal pre-sequence, a targeting peptide.

In the process of producing a thaumatin, a thaumatin is, in the first step, expressed in a plant or cells of a plant. In the next step, plant material containing expressed thaumatin from a plant having expressed the thaumatin is harvested. Plant material may e.g. be leaves, roots, tubers, or seeds, or a crushed, milled or comminuted product of leaves, roots, tubers, or seeds. In step (iii), the thaumatin is extracted from the plant material using an aqueous buffer. This may include that the plant material is homogenized and insoluble material may be removed by centrifugation or filtration. Soluble components including the thaumatin will be extracted into the aqueous buffer to produce a thaumatin solution in the aqueous buffer. The thaumatin may be purified and analyzed as described in detail in the examples. The thaumatin may be obtained as a solution in water and stored in solution, preferably in a frozen state. Preferably, the thaumatin is lyophilized to dry powder form, since it can be stably stored for a long time in such form.

The invention provides an extract comprising thaumatin selected from thaumatin I and thaumatin II and a sugar selected from the group consisting of sucrose, glucose, and fructose, wherein said plant is preferably not Thaumatococcus daniellii. The extract contains a thaumatin from the plant having expressed the thaumatin and, generally, other components derived from the plant. The extract may be a liquid containing the thaumatin in aqueous solution. The aqueous solution may comprise further components such as buffer.

### Quantification of Sugars and Thaumatin in Products and Compositions

For making the products and compositions of the invention, as well as for practicing the methods and uses of the invention, the sugars can be quantified by weighing and added to the product or composition in the desired amounts or mixtures of sugars. Thaumatin, notably if present in dry lyophilized form, may also be quantified by weighing. In aqueous solution, the thaumatin may be quantified by its uv absorbance at 280 nm as described above and in the Examples.

If present in a product of the invention, thaumatin may, for example, be determined by SDS-PAGE of a sample of the product, followed by Western blotting. For Western blotting, polyclonal antiserum may be used using thaumatin as an antigen, as is generally known in the art. For calibrating the Western blotting, pure thaumatin as produced according to the Examples may be used.

The analysis of sugars in a product of the invention, such as food, is known in food technology, see e.g. the book “Food Analysis” from S. Susanne Nielsen (editor), Fifth Edition 2017, Springer International Publishing, corrected publication 2019; DOI 10.1007/978-3-319-45776-5, notably Chapter 19 “Carbohydrate Analysis”, pages 333-360, from which the following disclosure in partly taken.

For many foods, except beverages, drying may be a first step in sample preparation until constant weight is reached. The dried material may then be ground to fine powder, followed by extraction of lipids and other lipid-soluble substances. The dried, lipid-free sample may then be extracted with hot 80% (v/v) ethanol in the presence of precipitated calcium carbonate to neutralize any acidity (AOAC Method 922.02, 925.05). Most carbohydrates, especially those of low molecular weight, are soluble in 80% (v/v) ethanol. Polymers, and almost all polysaccharides and proteins are insoluble in hot 80% ethanol, allowing rather specific extraction of any mono-(glucose, fructose), di-(sucrose, lactose, maltose), tri-(raffinose), tetra-(stachyose), or other oligosaccharides (e.g., maltodextrins) present. Contaminants in the 80% ethanol extract can be removed by ion-exchange techniques.

The content of sucrose, glucose and fructose in food samples after extraction and cleanup can be determined chromatographically, e.g. by High-performance liquid chromatography (HPLC). HPLC provides qualitative analysis through comparison to a standard and quantitative analysis through peak integration. HPLC analysis is rapid, can tolerate a wide range of sample concentrations and provides a high degree of precision and accuracy. HPLC can measure complex mixtures of mono- and oligosaccharides. The use of HPLC to determine food and other carbohydrates has been reviewed comprehensively, for example in Montero C M, Dodero M C R, Sánchez D A G, Barroso C G (2004): Analysis of low molecular weight carbohydrates in foods and beverages: A review; Chromatographia 59:15. Sample preparation for HPLC analysis is described in the literature, for example in Peris-Tordajada M (2012): HPLC determination of carbohydrates in foods (Chapter 7) In: Nollet L M, Toldra F (eds): Food analysis by HPLC, 3rd edn. CRC Press, Boca Raton. Separation of carbohydrates by HPLC can be carried out by anion-exchange columns (AE-HPLC). Carbohydrates are very weak acids and generally have a pKₐ value in the range of from 12-14. Therefore, solutions of high pH ionize some of the carbohydrate hydroxyl groups, which allows sugar separation on columns with anion-exchange resins. A pulsed electrochemical detector (ECD) relying on oxidation of carbohydrate hydroxyl and aldehydic groups is suited for use with anion-exchange chromatography. Thus, AE-HPLC coupled to an ECD allows examination of carbohydrates in many food components and products.

Another possibility to measure carbohydrates and sugars in food samples after extraction are enzymatic methods. These methods have a high specificity for the carbohydrate determined, do not require high purity of the sample analyzed, have very low detection limits, do not require expensive equipment and are easily automated. However, enzymatic methods rely on spectrophotometry for quantitation and require clear solutions for precise measurements. Therefore, cleanup of the extract before analysis, for example by a Carrez-treatment, is recommended. Enzymatic methods for the specific determination of sucrose, glucose and fructose have been developed as kits and are commercially available from several manufacturers. These kits contain the necessary enzymes and reagents for the analysis and provide detailed instructions that need to be followed for correct results. These factors need to be considered during the measurement to obtain reliable results.

Enzymatic assays are particularly suitable for quantifying monosaccharides. Disaccharides may be hydrolyzed to the underlying monosaccharide constituents. For example, glucose and fructose can be directly quantified with a kit for enzymatic determination thereof. Sucrose, on the other hand, may need to be hydrolyzed enzymatically to glucose and fructose within the process of sucrose determination. Sucrose may then be quantified as glucose that was released from the hydrolyzed sucrose. Using a kit designed to measure sucrose, all the steps necessary for the sucrose quantification are conveniently comprised in the manufacturer's protocol.

There are two widely used principles for enzymatic tests: the glucose oxidase/perioxidase/dye method (GOPOD method) or the NADPH-method. In the GOPOD method, glucose oxidase oxidizes glucose using molecular oxygen to D-glucono-1,5-lactone (glucono-delta-lactone) and hydrogen peroxide. After addition of peroxidase and a colorless leuco dye, the peroxidase uses the hydrogen peroxide to oxidize the leuco dye to a colored compound, which is then measured spectrophotometrically. The NADPH-method uses hexokinase to phosphorylate glucose to glucose 6-phosphate (G6P) using ATP. The reaction mixture generally also contains glucose 6-phosphate dehydrogenase (G6PDH) and NADP+. G6PDH catalyzes the oxidation of G6P to D-gluconate 6-phosphate and reduction of NADP+ to NADPH, so that the amount of NADPH formed is equivalent to the amount of D-glucose originally present. The amount of NADPH formed may be determined by measuring the absorbance at 340 nm of NADPH. With the addition of invertase, which hydrolyzes the sucrose to glucose and fructose, both the GOPOD-method and the NADPH-method can quantify the amount of sucrose in the sample. Both methods may measure the sucrose as glucose that was released from the sucrose hydrolysis.

### EXAMPLES

### Example 1: Thaumatin-I and Thaumatin-II Sequences

Thaumatin-I and Thaumatin-II proteins are most abundant forms in natural thaumatin mixture derived from Thaumatococcus daniellii, These two proteins were expressed using our plant-virus based expression system. In Thaumatococcus, both proteins are translated as preproproteins containing cleavable N-terminal apoplast targeting presequence and C-terminal six-amino-acid tail.

Thaumatin-I preproprotein (GenBank: BAF44567.1; SEQ ID NO: 1) is encoded by nucleotide sequence SEQ ID No: 2 (GenBank: AB265690.1) (FIG. 1). Thaumatin-II preproprotein (GenBank: AAA93095.1, SEQ ID NO: 3) is encoded by nucleotide sequence SEQ ID No: 4 (GenBank: J01209.1) (FIG. 2).

Both Thaumatin-I and Thaumatin-II preproteins consist of 235 amino acids. Both preproproteins consists of a cleavable N-terminal apoplast targeting presequence (amino acids 1-22), a mature protein fragment (amino acids 23-229), and cleavable C-terminal six-amino-acid tail (amino acids 230-235) (FIGS. 1 and 2).

Mature proteins for both Thaumatin-I (GenBank: AAL83964.1; SEQ ID NO: 6) and Thaumatin-II (GenBank: AAA93095.1; SEQ ID NO: 6) consist of 207 amino acids. SEQ ID NO: 7 (GenBank: AF355098.1) is a fragment Thaumatin-I preproprotein coding sequence which corresponds to mature protein. Similarly, SEQ ID NO: 8 (GenBank: J01209.1) is a fragment Thaumatin-II preproprotein coding sequence which encodes mature protein.

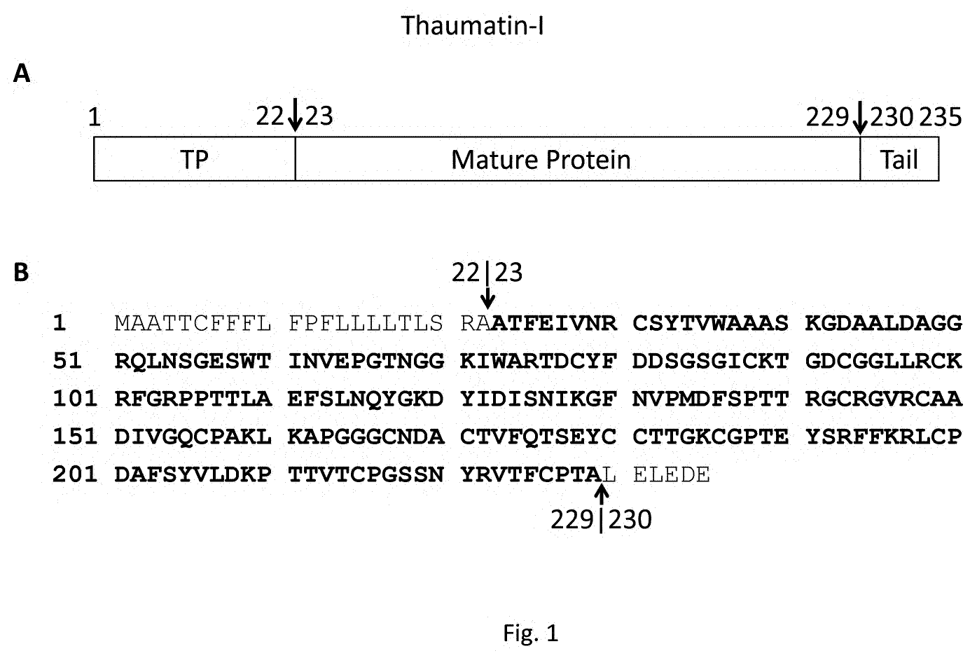

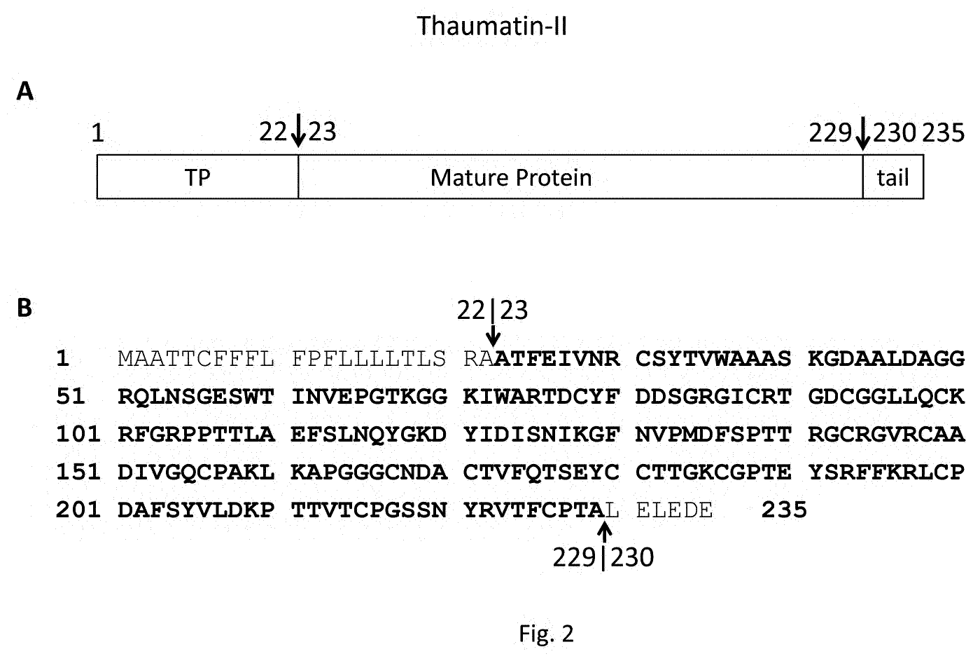

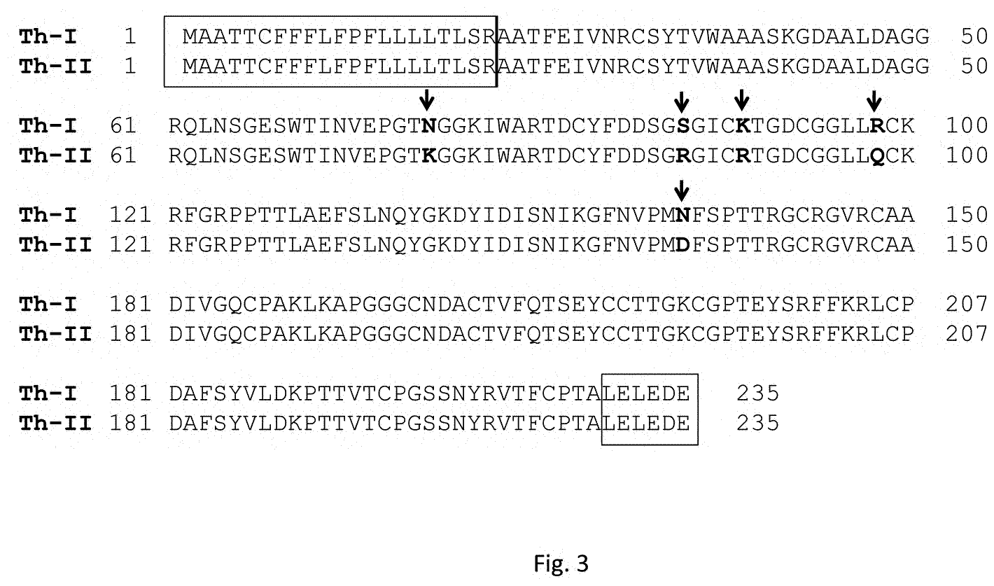

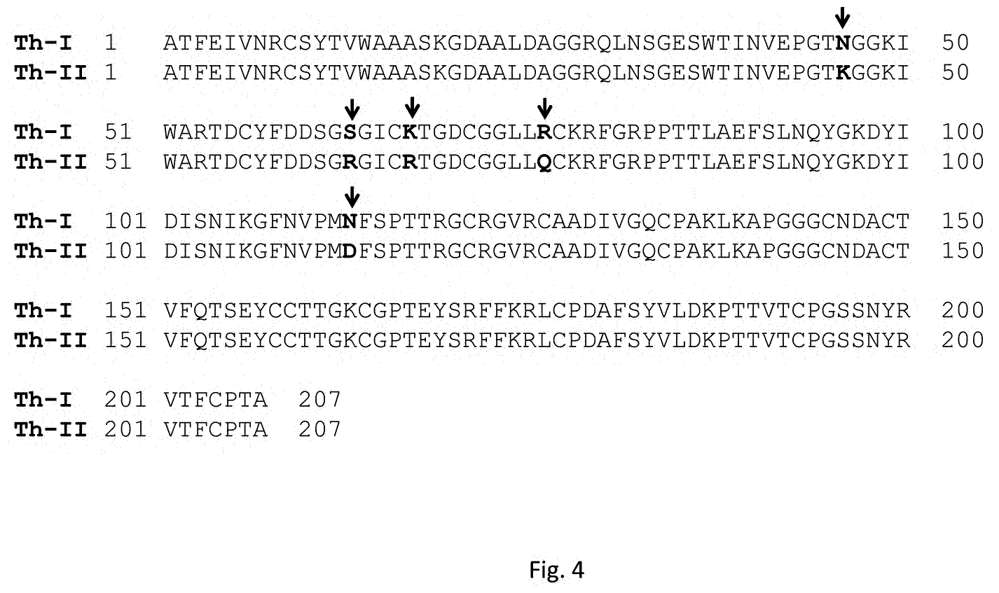

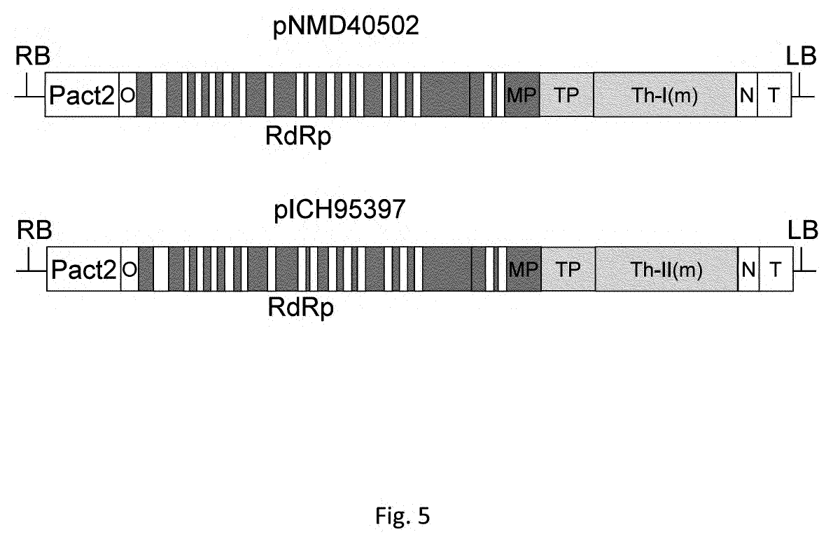

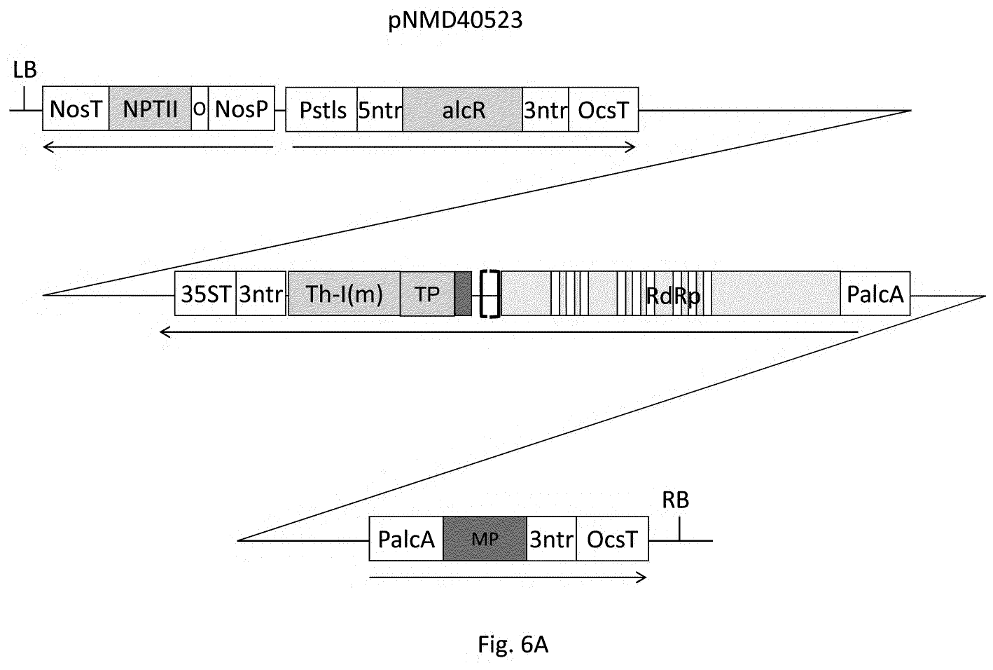

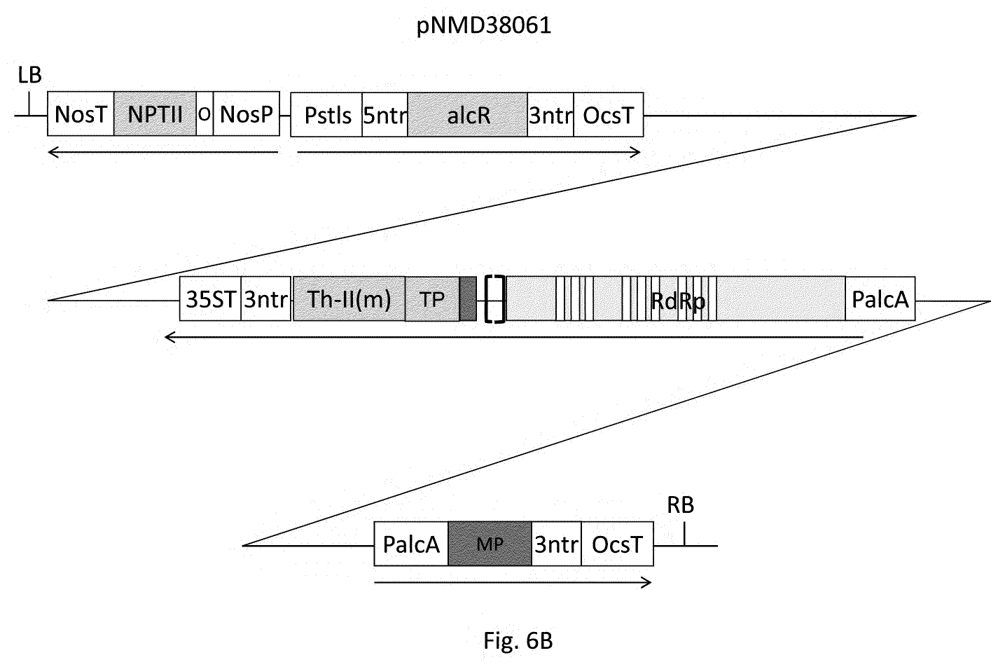

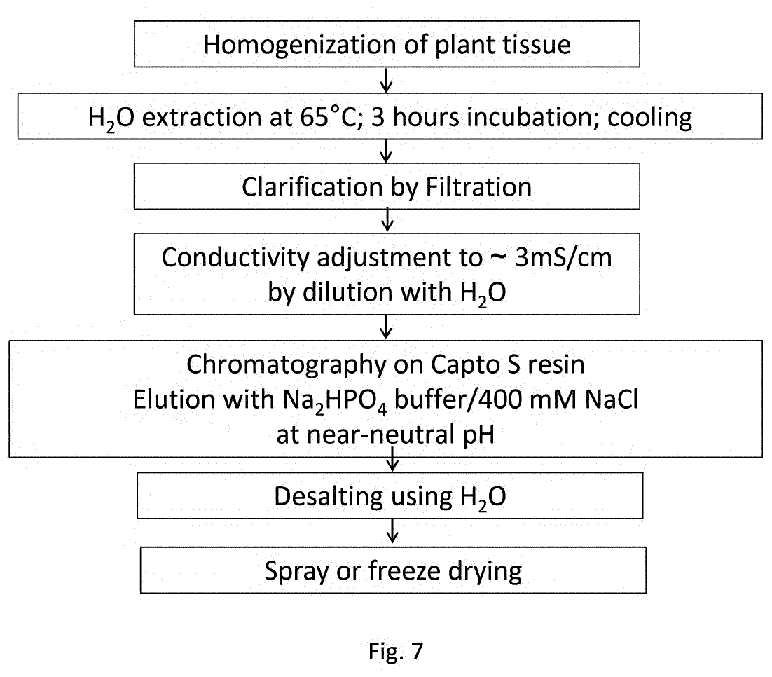

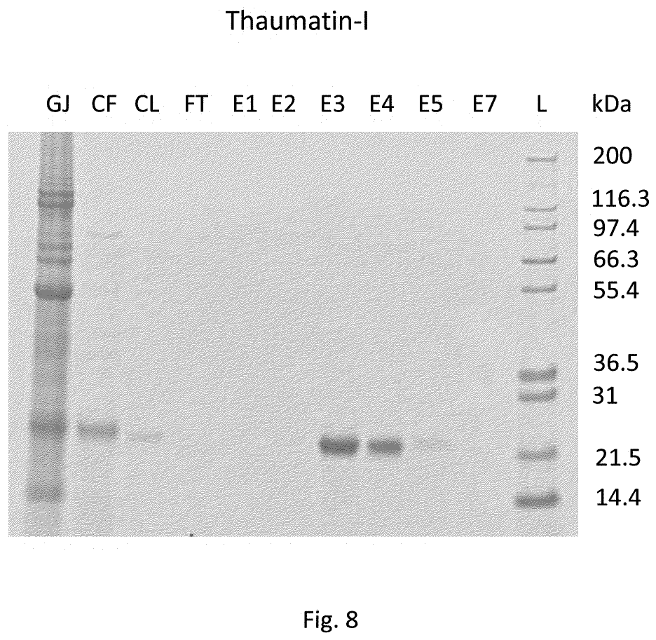

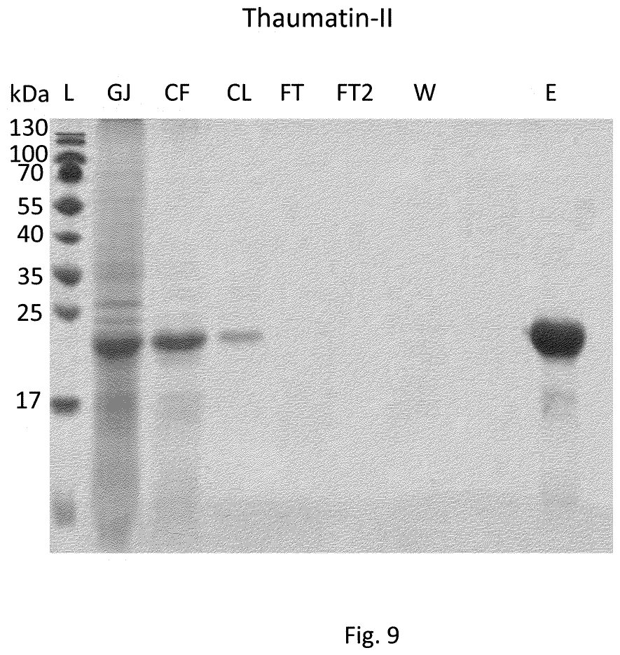

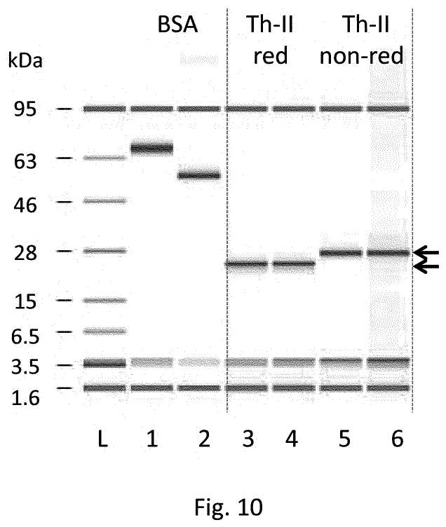

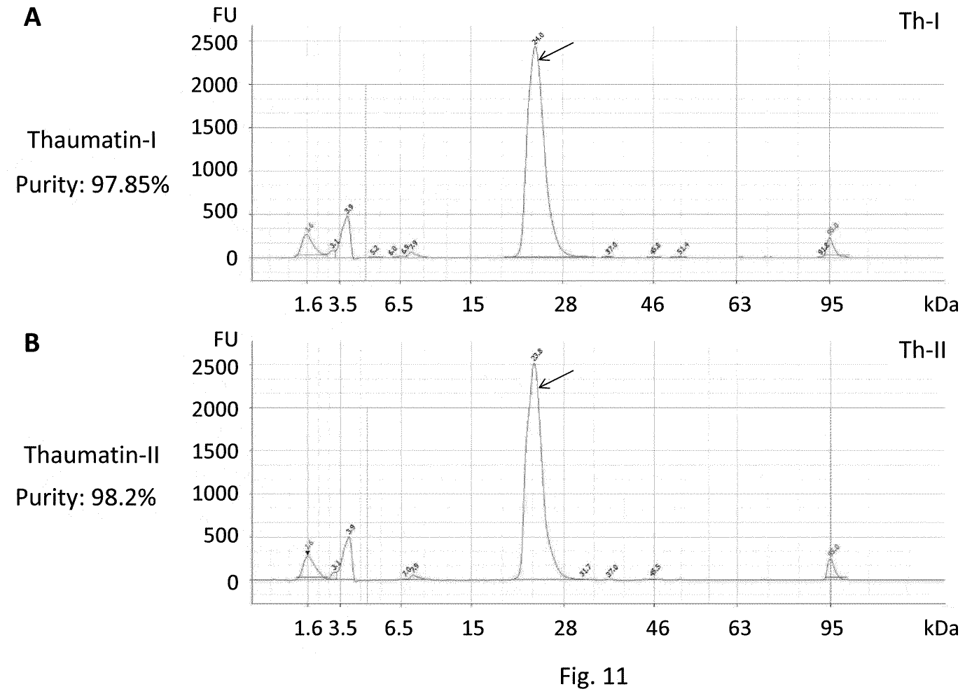

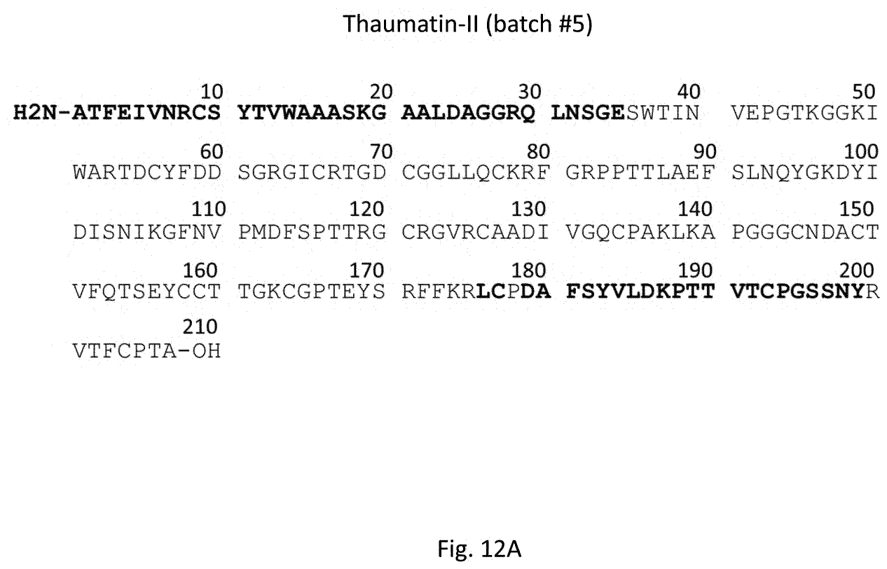

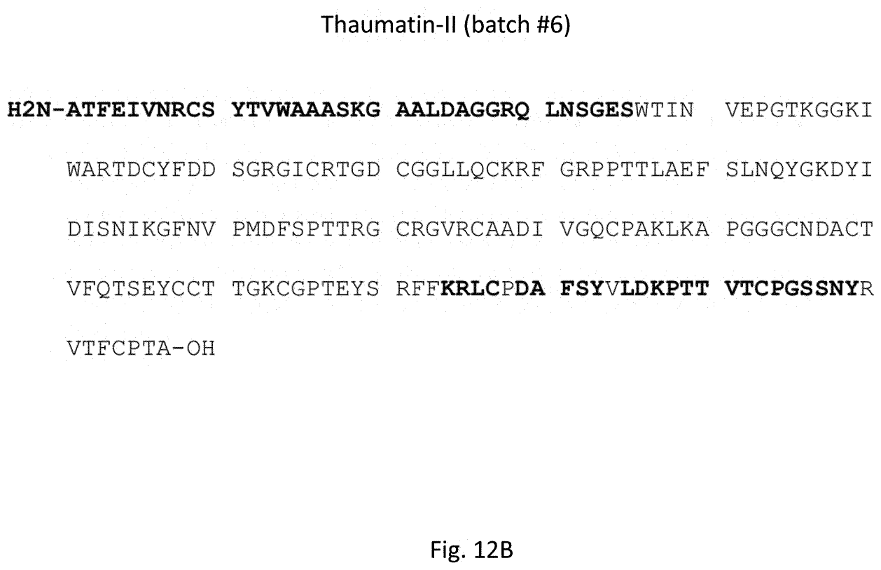

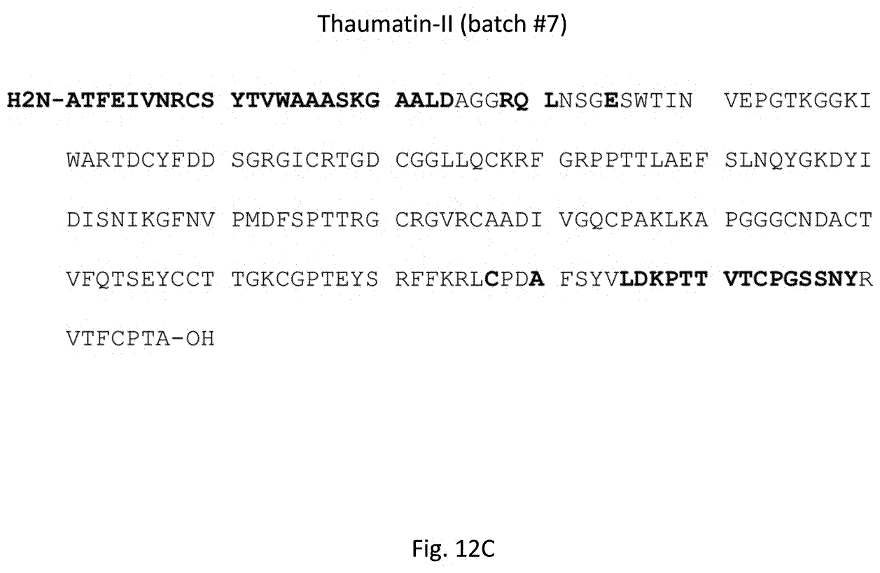

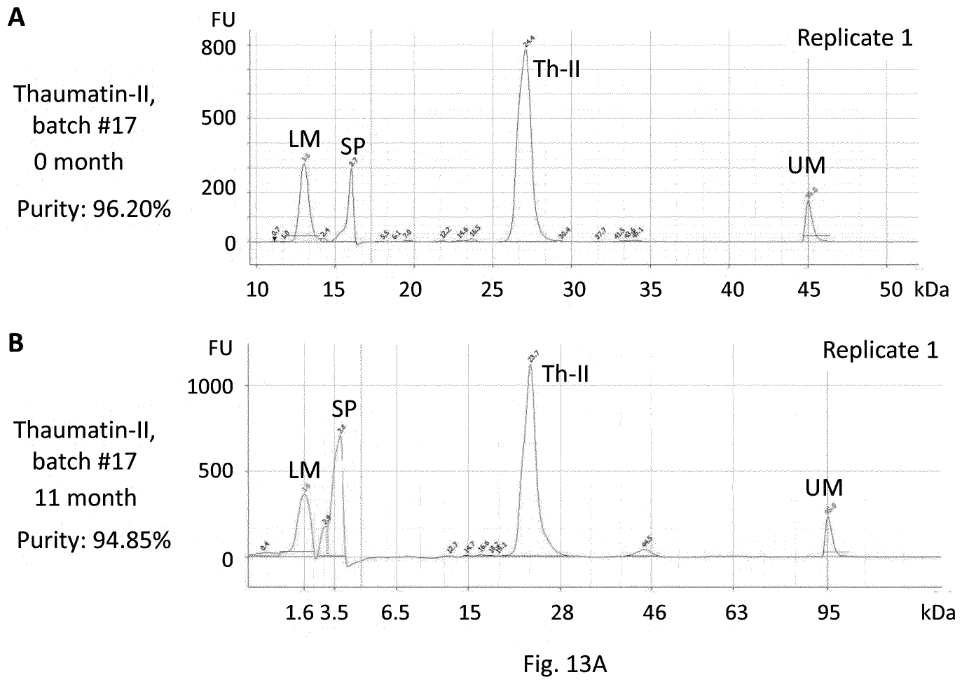

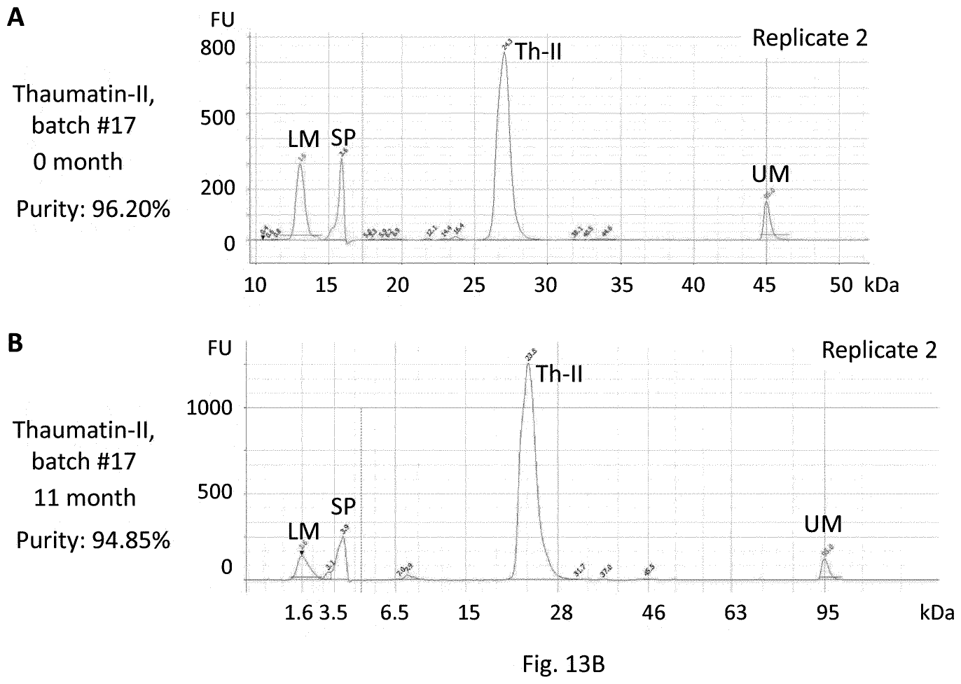

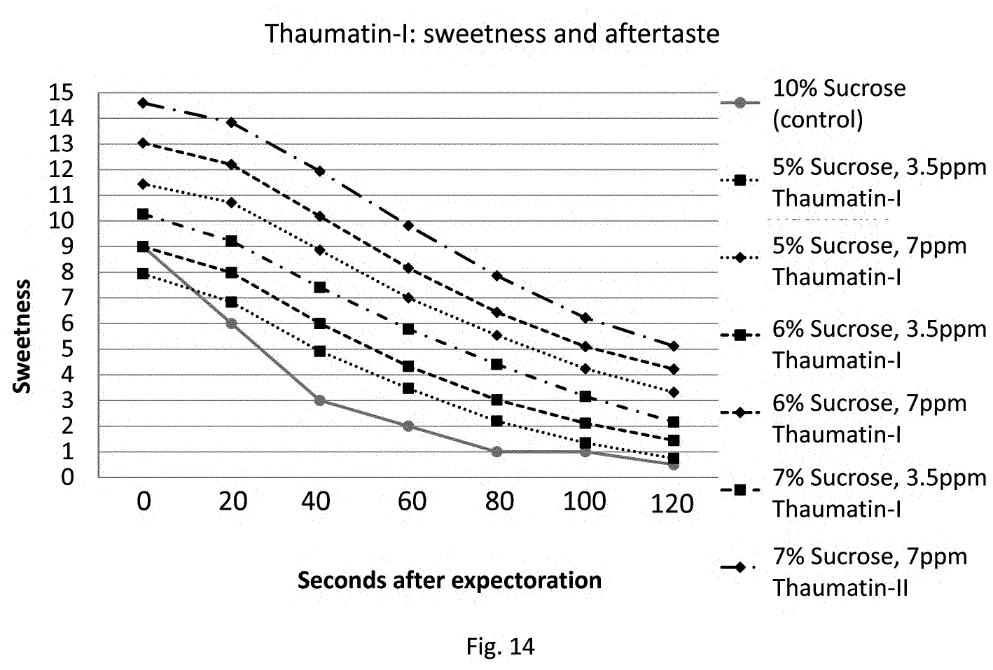

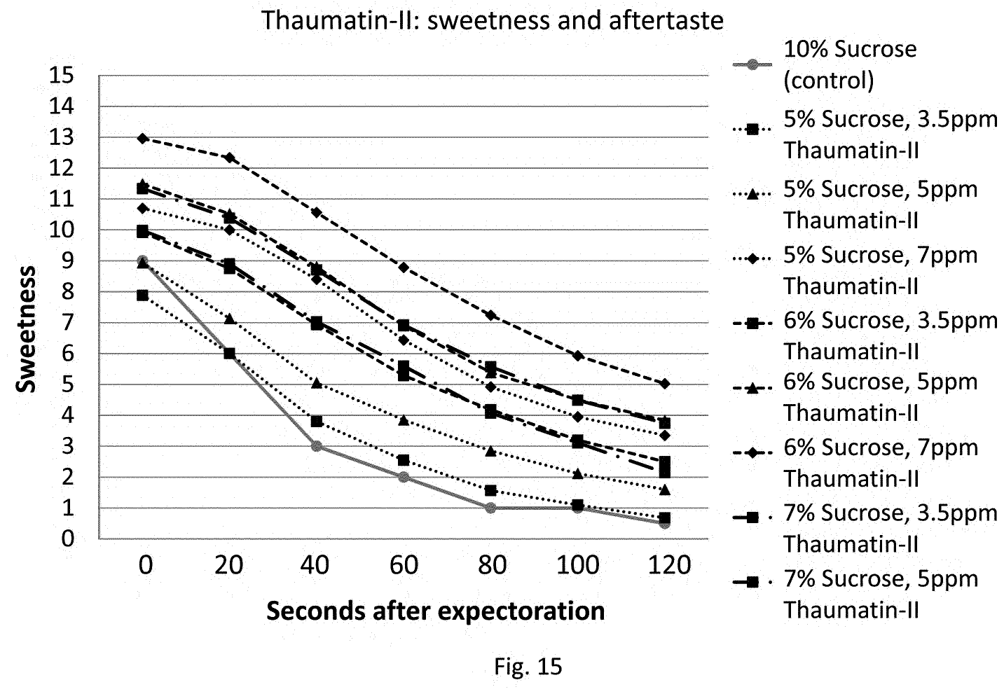

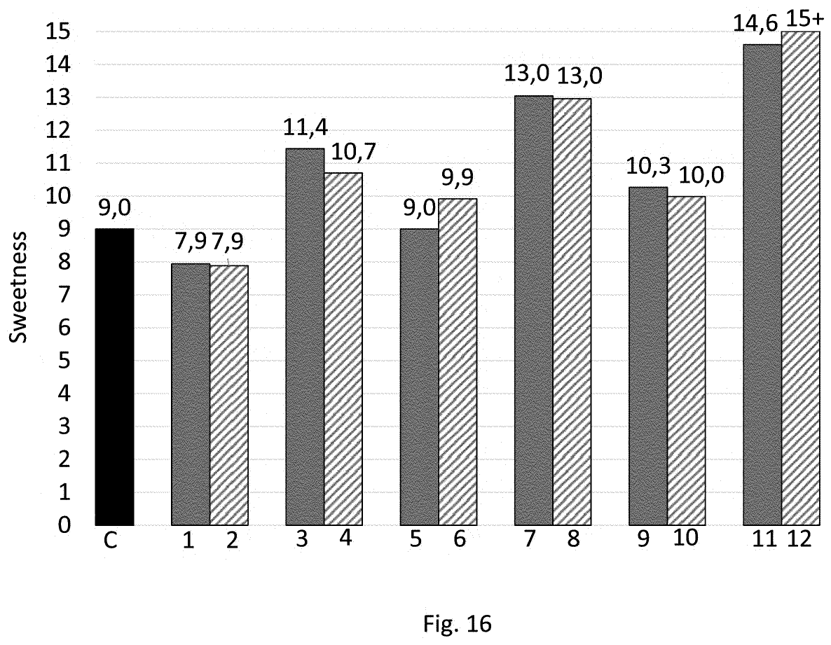

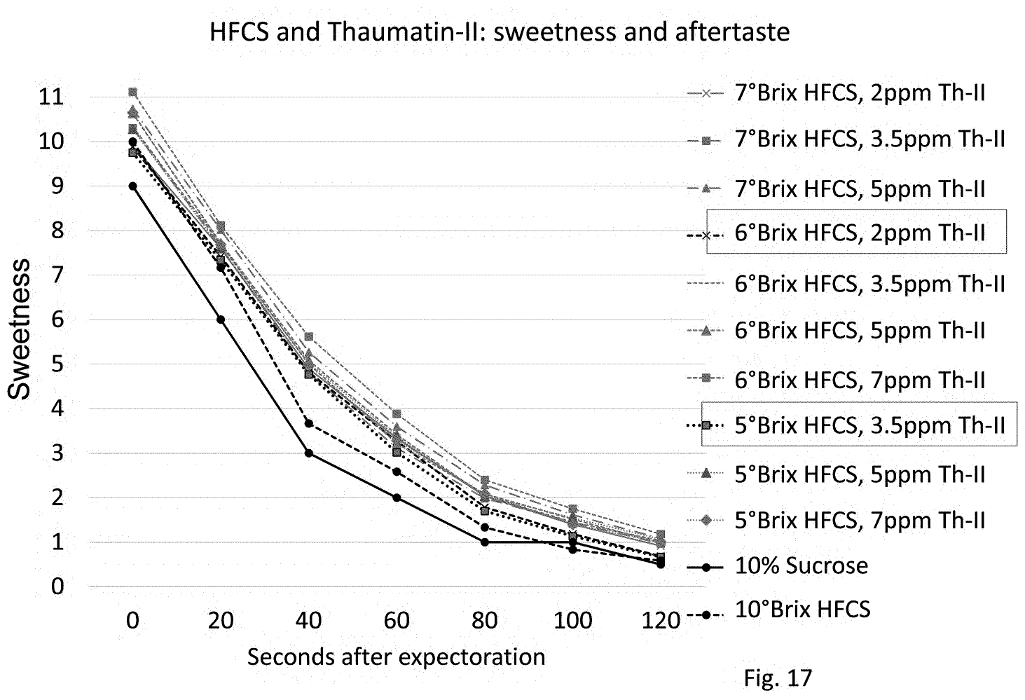

Thaumatin-I and Thaumatin-II preproproteins share 98.30% identity (Clustal Omega, standard settings), they differ in 5 amino acids only (FIG. 3). Both Thaumatin-I and Thaumatin-II preproproteins have identical cleavable N-terminal presequence and C-terminal tail, all 5 mismatching amino acids are located in mature proteins (FIGS. 3 and 4). Mature proteins share 98.07% identity (Clustal Omega, standard settings).

Calculated molecular masses of Thaumatin-I and Thaumatin-II with intact disulphide bonds are 22188.8 Da and 22271.9 Da, respectively.

### Example 2: Plasmid Constructs

Translational fusion of nucleotide sequence encoding N-terminal apoplast targeting presequence from Oryza sativa RAmy3A gene for alpha-amylase (GenBank: X56336.1) and coding sequence for mature Thaumatin-I (SEQ ID NO: 7) followed by stop-codon (SEQ ID NO: 9 for fusion sequence) were inserted into TMV-based assembled viral vector pNMD035 described in details in WO20121019660 patent. Resulting plasmid construct pNMD40502 is depicted in FIG. 6. SEQ ID NO: 11 is nucleotide sequence of T-DNA region of pNMD40502 vector. This construct was used for transient expression of Thaumatin-I protein using Agrobacterium-mediated delivery.

Similarly, translational fusion of the sequence encoding N-terminal apoplast targeting presequence from Oryza sativa RAmy3A gene for alpha-amylase and coding sequence for mature Thaumatin-II (SEQ ID NO: 8) followed by stop-codon (SEQ ID: 10 for fusion sequence) were inserted in pNMD035 plasmid resulting in pICH95397 construct (FIG. 6). SEQ ID NO: 12 is nucleotide sequence of T-DNA region of pICH95397 vector. This construct was used for transient expression of Thaumatin-II protein using Agrobacterium-mediated delivery.

Double-inducible viral vectors for ethanol-induced Thaumatin expression were created using the Golden Gate Modular Cloning approach (Engler et al. 2009; Weber et al. 2011; WO 2011/154147) as described in details in the European Patent Application published as EP3097783 A1. pNMD40523 construct (FIG. 6A) contained the insertion of translational fusion of the sequence encoding apoplast targeting presequence from rice alpha-amylase 3A and coding sequence for mature Thaumatin-I (SEQ ID NO: 9 for fusion sequence). SEQ ID NO: 13 is nucleotide sequence of T-DNA region of pNMD40523 construct.

pNMD38061 construct (FIG. 6B) contained the insertion of translational fusion of the sequence encoding apoplast targeting presequence from rice alpha-amylase 3A and coding sequence for mature Thaumatin-II (SEQ ID: 10 for fusion sequence). SEQ ID NO: 13 is nucleotide sequence of T-DNA region of pNMD38061 construct.

pNMD40523 and pNMD38061 constructs were used for stable transformation of Nicotiana benthamiana and Nicotiana tabacum plants.

### Example 3: Transient Expression of Thaumatin-I and Thaumatin-II in Nicotiana benthamiana with TMV-Based Viral Vectors

Nicotiana benthamiana plants were grown in the greenhouse (day and night temperatures of 19-23° C. and 17-20° C., respectively, with 12 h light and 35-70% humidity). Six-week old plants were used for inoculations with Agrobacteria.

The Agrobacterium tumefaciens inoculum carrying the selected thaumatin replicon was applied to greenhouse-grown and quality tested host plants through the stomata (pores) in the leaves. Inoculation of entire plants was accomplished by either vacuum-mediated infiltration after immersing the plant leaves in a suspension of the inoculum (Marillonnet et al. 2005, or via a procedure wherein the inoculum is sprayed onto plant leaves mixed with a surfactant (Hahn et al. 2015). Via either method, the agrobacteria are efficiently internalized into the plant and gain systemic distribution.

For vacuum infiltration, Agrobacterium tumefaciens ICF320 cells harboring the plasmid were inoculated to 300 ml of Luria-Bertani medium containing 50 mg/mi rifampicin and 50 mg/ml kanamycin (selection for the binary vector) and grown to saturation. Saturated Agrobacterium overnight cultures were adjusted to OD600=1.3 to 1.5 (approximately 1.2×10⁹ cfu/mL) with Agrobacterium inoculation solution (10 mM 2-[N-morpholino]ethanesulfonic acid (MES) pH 5.5, 10 mM MgSO₄). Bacterial culture was further diluted with same solution in order to get a 10⁻²-fold concentration relative to original culture. A beaker containing the infiltration solution was placed in a vacuum chamber (30-cm diameter), with the aerial parts of a plant dipped into the solution. A vacuum was applied for 2×15 sec using a Vacuum Pump ME 8 NT (Vacuubrand®, Wertheim, Germany) with pressure ranging from 0.15 to 0.2 bar. Infiltrated plants were returned to the greenhouse under standard conditions. Harvesting of aerial plant material was performed at 7 days post infiltration (dpi).

For spraying transfection, saturated Agrobacterium overnight cultures were adjusted to OD600=1.3 or 1.5 with Agrobacterium inoculation solution, further diluted with same solution supplemented with 0.1% (v/v) Silwet L-77 (Kurt Obermeier GmbH & Co. KG, Bad Berleburg, Germany) to get a 10⁻²-fold concentration, and inoculation was carried out using a hand sprayer (Carl Roth GmbH+CO.KG, Karlsruhe, Germany). Sprayed plants were returned to the greenhouse under standard conditions. Harvesting of aerial plant material was performed at 10-12 days post spraying (dpi).

### Example 4: Ethanol-Inducible Expression of Thaumatin-I and Thaumatin-II in Stable Transgenic Nicotiana benthamiana Plants

For ethanol-inducible Thaumatin-I and Thaumatin-II expression, we generated stable transgenic Nicotiana benthamiana and Nicotiana tabacum plants containing the genomic insertion of a double-inducible TMV-based viral vector (the approach is described in Werner et al. 2011).

Construct pNMD40523 for Thaumatin-I expression was transformed into Nicotiana benthamiana and Nicotiana tabacum ‘Samsun’ plants with Agrobacterium-mediated leaf disc transformation and selection on kanamycin-containing medium using a slightly modified standard protocol (Horsch et al. 1895; Werner et al. 2011). Construct pNMD38061 for Thaumatin-II expression was transformed into Nicotiana benthamiana and Nicotiana tabacum ‘Samsun’ and ‘Burley B5’ plants using the same approach. Regenerated plants were transferred to the greenhouse and tested for Thaumatin-I and Thaumatin-II expression upon ethanol induction.

### Example 6: Thaumatin-i and Thaumatin-II Purification

For purification of Thaumatin-I and Thaumatin-II, the same procedure with minor modifications was used. Flow diagram of Thaumatin purification process is depicted in FIG. 7.

Up to 3.5 kg of plant material were homogenized using the Fruit Shredder “Fruit Shark 1.6” (VARES Mnichovice a.s., Mnichovice, Czech Republic). The homogenate was further mixed with 1 Volume of Extraction buffer. In case of Thaumatin-I, Extraction buffer consisted of 20 mM Na₂HPO₄ pH 6. In case of Thaumatin-II, Extraction buffer of next composition was used: 20 mM Na₂HPO₄ pH 6.5. Diluted homogenate was passed through Tomato Press (9006N, Reber, Luzzara Italy) to remove solids. As a result of this procedure, the Green Juice (GJ) was collected and further processed.

Green Juice was further incubated for approximately 3 hours at 65° C. in drying oven (the temperature of the extract was measured using thermometer; until a temperature of around 48° C. was reached). After incubation, Green Juice was consequently filtrated through Miracloth and through the triple filter of 45 μm pore size using Filter Press Pulcino 10-20×10, Rover Pompe, Italy). Conductivity of resulting Cleared Filtrate (CF) was measured and adapted to ˜3 mS/cm using deionized water to achieve efficient binding of the protein.

Diluted CF was further filtrated through triple filter with 0.25 μm pore size using Filter Press (Pulcino 10-20×10, Rover Pompe, Italy). This step yielded in cleared extract which was used for loading to chromatography column (Column Load, CL).

Chromatography purification of Thaumatin was performed on the strong cation exchange (CIEX) resin CaptoS (GE Healthcare Life Sciences, Munich, Germany) using AKTA™ pure system (GE Healthcare Life Sciences, Munich, Germany). For purification, column volume 250 ml and the flow rate of 12-14 mi/min were used. Before sample loading, the column was equilibrated with 5 column volumes of 20 mM Na₂HPO₄, pH 6.5. After the sample loading, the column was washed with five column volumes of equilibration buffer. Thaumatin was recovered using the step elution with 5 column volumes of elution buffer containing 20 mM Na₂HPO₄, pH 7.3 and 400 mM NaCl.

The eluate (E) was subjected to buffer exchange against Millipore water using UF/DF with 5 kDa Minimate™ Tangential Flow Filtration Capsule using Minimate™ TFF System (Pall Life Science, Ann Arbour, USA) until 90% of buffer exchange is achieved. Protein concentration was measured by determination of the absorption at 280 nm. The appropriate amount of desalted Thaumatin was aliquoted into 25 ml glass vials frozen at −80° C. and until used for freeze drying.

Protein samples for purification steps were analyzed using SDS-PAGE as shown in FIGS. 8 and 9.

### Example 6: Thaumatin-I and Thaumatin-II Quality Control: Protein Purity

The purity of isolated Thaumatin-1 and Thaumatin-III proteins was analysed by capillary gel electrophoresis (CGE).

Capillary gel electrophoresis (CGE)-on-a-chip analysis were performed on an Agilent 2100 bioanalyzer (Agilent Technologies Deutschland GmbH; Waldbronn, Germany) in combination with an Agilent Protein 80 Kit (sizing range: 5-80 kDa) and 2100 Expert Software (Kuschel et al. 2002). All reagents and chips were prepared according to the manufacturer's instructions.

Lyophilized, buffer containing Thaumatin-I and Thaumatin-II samples were reconstituted with water to a concentration of 1 mg protein per ml. 4 μl of each Thaumatin sample and 2 μl of reducing sample buffer were mixed and incubated at 95° C. for 5 min. After adding 84 μl water to each Thaumatin-buffer mix, 6 μl of each sample were loaded onto a chip together with two BSA standard protein samples (reduced and non-reduced) and a protein 80 ladder. The chip run results were displayed as a gel-like image, electropherograms and in tabular form. Peak baseline adjusting and peak integration of electropherograms were done automatically and, if necessary, manual adjusting of peak baselines was done on a case-by-case basis.

FIG. 10 shows gel image for GCE analysis of Thaumatin-II purity. Analysis was carried out in duplicates using BSA as a standard and a Protein 80 ladder. Proteins were separated under non-reducing and reducing conditions. FIG. 11 shows corresponding electropherograms for GCE analysis of Thaumatin-I (A) and Thaumatin-II (B) purity. Purity of proteins is given as percentage of total soluble proteins obtained upon resuspension of lyophilized protein samples. It was found to be in the range of 97%-98% for both proteins.

The concentration of purified Thaumatin-I or Thaumatin-II in water solution was determined based on the absorbance at 280 nm (A280) using the Lambert-Beer law. A280 was measured using BioTek™ Synergy™ HTX Multi-Mode Microplate Reader and Take3™ Multi-Volume Plate (BioTek Germany, Bad Friedrichshall, Germany). Extinction coefficient and absorption of 0.1% w/v (=1 g/l) solution were calculated using ProtParam tool (ExPASy Bioinformatics Resource Portal) accessed via URL https://web.expasy.org/cgi-bin/protparam/protparam. For both, Thaumatin-I and Thaumatin-III extinction coefficients were computed to be the same: 29420 M-1 cm-1 (non-reduced form). Absorption values of a 0.1% w/v solution in water slightly differed: 1.325 for Thaumatin-I and 1.320 for Thaumatin-III (both in non-reduced form).

### Example 7: Thaumatin-II Quality Control: Protein Integrity

To ensure Thaumatin-II integrity upon purification, reconstituted lyophilized proteins were analysed by MALDI-TOF/TOF mass spectrometry. Batches #5, #6 and #7 were used for analysis. For each batch, the molecular mass of Thaumatin-II was determined, and the N- as well as the C-terminal sequences were verified.

Sequence verification of the protein termini has required the use of a specialized mass spectrometry technique termed as in-source decay (ISD). This technique makes use of N-terminal (a- and c-type) and C-terminal (y- and z-type) fragment ions, which are generated due to highly elevated laser energy during ionization. These fragment ions can be used to derive the terminal amino acid sequences of proteins. ISD is an untargeted technique; hence it is not possible to influence the kind of generated fragments (C- and N-terminal, only N- or C-terminal) as well as the efficiency by which they are produced. If two different compounds are present within a sample, then fragment ions of both are usually observed. ISD spectra do not cover the first amino acids of the N- and C-terminus. Hence, they do not allow the identification/confirmation of the respective amino acids as well as the exact localization of possible modifications. To solve this issue the use of a technique termed as T3-sequencing is necessary. The T3 approach is based on the analysis of selected ISD fragments by LIFT. Since ISD fragment ions are generated within the ion source, they can further fragment inside the mass analyser. The LIFT unit, which is located within the mass analyser, makes use of this behaviour. LIFT specifically selects an ISD fragment ion and acquires a fragment spectrum of it, which usually allows the identification of the first amino acids and their modifications.

Native Thaumatin-II contains eight disulphide bonds. In order to investigate the presence of these disulphide bonds in the three batches, the respective samples were divided into two parts: one was directly applied onto the MALDI target (non-reduced sample) and the other one was treated with 10 mM DTT for 30 min at 50° C. (reduced sample). Both kinds of samples were co-crystallized on a MALDI ground steel target with the MALDI matrices S-DHB (mixture of 2.5-dihydroxybenzoic acid and 2-hydroxy-5-methoxybenzoic acid) and DHAP (2,5-dihydroxyacetophenone).

Mass spectra were acquired on a MALDI-TOF/TOF mass spectrometer (Autoflex Speed, Bruker Daltonics, Bremen, Germany) with positive polarity in linear (molecular mass determination) as well as in reflector mode (ISD analysis). Irradiation of the analyte-containing matrix was achieved by using a Nd:YAG laser (Smart beam-II, Bruker Daltonics, Bremen, Germany) set to a pulse rate of 1 kHz, a pulse energy of 500 μJ and an emission wavelength of 355 nm. Spectra were recorded using flexControl (Version 3.4, Bruker Daltonics, Bremen, Germany) by accumulation of at least 10000 shots (per sample spot). Laser energy was set slightly above the threshold for MS experiments and set to highly elevated values for ISD analyses. Spectra processing was carried out with flexAnalysis (Version 3.4, Bruker Daltonics, Bremen, Germany) by applying baseline subtraction with TopHat algorithm, smoothing with Savitzky-Golay algorithm and peak detection with SNAP algorithm. The MALDI-TOF/TOF mass spectrometer was calibrated using the mass signals of a set of standard peptides and proteins with known masses (Peptide Calibration Standard II, Protein Calibration Standard I and II, Bruker Daltonics, Bremen, Germany). Spectra employed for calibration were acquired with the same laser energy as used for sample analysis.

All three batches of Thaumatin-II were analysed by MALDI-TOF(/TOF) mass spectrometry in order to determine the molecular mass, to assess the integrity of the protein termini and to reveal information about the presence of disulphide bonds.

For each sample, the molecular mass was determined for Thaumatin-II in its non-reduced and its reduced state. The obtained mass values showed good correlation with the theoretical mass with deviations of usually less than 5 Da.

Comparison of experimentally determined masses of non-reduced and reduced Thaumatin-II revealed mass differences between 8.0 Da and 18.9 Da. These differences indicate the presence of disulphide bridges in non-reduced Thaumatin-II.

The results of ISD analysis and T3-sequencing confirmed that both protein termini were intact and no sequence variations or modifications were present (Table 1).

TABLE 1
Results of ISD analysis and T3-sequencing for Thaumatin-II samples.

| | Intact mass and mass deviation | | | | | |
|---|---|---|---|---|---|---|
| | Non-reduced | | Reduced | | | |
| | Mass avg | Dev. | Mass avg | Dev. | | |
| Batch | (Da) | (Da)* | (Da) | (Da)** | N-terminus | C-terminus |
| #5 | 22271.7 | −0.2 | 22279.7 | −8.4 | H₂N-ATFEI[. . .] | [. . .]FCPTA-OH |
| #6 | 22274.9 | 3.0 | 22291.0 | 3.0 | | |
| #7 | 22271.8 | −0.1 | 22290.7 | 2.7 | | |

*Deviation (Dev.) is a difference between average mass determined experimentally (Mass avg) and theoretical average mass (22271.9 Da for non-reduced protein).
**Deviation (Dev.) is a difference between average mass determined experimentally (Mass avg) and theoretical average mass (22288.0 Da for reduced protein).

FIG. 12 provides an overview about the confirmed amino acid residues. ISD and T3-sequencing data taken together confirm the integrity of Thaumatin-II protein in all three tested batches, correct cleavage of N-terminal apoplast targeting preseqence, integrity of both N- and C-termini and the presence of 8 disulphide bonds.

### Example 8: Stability of Purified Thaumatin-I and Thaumatin-II

The stability of purified, N. benthamiana-produced Thaumatin-I and Thaumatin-II protein powders was assessed during storage at 4° C. and at room temperature (˜22° C.). Stability was determined by CGE (capillary gel electrophoresis) using an Agilent 2100 Bioanalyzer and Agilent Protein 80 reagent kit (Agilent Technologies). For analysis, one milligram (1 mg) of stored purified Thaumatin protein powder produced in non-sequential batches sampled at various timepoints was dissolved in 1 ml water. Typical electropherograms of samples from a developmental batches of Thaumatin II stored at room temperature are shown in FIG. 13.

The percent purity of Thaumatin-I and Thaumatin-II was determined from analyses (average of duplicate replicate experiments) of non-sequentially produced batches sampled at various times of storage at two temperatures and referred to the stability of the proteins (reduced purity due to degradation). A compilation of results for Thaumatin-II is shown in Table 2.

TABLE 2
Stability of Thaumatin-II during storage.

| | Thaumatin-II Purity During Storage | | | | | | | |
|---|---|---|---|---|---|---|---|---|
| | Storage at 4° C. | | | | Storage at RT (~22° C.) | | | |
| | Time of storage (months) | | | | | | | |
| Protein | 0 | 9 | 11 | 12 | 0 | 9 | 11 | 12 |
| Thaumatin-II | | | | | | | | |
| Batch #5 | 98.0 | | | 96.15 | | | | |
| Batch #17 | 96.2 | | | 95.35 | 96.2 | | 94.85 | |
| Batch #31 | 99.4 | 96.15 | | | 99.4 | 94.55 | | |

Thaumatin protein purity (Thaumatin protein as percent of total protein) was maintained when dry protein powders were stored over prolonged periods. None of the samples of Thaumatin-II showed degradation fragments or aggregation upon storage at either 4° C. or at room temperature (˜22° C.).

Thaumatin-I and Thaumatin-II were stable during storage under the conditions indicated. The percent purity values shown in Table 2 are averages of two replicate analyses. E.g., Thaumatin-II, was found stable for 12 months when stored at 4° C., with less than 3% loss of purity over that storage time. Some batches of Thaumatin-II were also stable at room temperature (RT) for up to 11 months. Generally, cold storage (4° C.-10° C.) is expected to provide greater stability, hence enabling longer duration of product storage.

### Example 9: Thaumatin-I and Thaumatin-II Quality Control: Residual Alkaloid Content

As Thaumatin proteins are expressed in Nicotiana benthamiana, residual alkaloids, especially nicotine and anabasine in the final product should be reduced to acceptable levels during purification. According to Sisson & Severson (1990) who performed nicotine determination by gas chromatography (GC), green leaves of N. benthamiana contain on average 15.8 mg/g dry weight total alkaloids, most of it being nicotine (90.4%) and anabasine (8.4%). Accordingly, in our studies we assumed that in wet leaves (90% moisture) we would find ˜1.5 mg nicotine/g fresh weight plant material. Actual measurements of nicotine and anabasine (the most prominent pyridine alkaloids in Nicotiana) by HPLC-MS showed amounts in the same range as those published (Stephan et al. 2017). Nevertheless, in our studies we could show that the nicotine content is about 10-fold lower than published values: 123,667±59,181 ng/g fresh weight. The same holds true in analyzing the anabasine content 14,133±2,590 ng/g fresh weight. According to Sisson & Severson (1990), is present in Nicotiana benthamiana on the level of 9.3% of nicotine. This difference in alkaloid concentration is most likely due to differences in experimental conditions (e.g. plant growth or extraction conditions).

Alkaloid content was determined by HPLC/MS analysis as described in Stephan et al. 2017. The method has a LLOQ of ˜20 ng/mL (20 ppb) and linearity of 20-1,500 ng/mL (20-1,500 ppb).

Table 3 is a summary of results of Thaumatin-I and Thaumatin-II analysis, showing the nicotine and anabasine alkaloid content of purified protein powders. For Thaumatin-I, one batch of purified protein was analyzed. In case of Thaumatin-II, three independent protein batches were analyzed. These batches were produced non-sequentially. Approximately 6 ng and 13-15 ng of residual nicotine per mg of Thaumatin-I and Thaumatin-III proteins, respectively were detected. Residual anabasine level was in the range between 0.34 and 3.84 for both proteins.

Our data prove that protein purification procedure which we use results in efficient reduction of alkaloids to safe levels.

TABLE 3
Residual alkaloid impurities in N. benthamiana
-produced Thaumatin-II.

| | Residual alkaloid level in purified protein samples | | | | |
|---|---|---|---|---|---|
| | Nicotine | | Anabasine | | |
| | (ng/mg protein) | | (ng/mg protein) | | No. of |
| | Single | | Single | | analyzed |
| Protein | Batches | Mean ± SD | Batches | Mean ± SD | batches |
| Thaumatin-I | 5.94 | | 2.35 | | 1 |
| Thaumatin-II | 14.09 | 14.41 ± 0.67 | 3.37 | 2.52 ± 1.55 | 3 |
| | 13.78 | | <3.84 | | |
| | 15.37 | | 0.34 | | |

As shown by the results obtained for Thaumatin-II, there is high reproducibility and consistency among batches for the most prevalent alkaloids, nicotine and anabasine.

### Example 10: Sensory Studies: Thaumatin-II Detection Threshold

The goal of this study was to determine the threshold of detection (thaumatin presence) and threshold of sweetness (sweet taste) for Thaumatin-III produced using our plant-virus based expression system. Detection threshold is the lowest concentration of substance in medium at which it can be detected as being different compared to blank control (“I perceive something”). Recognition threshold is the lowest concentration of substance in a medium at which it can be recognized as sweet (“I perceive sweetness”) (Lawless and Heymann, 2010). This study was performed at Nomad Bioscience GmbH research facility in Halle (Saale), Germany.

A Study Design

This evaluation was performed using Forced-Choice Ascending Concentration Series Method of Limits according to standard practice E679-04 (Reapproved 2011) from ASTM International (American Society for Testing and Material). The subject material consisted of Thaumatin-II expressed in Nicotiana benthamiana and purified as described in Example 5.

Solutions of Thaumatin-II were prepared at concentrations ranging from 0.01-3 ppm. These solutions were analyzed in 1:1.8 dilution steps, resulting in evaluation of 10 different dilutions for determination of threshold of detection (thaumatin presence) and threshold of sweetness. All solutions and dilutions used Milli-Q water to prevent potential taste differences due to water quality. The same water was used as a blank (control).

Clean, commercially purchased disposable plastic beakers (0.2 L) were used and were identical across all solutions tested. The beakers were marked with 3-digit randomized blinded codes and filled with water or test samples by an experimenter in randomized order for each concentration. After tasting, the samples were ejected into a cup. Between samplings, panelists rinsed their mouth with mineral non-sparkling water for up to 60 sec for cleansing. If necessary, sampling of water biscuits was allowed to remove prior flavors from the mouth, followed by rinsing as above.

A total of 19 participants/panelists were involved in this study. Stock solutions ranged in concentration of Thaumatin-II from 0.0151 ppm (0.679 nM) to 3 ppm (134.6 nM). No stock sucrose solution was included in this study. After each set tested (3 beakers), panelists filled score sheets assessing the differences they perceived among the three beakers in each set. Background controls and sample solutions were scored according to instructions (0=no difference, 1=taste something, 2=clear sweet taste detected), with the opportunity for a descriptive entry for each sample. The results were analyzed to determine the Thaumatin-II detection and Thaumatin-II sweetness threshold concentrations.

Results with Thaumatin-II

The results of the Thaumatin-II detection threshold portion of this study are shown in Table 4. Analyses show a low recognition threshold for Thaumatin-II, namely, 26 nM or 0.59 ppm; (n=18).

The results of the Thaumatin-II sweetness detection threshold are shown in Table 6. The lowest individual detection threshold established by one panelist was 1.64 nM. Collective results for the panel (n=16) revealed a lower concentration for the detection of sweetness for Thaumatin-II, namely, 37 nM (0.83 ppm), compared to the previously published 50 nM (1.2 ppm) (Masuda et al. 2018).

The differences in determination of threshold concentrations could be explained by potential higher purity of the protein or due to the fact that Thaumatin-II was dialyzed against water and not against a buffer containing salt, which might have influenced the taste evaluation.

TABLE 4
Thaumatin-II sensory detection threshold.

| | Concentration in nM | | | | | | | | | | | |
|---|---|---|---|---|---|---|---|---|---|---|---|---|
| Panelist | 0.68 | 1.22 | 2.2 | 3.96 | 7.12 | 12.82 | 23.08 | 41.54 | 74.78 | 134.6 | BET | log (BET) |
| 1 | + | + | 0 | 0 | 0 | + | + | + | + | + | 9.55 | 0.98 |
| 2 | 0 | 0 | + | + | + | + | 0 | + | + | + | 30.96 | 1.49 |
| 3 | 0 | 0 | + | 0 | + | + | + | + | + | + | 5.31 | 0.72 |
| 4 | 0 | + | 0 | + | + | 0 | + | + | + | + | 17.203 | 1.236 |
| 5 | 0 | 0 | 0 | 0 | + | 0 | + | + | + | + | 17.203 | 1.236 |
| 6 | 0 | 0 | 0 | + | + | + | + | + | + | + | 2.95 | 0.47 |
| 7 | + | 0 | + | + | + | + | + | + | + | + | 1.64 | 0.214 |
| 8 | 0 | 0 | 0 | 0 | + | + | + | + | + | + | 5.31 | 0.72 |
| 9 | + | 0 | 0 | 0 | + | + | + | + | + | + | 5.31 | 0.72 |
| 10 | 0 | 0 | 0 | 0 | 0 | 0 | + | 0 | + | + | 55.734 | 1.75 |
| 12 | + | 0 | 0 | 0 | 0 | + | + | + | + | + | 9.56 | 3.09 |
| 13 | 0 | + | + | + | + | 0 | + | 0 | + | + | 55.734 | 1.75 |
| 14 | 0 | 0 | 0 | 0 | + | 0 | 0 | + | + | + | 30.96 | 5.56 |
| 15 | 0 | 0 | + | + | + | + | + | + | + | + | 1.64 | 0.214 |
| 16 | + | 0 | 0 | 0 | + | + | + | 0 | + | + | 55.734 | 1.75 |
| 17 | + | 0 | + | + | + | + | + | + | + | + | 1.64 | 0.214 |
| 18 | 0 | + | + | 0 | 0 | + | + | 0 | + | + | 55.734 | 1.75 |
| 19 | + | 0 | 0 | 0 | + | 0 | 0 | 0 | + | + | 55.734 | 1.75 |
| Prop. | 0.28 | 0.22 | 0.39 | 0.39 | 0.78 | 0.67 | 0.83 | 0.72 | 1 | 1 | Mean | 1.42 |
| Corr. | | | | | | | | | | | (log (BET)) | |

| 10\hat{ }1.42 | 26.303 nM |
|---|---|
| | (0.59 ppm) |
| | Data analysis from ascending 3-AFC methods: correct choices indicated by + and incorrect by 0. |
| | BET (Best estimate of individual threshold) is geometric mean of first correct trial with all subsequent trials correct and the previous (incorrect) trial. |

TABLE 5
Thaumatin-II sweetness detection threshold.

| | Concentration in nM | | | | | | | | | | | |
|---|---|---|---|---|---|---|---|---|---|---|---|---|
| Panelist | 0.68 | 1.22 | 2.2 | 3.96 | 7.12 | 12.82 | 23.08 | 41.54 | 74.78 | 134.6 | nM | ppm |
| 1 | | | | | | X | | | | | 12.823 | 0.29 |
| 2 | | | | | | | | X | | | 41.54 | 0.93 |
| 3 | | | | | | | | X | | | 41.54 | 0.93 |
| 4 | | | | | | | X | | | | 23.0796 | 0.51 |
| 5 | | | | | | | | X | | | 41.54 | 0.93 |
| 6 | | | | | | | | X | | | 41.54 | 0.93 |
| 7 | | | | | X | | | | | | 7.123 | 0.16 |
| 8 | | | | | | X | | | | | 12.823 | 0.29 |
| 9 | | | | | X | | | | | | 7.123 | 0.16 |
| 10 | No sweet taste detected | | | | | | | | | | | |
| 12 | | | | | | X | | | | | 12.823 | 0.29 |
| 13 | No sweet taste detected | | | | | | | | | | | |
| 14 | | | | | | | | X | | | 41.54 | 0.93 |
| 15 | | | | | | X | | | | | 12.823 | 0.29 |
| 16 | | | | | | | | | X | | 74.778 | 1.67 |
| 17 | | | | | | | | | X | | 74.778 | 1.67 |
| 18 | | | | | | | | | X | | 74.778 | 1.67 |
| 19 | | | | | | | | | X | | 74.778 | 1.67 |

Average: 37.21 ± 25.98 nM
0.83 ± 0.58 ppm
Detection of sweetness: concentration marked with X if answer was correct with all subsequent answers correct and the previous (incorrect) answer.

### Example 11: Substitution of Sucrose with Thaumatin-I and Thaumatin-II

This study had two objectives. The first objective was to identify Thaumatin-I solution formulations with similar sensory profiles (sweetness and sweet aftertaste) as the control (10% sucrose solution). The second objective was to identify Thaumatin-II solution formulations with similar sensory profiles (sweetness and sweet aftertaste) as the control (10% sucrose solution). The study was performed at the Department of Food Science & Technology, College of Agricultural and Environmental Sciences, University of Georgia, Griffin, Ga., USA.

A Study Design

A hybrid descriptive method combining difference from control technique and attribute intensity rating was used. Evaluations were done by six trained panelists. A 0 to 15-point scale with 0.5 increments was used. Prior the experiment, the intensity rating for the sweet control (10% sucrose) was performed. Unlike the sucrose, Thaumatin sweetness was building up more slowly and reaching the maximum after 5 seconds. Therefore, all attributes were rated after holding the sample in the mouth for 5 seconds. To evaluate the trend of sweet aftertaste, panelists evaluated the aftertaste every 20s for 2 minutes. The timing was controlled by the panel leader. The test was replicated five times.

Results

1. Thaumatin-I.

1.1 Sweetness. The sweetness intensity of the control solution (10% sucrose) was 9.0. Sample with 6% sucrose+3.5 ppm Thaumatin-I had similar sweetness intensity as the control (Table 6). Only 5% sucrose+3.5 ppm Thaumatin-I had lower sweetness intensity than the control.

TABLE 6
Mean sweetness intensity scores for the samples.

| Solutions | Sweetness (P ≤ 0.05) |
|---|---|
| 7% Sucrose + 7 ppm Thaumatin-I | 14.6a* ± 0.12 |
| 6% Sucrose + 7 ppm Thaumatin-I | 13.0b ± 0.05 |
| 5% Sucrose + 7 ppm Thaumatin-I | 11.4c ± 0.09 |
| 7% Sucrose + 3.5 ppm Thaumatin-I | 10.3d ± 0.12 |
| 6% Sucrose + 3.5 ppm Thaumatin-I | 9.0e ± 0.00 |
| Control (10% Sucrose) | 9.0e |
| 5% Sucrose + 3.5 ppm Thaumatin-I | 8.0f ± 0.13 |

*Different letters in this column (a, b, c, d, e, f) indicate significant difference (P < 0.05) as analyzed by ANOVA (analysis of variance).
Control = 10% sugar (sucrose) solution.

1.2. Sweet Aftertaste. Compared to sucrose, Thaumatin-I samples had a more lingering sweet aftertaste. The sweet aftertaste intensities of the control solution (10% sugar) were 6, 3, 2,1, 1, and 0.5 at 20, 40, 60, 80, 100, and 120s, respectively. Although the sample with 6% sugar+3.ppm Thaumatin-I had similar sweetness intensity as the control, It showed a different pattern (more lingering) for the sweet aftertaste (FIG. 14). All samples that contained Thaumatin-I had relatively higher sweet aftertaste intensities than the control at all time points (20s to 120). The sample with 5% sucrose+3.5 ppm Thaumatin-I had the sweet aftertaste pattern closest to the control (10% sucrose).

2. Thaumatin-II.

2.1 Sweetness. The sweetness intensity of the control solution (10% sucrose) was 9.0. Sample with 5% sucrose+5 ppm Thaumatin-II had similar sweetness intensity as the control (Table 7). Sample with 5% sucrose+3.5 ppm Thaumatin-I was the only one that had lower sweetness intensity than the control. Sweetness intensities of two samples (7% sucrose+7 ppm Thaumatin II, and 7% sucrose+9 ppm Thaumatin II) were perceived to be above 15, which was the maximum value on the scale that was used.

TABLE 7
Mean sweetness intensity scores for the samples.

| Solutions | Sweetness (P ≤ 0.05) |
|---|---|
| 7% Sucrose + 9 ppm Thaumatin-II | 15++ |
| 7% Sucrose + 7 ppm Thaumatin-II | 15+ |
| 6% Sucrose + 9 ppm Thaumatin-II | 14.4a* ± 0.48 |
| 6% Sucrose + 7 ppm Thaumatin-II | 13.0b ± 0.23 |
| 5% Sucrose + 9 ppm Thaumatin-II | 12.6b ± 0.06 |
| 6% Sucrose + 5 ppm Thaumatin-II | 11.5c ± 0.19 |
| 7% Sucrose + 5 ppm Thaumatin-II | 11.3c ± 0.35 |
| 5% Sucrose + 7 ppm Thaumatin-II | 10.7d ± 0.26 |
| 7% Sucrose + 3.5 ppm Thaumatin-II | 10.0e ± 0.04 |
| 6% Sucrose + 3.5 ppm Thaumatin-II | 9.9e ± 0.12 |
| Control (10% Sucrose) | 9.0f |
| 5% Sucrose + 5 ppm Thaumatin-II | 8.9f ± 0.11 |
| 5% Sucrose + 3.5 ppm Thaumatin-II | 7.9g ± 0.10 |

*Different letters in this column (a, b, c, d, e, f, g) indicate significant difference (P < 0.05) as analyzed by ANOVA (analysis of variance).

2.2 Sweet Aftertaste. Compared to sucrose, Thaumatin-II samples had a more lingering sweet aftertaste. The sweet aftertaste intensities of the control solution (10% sucrose) were 6, 3, 2, 1, 1, and 0.5 at 20, 40, 60, 80, 100, and 120s, respectively. Although sample with 5% sucrose+5 ppm Thaumatin-II had similar sweetness intensity as the control, it showed a different pattern (more lingering) for the sweet aftertaste (FIG. 15). All samples containing Thaumatin-II had relatively higher sweet aftertaste intensities than the control at all time points (20s to 120s). The sample with 5% sucrose+3.5 ppm Thaumatin-II had lower initial sweetness intensity if compared with control (7.9±0.10 vs 9.0), nevertheless their sweet aftertaste patterns were quite similar (FIG. 16).

3. Sweetness Comparison of Thaumatin-I to Thaumatin-II. In general, Thaumatin-I and Thaumatin-II were very similar in sweet taste as seen in FIG. 16. Thaumatin-II was characterized by mouth-coating and had a lingering aftertaste that persisted more than Thaumatin-I. In the same time, Thaumatin-I was characterized by some artificial, chemical, and astringent attributes. Thaumatin-I had less mouth-coating than Thaumatin-II and faded faster than the Thaumatin-II solutions as regards the aftertaste.

Considering observed undesirable taste attributes of Thaumatin-I (artificial, chemical, and astringent), Thaumatin-II is preferred over Thaumatin-I.

4. Regression equation of sugar with Thaumatin-II. Based on the sweetness data for samples containing sucrose and Thaumatin-II, the generalized linear model procedure in SAS (SAS Institute Inc. Cary, N.C.) was used to fit a linear regression model (Little et al. 2002). The regression equation obtained allows determining the amount of Thaumatin-III or sucrose to be used with a constant amount of sucrose or Thaumatin-II, respectively, to achieve the targeting sweetness.

Next, regression equation was obtained:

Sweetness=1.62+0.74×Sucrose+0.25×Thaumatin-II+0.11×Sucrose×Thaumatin-II

“Sucrose” is the content of the sugar sucrose in weight-%. “Thaumatin-II” is the content of Thaumatin-II in ppm (by weight).
If the targeting sweetness of the solution is =9 (the sweetness of 10% sucrose),

9=1.62+0.74×Sucrose+0.25×Thaumatin-II+0.11×Sucrose×Thaumatin-II

0.74×Sucrose+0.25×Thaumatin-II+0.11×Sucrose×Thaumatin-II=7.38

One has to keep either the sugar or the Thaumatin-II constant to determine the other.

Table 8 shows the amounts of Thaumatin-II which have to be mixed with certain amounts of sucrose to get the sweetness equivalent to the sweetness of 10% sucrose solution.

TABLE 8
Levels of Thaumatin-II and sucrose corresponding to sucrose
and Thaumatin-II concentrations, respectively.

| Sucrose | Thaumatin-II | Thaumatin-II | Sucrose |
|---|---|---|---|
| (%) | ( ppm) | ( ppm) | (%) |
| 8.0 | 1.3 | 1.0 | 8.4 |
| 7.5 | 1.7 | 1.5 | 7.7 |
| 7.0 | 2.2 | 2.0 | 7.2 |
| 6.5 | 2.7 | 2.5 | 6.7 |
| 6.0 | 3.2 | 3.0 | 6.2 |
| 5.5 | 3.9 | 3.5 | 5.8 |
| 5.0 | 4.6 | 4.0 | 5.4 |
| 4.5 | 5.4 | 4.5 | 5.1 |
| 4.0 | 6.4 | 5.0 | 4.8 |

Example 1: Sucrose at 5%
0.74 × 5 + 0.25 × Thaumatin-II + 0.11 × 5 × Thaumatin-II = 7.38
Thaumatin-II = (7.38 − 3.7)/0.8 = 4.6 ppm
Example 2: Thaumatin-II at 4 ppm
0.74 × Sucrose + 0.25 × 4 + 0.11 × Sucrose × 4 = 7.38
Sucrose = (7.38 − 1)/1.18 = 5.4%

### Example 12: Substitution of HFCS with Thaumatin-II

High-fructose corn syrup (HFCS) is a sweetener made from corn starch. HFCS-55 contains 23% (w/w) of water and 77% (w/w) of solids. Solids in turn consist of 55% (w/w) of fructose, 41-42% (w/w) of glucose and 3-4% (w/w) of glucose oligosaccharides. HFCS-55 was strategically designed to have the same relative sweetness as sucrose so it could be easily substituted for sucrose in foods and beverages (White, 2014).

The objective of this study was to identify Thaumatin-II concentration in water with equivalent or similar sensory profiles (sweetness and sweet aftertaste) as the control (HFCS-55 solution in relation to 10% sucrose solution in water). The study was performed at the Department of Food Science & Technology, College of Agricultural and Environmental Sciences, University of Georgia, Griffin, Ga., USA.

### Study Design

A hybrid descriptive method combining difference from control technique and attribute intensity rating was used. Evaluations were done by six trained panelists. The intensity ratings for the sweet controls, determined as described in Example 11 (Sweetness 9 for 10% sucrose solution), was used. The equivalent sweetness of HFCS-55 in relation to 10% sucrose solution (10° Brix), 0.13 g/ml HFCS-55 or 10% (w/w) HFCS-55 solids in water solution, was established and used in this study. Degrees Brix (symbol ° Brix) is the sucrose content of an aqueous solution. 1° Brix is 1 gram of sucrose in 100 grams of solution and represents the strength of the solution as percentage by mass. 1° Brix is of HFCS-55 is 1 gram of the solid content of HFCS-55 in 100 grams of solution.

In comparison to the sweetness control, intensity ratings of the samples were determined. A 0 to 15-point scale with 0.5 increments was used. Unlike sucrose, Thaumatin sweetness was building up more slowly and reaching the maximum after 5 seconds. Therefore, the sweetness was rated after holding the sample in the mouth for 5 seconds. To evaluate the trend of sweet aftertaste, panelists evaluated the aftertaste every 20 s for 2 minutes. The off-feel, dryness, was perceived by the panelists with samples with HFCS. To evaluate the dryness aftertaste, panelists evaluated the aftertaste at 20s after the expectoration. To remove the dryness of the sample after the evaluation, the palate cleansing procedure (crackers, 0.2% salt water and regular water with the usage of toothbrush in between if needed) was used. The timing was controlled by the panel leader. The test was replicated five times.

### Results

1. Sweetness. The sweetness intensity of the control solution (10% sucrose) was 9.0. All samples had significantly higher sweetness scores (P<0.05) compared to the sweetness control (Table 9). The HFCS-55 solution equivalent to 10% sucrose solution (10° Brix, 0.13 g/ml HFCS-55) had a sweetness of 10.2. For both 30% and 40% HFCS reduction samples, the samples with 2 ppm and 3.5 ppm Thaumatin-III had similar sweetness intensity as the HFCS solution. With 50% HFCS reduction, the sample with 5 ppm Thaumatin II showed the most similar sweetness to the HFCS solution.

TABLE 9
Mean sweetness intensity scores for the samples.

| | | Sweetness |
|---|---|---|
| Solutions | | (P ≤ 0.01) |
| 40% HFCS Reduction + 7 ppm Th-II | 6° Brix HFCS + 7 ppm Th-II | 11.1a* ± 0.29 |
| 30% HFCS Reduction + 5 ppm Th-II | 7° Brix HFCS + 5 ppm Th-II | 10.7b ± 0.11 |
| 40% HFCS Reduction + 5 ppm Th-II | 6° Brix HFCS + 5 ppm Th-II | 10.6bc ± 0.45 |
| 50% HFCS Reduction + 7 ppm Th-II | 5° Brix HFCS + 7 ppm Th-II | 10.6bc ± 0.29 |
| 30% HFCS Reduction + 3.5 ppm Th-II | 7° Brix HFCS + 3.5 ppm Th-II | 10.4cd ± 0.28 |
| 40% HFCS Reduction + 3.5 ppm Th-II | 6° Brix HFCS + 3.5 ppm Th-II | 10.3cd ± 0.20 |
| 50% HFCS Reduction + 5 ppm Th-II | 5° Brix HFCS + 5 ppm Th-II | 10.3cd ± 0.32 |
| HFCS control | 10° Brix HFCS | 10.2de ± 0.17 |
| 30% HFCS Reduction + 2 ppm Th-II | 7° Brix HFCS + 2 ppm Th-II | 9.9ef ± 0.40 |
| 40% HFCS Reduction + 2 ppm Th-II | 6° Brix HFCS + 2 ppm Th-II | 9.9ef ± 0.20 |
| 50% HFCS Reduction + 3.5 ppm Th-II | 5° Brix HFCS + 3.5 ppm Th-II | 9.8f ± 0.25 |
| Sucrose control | 10% Sucrose solution | 9.0g |

*Different letters in this column (a, b, c, d, e, f, g) indicate significant difference (P < 0.05) as analyzed by ANOVA (analysis of variance).
Th-II: Thaumatin-II; 1° Brix = 1% w/w of the HFCS solid content.

2. Regression equation of HFCS with Thaumatin-II. The generalized linear model procedure in SAS (SAS Institute Inc. Cary, N.C.) was used to fit a linear regression model (Little et al. 2002). The regression equation obtained allows determining the amount of Thaumatin-II or HFCS to be used with a constant amount of HFCS or Thaumatin-II, respectively, to achieve the targeting sweetness.

Next regression equation was obtained:

Sweetness=8.073+0.206×HFCS+0.149×Thaumatin-II+0.016×HFCS×Thaumatin-II

In this equation, “HFCS” is the concentration of HFCS in % solid content of HFCS in w/w or ° Brix, and “Thaumatin-II” is the concentration of Thaumatin-II in ppm.

If the targeting sweetness of the solution is 10° Brix,

10=8.073+0.206×HFCS+0.149×Thaumatin-II+0.016×HFCS×Thaumatin-II

is used with either constant HFCS or Thaumatin-II to determine the other.

Table 10 shows the amounts of Thaumatin-II which have to be mixed with certain amounts HFCS to get the sweetness equivalent to the sweetness of 10° Brix HFCS solution.

TABLE 10
Levels of Thaumatin-II and HFCS corresponding to HFCS
and Thaumatin-II concentrations, respectively.

| HFCS | Thaumatin-II | Thaumatin-II | HFCS |
|---|---|---|---|
| (° Brix) | (ppm) | (ppm) | (° Brix) |
| 8.0 | 1.0 | 1.0 | 8.0 |
| 7.5 | 1.4 | 1.5 | 7.4 |
| 7.0 | 1.9 | 2.0 | 6.8 |
| 6.5 | 2.3 | 2.5 | 6.3 |
| 6.0 | 2.8 | 3.0 | 5.8 |
| 5.5 | 3.4 | 3.5 | 5.4 |
| 5.0 | 3.9 | 4.0 | 4.9 |
| 4.5 | 4.5 | 4.5 | 4.5 |
| 4.0 | 5.2 | 5.0 | 4.1 |

1° Brix = 1% solid content of HFCS in w/w

Example 1: Using 6Brix HFCS, level of Thaumatin-II needed to make 10° Brix solution.
10=8.073+0.206×6+0.149×Thaumatin-II+0.016×6×Thaumatin-II
Thaumatin-11=2.8 ppm
   Example 2: Using 3 ppm Thaumatin-II, brix of HFCS needed to make 10° Brix solution.
10=8.073+0.206×HFCS+0.149×3+0.016×HFCS×3
HFCS=5.8° Brix

3. Sweet Aftertaste. Compared to sucrose control, Thaumatin samples had a more lingering sweet aftertaste. The sweet aftertaste intensities of the control solution (10% sucrose) were 6, 3, 2, 1, 1, and 0.5 at 20, 40, 60, 80, 100, and 120s, respectively. The sweet aftertaste values of the HFCS solution were 7.2, 3.7, 2.6, 1.3, 0.8, and 0.6 at 20, 40, 60, 80, 100, and 120s, respectively. The aftertaste of HFCS was higher than the sucrose control till 80s. All of the reduced HFCS with Thaumatin samples showed a similar trend of sweet aftertaste (Table 11; FIG. 17).

TABLE 11
Sweet aftertaste trend for blends of HFCS with Thaumatin-
II: mean sweetness intensity scores upon the time

| Seconds after expectoration | 0 | 20 | 40 | 60 | 80 | 100 | 120 |
|---|---|---|---|---|---|---|---|
| 7°Brix HFCS, 2 ppm Th-II | 9.9 | 7.6 | 4.9 | 3.3 | 2.0 | 1.4 | 0.9 |
| 7°Brix HFCS, 3.5 ppm Th-II | 10.3 | 7.6 | 4.8 | 3.1 | 2.0 | 1.4 | 1.0 |
| 7°Brix HFCS, 5 ppm Th-II | 10.7 | 8.0 | 5.3 | 3.6 | 2.3 | 1.6 | 1.1 |
| 6°Brix HFCS, 2 ppm Th-II | 9.9 | 7.4 | 4.8 | 3.3 | 1.8 | 1.2 | 0.7 |
| 6°Brix HFCS, 3.5 ppm Th-II | 10.3 | 7.7 | 5.0 | 3.2 | 21 | 1.5 | 1.0 |
| 6°Brix HFCS, 5 ppm Th-II | 10.6 | 7.7 | 5.1 | 3.4 | 2.0 | 1.5 | 1.0 |
| 6°Brix HFCS, 7 ppm Th-II | 11.1 | 8.1 | 5.6 | 3.9 | 2.4 | 1.8 | 1.2 |
| 5°Brix HFCS, 3.5 ppm Th-II | 9.8 | 7.3 | 4.8 | 3.0 | 1.7 | 1.1 | 0.7 |
| 5°Brix HFCS, 5 ppm Th-II | 10.3 | 7.6 | 5.0 | 3.4 | 2.0 | 1.6 | 1.1 |
| 5°Brix HFCS, 7 pm Th-II | 10.7 | 7.7 | 5.0 | 3.3 | 2.1 | 1.4 | 1.0 |
| 10% Sucrose | 9 | 6 | 3 | 2 | 1 | 1 | 0.5 |
| 10°Brix HFCS | 10 | 7.2 | 3.7 | 2.6 | 1.3 | 0.8 | 0.6 |

4. Dryness. The dryness was an off-feel noted with solutions containing HFCS. The dryness of the HFCS solution was 4. All of the reduced HFCS with added Thaumatin-I solutions showed a lower dryness level than the HFCS solution (Table 12). For 30% HFCS reduced samples, the dryness of the samples increased with the higher level of Thaumatin-I. There was no effect of Thaumatin level on dryness showed to the samples with 40% and 50% reduction of HFCS.

TABLE 12
Mean dryness intensity scores for the samples

| Solutions | | Dryness (P ≤ 0.01) |
|---|---|---|
| HFCS control | 10° Brix HFCS | 4.0a* |
| 40% HFCS Reduction + 2 ppm Th-II | 6° Brix HFCS + 2 ppm Th-II | 3.5bcd ± 0.24 |
| 40% HFCS Reduction + 5 ppm Tha-II | 6° Brix HFCS + 5 ppm Th-II | 3.5bc ± 0.17 |
| 40% HFCS Reduction + 7 ppm Th-II | 6° Brix HFCS + 7 ppm Th-II | 3.5b ± 0.10 |
| 30% HFCS Reduction + 5 ppm Th-II | 7° Brix HFCS + 5 ppm Th-II | 3.4bcd ± 0.12 |
| 50% HFCS Reduction + 5 ppm Th-II | 5° Brix HFCS + 5 ppm Th-II | 3.3bcde ± 0.25 |
| 50% HFCS Reduction + 7 ppm Th-II | 5° Brix HFCS + 7 ppm Th-II | 3.3bcd ± 0.14 |
| 30% HFCS Reduction + 3.5 ppm Th-II | 7° Brix HFCS + 3.5 ppm Th-II | 3.3cde ± 0.13 |
| 50% HFCS Reduction + 3.5 ppm Th-II | 5° Brix HFCS + 3.5 ppm Th-II | 3.2de ± 0.36 |
| 40% HFCS Reduction + 3.5 ppm Th-II | 6° Brix HFCS + 3.5 ppm Th-II | 3.2de ± 0.23 |
| 30% HFCS Reduction + 2 ppm Th-II | 7° Brix HFCS + 2 ppm Th-II | 3.1e ± 0.20 |
| Sucrose control | 10% Sucrose solution | — |

*Different letters in this column (a, b, c, d, e, f, g) indicate significant difference (P < 0.05) as analyzed by ANOVA (analysis of variance).

### Conclusions

The concentration of the HFCS-55 which is equivalent to the 10% sucrose solution (10° Brix) was 0.13 g/mL HFCS 55. This solution was 1.2 points sweeter than the control solution (Sweetness 9 on a 0 to 15 point scale). Unlike the sucrose solution, the HFCS solution had an off-feel (dryness of tongue) of 4.

The HFCS samples required more Thaumatin-II (3.5 ppm Thaumatin-II) than sucrose samples (2 ppm Thaumatin-II) with 30-40% reduction. With 50% reduction, however, the amount of Thaumatin-II required for equivalent sweetness intensity was similar for both HFCS and sucrose samples. When 3.5 ppm Thaumatin-II was added, the increase of sweetness of the samples with HFCS was higher than sucrose samples. However, with 5 ppm or 7 ppm Thaumatin-II addition, the increase of the sweetness intensity was higher for the samples with sucrose.

The dryness was a distinctive off-feel noted with HFCS solutions. As the amount of HFCS is reduced, the dryness level is also decreased. With the addition of the Thaumatin-II to those HFCS-reduced samples, the dryness of the samples increased, but was lower than that of the HFCS control.

### Example 13: Preparation of Sweet Fruit Lemonade with Reduced Carbohydrate Content

Sweet soft drinks typically contain about 10% sugar or an equivalent amount of HFCS. On average, 100% fruit juice also contains about 10% sugars.

To prepare 1 liter of fruit lemonade with the same sweetness as a soft drink containing 10% sucrose, but with 50% reduced sucrose content, one may mix together 100 ml of filtered 100% fruit juice, 1.5 g citric acid, 40 g sugar, 4.6 mg Thaumatin-II, and adjust the volume to 1 liter with mineral water. Juices from various fruits can be used alone or in blends: orange, mandarin, apple, pear, cherry, raspberry, cranberry, blackcurrant, plums, etc. Optionally, lemonade can be carbonated by injecting pressurized carbon dioxide.

To prepare 1 liter of fruit lemonade with same sweetness as a soft drink containing 10% (w/v) of HFCS-55, but with 50% reduced HFCS-55 content, one may mix together 100 ml of filtered 100% fruit juice, 1.5 g citric acid, 40 g HFCS-55, 3.9 mg Thaumatin-II, and adjust the volume to 1 liter with mineral water. Juices from various fruits can be used alone or in blends: orange, mandarin, apple, pear, cherry, raspberry, cranberry, blackcurrant, plums, etc. Optionally, lemonade can be carbonated by injecting pressurized carbon dioxide.

### REFERENCES

1. Engler C, Gruetzner R, Kandzia R, Marillonnet S (2009) Golden Gate Shuffling: A One-Pot DNA Shuffling Method Based on Type IIs Restriction Enzymes. PLOS One 4, e5553.
2. Hahn S, Giritch A, Bartels D, Bortesi L, Gleba Y (2015) A novel and fully scalable Agrobacterium spray-based process for manufacturing cellulases and other cost-sensitive proteins in plants. Plant Biotechnol J 13(5): 708-716.
3. Horsch R B, Fry J E, Hoffmann N L, Eichholtz D, Rogers S G, Fraley R T (1985) A simple and general method for transferring genes into plants. Science 227: 1229-1231.
4. Lawless H T and Heymann H (2010) Sensory Evaluation of Food. Principles and Practices. 2^{nd} ed. Springer: New York, Dordrecht, Heidelberg, London. 596 p.
5. Littell R C, Stroup W W, Freund R J. 2002. Regression. SAS for Linear Models, Fourth Edition, pp 3-32, SAS Institute Inc., Cary, N.C.
6. Marillonnet S, Thoeringer C, Kandzia R, Klimyuk V, Gleba Y (2005) Systemic Agrobacterium Tumefaciens-Mediated Transfection of Viral Replicons for Efficient Transient Expression in Plants. Nat Biotechnol 23(6): 718-723.
7. Masuda T, Ohta K, Ojiro N, Murata K, Mikami B, Tani F, Temussi P A, Kitabatake N (2016) A Hypersweet Protein: Removal of The Specific Negative Charge at Asp21 Enhances Thaumatin Sweetness. Sci Rep 6:20255.
8. Sisson V A, Severson R F (1990) Alkaloid composition of the Nicotiana species. Beitr Tabakforsch 14: 327-339.
9. Stephan A, Hahn-Löbmann S, Rosche F, Buchholz M, Giritch A, Gleba Y (2017) Simple Purification of Nicotiana benthamiana-Produced Recombinant Colicins: High-Yield Recovery of Purified Proteins With Minimum Alkaloid Content Supports the Suitability of the Host for Manufacturing Food Additives. Int J Mol Sci 9(1): 95.
10. Weber E, Engler C, Gruetzner R, Werner S, Marillonnet S (2011) A modular cloning system for standardized assembly of multigene constructs. PLoS One 6(2): e16765.
11. Werner S, Breus O, Symonenko Y, Marillonnet S, Gleba Y (2011) High-level Recombinant Protein Expression in Transgenic Plants by Using a Double-Inducible Viral Vector. Proc Natl Acad Sci USA 108(34): 14061-14066.
12. White J S (2014) Sucrose, HFCS, and Fructose: History, Manufacture, Composition, Applications, and Production. In: Rippe J. (eds) Fructose, High Fructose Corn Syrup, Sucrose and Health. Nutrition and Health. Humana Press, New York, N.Y.

### Nucleotide and Amino Acid Sequences

Amino acid sequence of Thaumatococcus daniellii
preprothaumatin-I (GenBank: BAF44567.1)
SEQ ID NO: 1
MAATTCFFFLFPFLLLLTLSRAATFEIVNRCSYTVWAAASKGDAALDAGGRQLNSGESWTINVEPGTNG
GKIWARTDCYFDDSGSGICKTGDCGGLLRCKRFGRPPTTLAEFSLNQYGKDYIDISNIKGFNVPMDFSP
TTRGCRGVRCAADIVGQCPAKLKAPGGGCNDACTVFQTSEYCCTTGKCGPTEYSRFFKRLCPDAFSYVL
DKPTTVTCPGSSNYRVTFCPTALELEDE
Nucleotide sequence of Thaumatococcus daniellii mRNA for
preprothaumatin-I, complete cds (GenBank: AB265690.1)
SEQ ID NO: 2
atggccgccaccacttgcttcttcttcctcttccccttcctcctcctcctcacgctctcccgcgctgcc
accttcgagatcgtcaaccgctgctcctacaccgtgtgggcggccgcctccaaaggcgacgccgccctg
gacgccggcggccgccagctcaactcgggagagtcctggaccatcaacgtagaacccggcaccaacggt
ggcaaaatctgggcccgcaccgactgctatttcgacgacagcggcagcggcatctgcaagaccggcgac
tgcggcggcctcctccggtgcaagcgcttcggccggccgcccaccacgctggcggagttctcgctcaac
cagtacggcaaggactacategacatctccaacatcaaaggcttcaacgtgccgatggacttcagcccg
accacgcgcggctgccgcggggtgcggtgcgccgccgacatcgtggggcagtgcccggcgaagctgaag
gcgccggggggggttgcaacgatgcgtgcaccgtgttccagacgagcgagtactgctgcaccacgggga
agtgcgggccgacggagtactcgcgcttcttcaagaggctttgcccggacgcgttcagttatgtcctgg
acaagccaaccaccgtcacctgccccggcagctccaactacagggtcactttctgccctactgcccttg
aacttgaagacgagtaa
Amino acid sequence of Thaumatococcus daniellii
preprothaumatin-II (GenBank: AAA93095.1)
SEQ ID NO: 3
MAATTCFFFLFPFLLLLTLSRAATFEIVNRCSYTVWAAASKGDAALDAGGRQLNSGESWTINVEPGTKG
GKIWARTDCYFDDSGRGICRTGDCGGLLQCKRFGRPPTTLAEFSLNQYGKDYIDISNIKGFNVPMDFSP
TTRGCRGVRCAADIVGQCPAKLKAPGGGCNDACTVFQTSEYCCTTGKCGPTEYSRFFKRLCPDAFSYVL
DKPTTVTCPGSSNYRVTFCPTALELEDE
Nucleotide sequence of Thaumatococcus daniellii mRNA for
preprothaumatin-II, complete cds (GenBank: J01209.1)
SEQ ID NO: 4
Atggccgccaccacttgcttcttcttcctcttccccttcctcctcctcctcacgctctcccgcgctgcc
accttcgagatcgtcaaccgctgctcctacaccgtgtgggcggccgcctccaaaggcgacgccgccctg
gacgccggcggccgccagctcaactcgggagagtcctggaccatcaacgtagaacccggcaccaagggt
ggcaaaatctgggcccgcaccgactgctatttcgacgacagcggccgcggcatctgccggaccggcgac
tgcggcggcctcctccagtgcaagcgcttcggccggccgcccaccacgctggcggagttctcgctcaac
cagtacggcaaggactacatcgacatctccaacatcaaaggcttcaacgtgccgatggacttcagcccg
accacgcgcggctgccgcggggtgcggtgcgccgccgacatcgtggggcagtgcccggcgaagctgaag
gcgccggggggggttgcaacgatgcgtgcaccgtgttccagacgagcgagtactgctgcaccacgggga
agtgcgggccgacggagtactcgcgcttcttcaagaggctttgcccggacgcgttcagttatgtcctgg
acaagccaaccaccgtcacctgccccggcagctccaactacagggtcactttctgccctactgcccttg
aacttgaagacgagtaa
Amino acid sequence of Thaumatococcus daniellii
Thaumatin-I mature protein (GenBank: AAL83964.1)
SEQ ID NO: 5
ATFEIVNRCSYTVWAAASKGDAALDAGGRQLNSGESWTINVEPGTNGGKIWARTDCYFDDSGSGICKTG
DCGGLLRCKRFGRPPTTLAEFSLNQYGKDYIDISNIKGFNVPMDFSPTTRGCRGVRCAADIVGQCPAKL
KAPGGGCNDACTVFQTSEYCCTTGKCGPTEYSRFFKRLCPDAFSYVLDKPTTVTCPGSSNYRVTFCPTA
Amino acid sequence of Thaumatococcus daniellii
Thaumatin-II mature protein (GenBank: AAA93095.1)
SEQ ID NO: 6
ATFEIVNRCSYTVWAAASKGDAALDAGGRQLNSGESWTINVEPGTKGGKIWARTDCYFDDSGRGICRTG
DCGGLLQCKRFGRPPTTLAEFSLNQYGKDYIDISNIKGFNVPMDFSPTTRGCRGVRCAADIVGQCPAKL
KAPGGGCNDACTVFQTSEYCCTTGKCGPTEYSRFFKRLCPDAFSYVLDKPTTVTCPGSSNYRVTFCPTA
Nucleotide sequence of mature-protein-encoding
fragment of Thaumatococcus daniellii mRNA for
preprothaumatin-I (GenBank: AF355098.1)
SEQ ID NO: 7
gccaccttcgagatcgtcaaccgctgctcctacaccgtgtgggcggccgcctccaaaggcgacgccgcc
ctggacgccggcggccgccagctcaactcgggagagtcctggaccatcaacgtagaacccggcaccaac
ggtggcaaaatctgggcccgcaccgactgctatttogacgacagcggcagcggcatctgcaagaccggc
gactgcggcggcctcctccggtgcaagcgcttcggccggccgcccaccacgctggcggagttctcgctc
aaccagtacggcaaggactacatcgacatctccaacatcaaaggcttcaacgtgccgatggacttcagc
ccgaccacgcgcggctgccgcggggtgcggtgcgccgccgacatcgtggggcagtgcccggcgaagctg
aaggcgccggggggggttgcaacgatgcgtgcaccgtgttccagacgagcgagtactgctgcaccacgg
ggaagtgcgggcogacggagtactcgcgcttcttcaagaggctttgcccggacgcgttcagttatgtcc
tggacaagccaaccaccgtcacctgccccggcagctccaactacagggtcactttctgccctactgcc
Nucleotide sequence of mature-protein-encoding
fragment of Thaumatococcus daniellii mRNA for
preprothaumatin-II (GenBank: J01209.1)
SEQ ID NO: 8
Gccaccttcgagatcgtcaaccgctgctcctacaccgtgtgggcggccgcctccaaaggcgacgccgcc
ctggacgccggcggccgccagctcaactcgggagagtcctggaccatcaacgtagaacccggcaccaag
ggtggcaaaatctgggcccgcaccgactgctatttcgacgacagcggccgcggcatctgccggaccggc
gactgcggcggcctcctccagtgcaagcgcttcggccggccgcccaccacgctggcggagttctcgctc
aaccagtacggcaaggactacatcgacatctccaacatcaaaggcttcaacgtgccgatggacttcagc
ccgaccacgcgcggctgccgcggggtgcggtgcgccgccgacatcgtggggcagtgcccggcgaagctg
aaggcgccggggggtggttgcaacgatgcgtgcaccgtgttccagacgagcgagtactgctgcaccacg
gggaagtgcgggccgacggagtactcgcgcttcttcaagaggctttgcccggacgcgttcagttatgtc
ctggacaagccaaccaccgtcacctgccccggcagctccaactacagggtcactttctgccctactgcc
Nucleotide sequence of translational fusion of
N-terminal apoplast targeting presequence from
Oryza sativa RAmy3A gene for alpha-amylase and
mature Thaumatin-I
SEQ ID NO: 9
Atggggaagcaaatggccgccctgtgtggctttctcctcgtggcgttgctctggctcacgcccgacgtc
gcgtcaggtgccaccttcgagatcgtcaaccgctgctcctacaccgtgtgggcggccgcctccaaaggc
gacgccgccctggacgccggcggccgccagctcaactcgggagagtcctggaccatcaacgtagaaccc
ggcaccaacggtggcaaaatctgggcccgcaccgactgctatttcgacgacagcggcagcggcatctgc
aagaccggcgactgcggcggcctcctccggtgcaagcgcttcggccggccgcccaccacgctggcggag
ttctcgctcaaccagtacggcaaggactacatcgacatctccaacatcaaaggcttcaacgtgccgatg
gacttcagcccgaccacgcgcggctgccgcggggtgcggtgcgccgccgacatcgtggggcagtgcccg
gcgaagctgaaggcgccggggggtggttgcaacgatgcgtgcaccgtgttccagacgagcgagtactgc
tgcaccacggggaagtgcgggccgacggagtactcgcgcttcttcaagaggctttgcccggacgcgttc
agttatgtcctggacaagccaaccaccgtcacctgccccggcagctccaactacagggtcactttctgc
cctactgcctaa
Nucleotide sequence of translational fusion of
N-terminal apoplast targeting presequence from
Oryza sativa RAmy3A gene for alpha-amylase and
mature Thaumatin-II
SEQ ID NO: 10
atggggaagcaaatggccgccctgtgtggctttctcctcgtggcgttgctctggctcacgcccgacgtc
gcgtcaggtgccaccttcgagatcgtcaaccgctgctcctacaccgtgtgggcggccgcctccaaaggc
gacgccgccctggacgccggcggccgccagctcaactcgggagagtcctggaccatcaacgtagaaccc
ggcaccaagggtggcaaaatctgggcccgcaccgactgctatttcgacgacagcggccgcggcatctgc
cggaccggcgactgcggcggcctcctccagtgcaagcgcttcggccggccgcccaccacgctggcggag
ttctcgctcaaccagtacggcaaggactacatcgacatctccaacatcaaaggcttcaacgtgccgatg
gacttcagcccgaccacgegcggctgccgcggggtgcggtgcgccgccgacatcgtggggcagtgcccg
gcgaagctgaaggcgccggggggggttgcaacgatgcgtgcaccgtgttccagacgagcgagtactgct
gcaccacggggaagtgcgggccgacggagtactcgcgcttcttcaagaggctttgcccggacgcgttca
gttatgtcctggacaagccaaccaccgtcacctgccccggcagctccaactacagggtcactttctgcc
ctactgcctaa
Nucleotide sequence of T-DNA region of pNMD40502
SEQ ID NO: 11
The sequence is provided in the sequence listing.
Nucleotide sequence of T-DNA region of pICH95397
SEQ ID NO: 12
The sequence is provided in the sequence listing.
Nucleotide sequence of T-DNA region of pNMD40523
SEQ ID NO: 13
The sequence is provided in the sequence listing.
Nucleotide sequence of T-DNA region of pNMD38061
SEQ ID NO: 14
The sequence is provided in the sequence listing.

This patent application claims priority of the European Patent Application No. 20186323.0 filed on Jul. 16, 2020, the entirety of which is incorporated by reference herein including description, all claim, sequence listing, and figures.

1. A product for oral consumption, such as a food, comprising at least one thaumatin selected from the group consisting of thaumatin I and thaumatin II and at least one sugar selected from the group consisting of sucrose, glucose, and fructose in a mass ratio of said thaumatin:to said sugar of
from 1:2,000 to 1:80,000,
preferably from 1:4,000 to 1:65,000,
more preferably from 1:6,000 to 1:55,000,
even more preferably from 1:8,000 to 1:45,000, and
even more preferably from 1:10,000 to 1:35,000.

2. The product for oral consumption according to claim 1, comprising, with respect to the total weight of the product, 2 to 12 weight %, preferably from 3 to 10 weight %, more preferably from 4 to 8 weight % of said sugar selected from the group consisting of sucrose, glucose, and fructose; and/or
comprising thaumatin in a range from 1 to 13 ppm per total weight of the product, preferably from 3 ppm to 7 ppm, wherein the thaumatin is at least one thaumatin selected from the group consisting of thaumatin I and thaumatin II.

3. The product for oral consumption according to claim 1, comprising, with respect to the total weight of the product, 2 to 12 weight %, preferably from 3 to 10 weight %, more preferably from 4 to 8 weight % of said sugar selected from the group consisting of sucrose, glucose, and fructose; and
comprising thaumatin in a range from 1 to 13 ppm per total weight of the product, preferably from 3 ppm to 7 ppm, wherein the thaumatin is at least one thaumatin selected from the group consisting of thaumatin I and thaumatin II.

4. A product for oral consumption, such as a food, comprising at least one thaumatin selected from the group consisting of thaumatin I and thaumatin II and at least one sugar selected from the group consisting of sucrose, glucose, and fructose in a mass ratio of said thaumatin:to said sugar of 1:10,000 to 1:35,000 and
further comprising, with respect to the total weight of the product, 4 to 8 weight % of said sugar selected from the group consisting of sucrose, glucose, and fructose and
thaumatin in a range from 3 ppm to 7 ppm per total weight of the product, wherein the thaumatin is at least one thaumatin selected from the group consisting of thaumatin I and thaumatin II.

5. A product for oral consumption comprising thaumatin in a range from 1 to 13 ppm, preferably from 3 ppm to 7 ppm, per total weight of the product and at least one sugar selected from the group consisting of sucrose, glucose, and fructose, wherein the thaumatin is at least one thaumatin selected from the group consisting of thaumatin I and thaumatin II.

6. The product for oral consumption according to claim 1, comprising, with respect to the total weight of the product, 2 to 12 weight %, preferably from 3 to 10 weight %, more preferably from 4 to 8 weight % of said sugar selected from the group consisting of sucrose, glucose, and fructose.

7. The product for oral consumption according to claim 1, wherein said at least one sugar is selected from the group consisting glucose and fructose, said product not containing sucrose.

8. The product for oral consumption according to claim 1, said product comprising High Fructose Corn Syrup (HFCS) containing at least one sugar selected from the group consisting of glucose and fructose.

9. The product for oral consumption according to claim 8, wherein the HFCS is HFCS-42, HFCS-55, HFCS-65, HFCS-70 or HFCS-90.

10. The product for oral consumption according to claim 8, wherein the HFCS contains, per total weight of the HFCS, 22 to 25 weight % water and 78% to 75% dissolved or dispersed solids,
wherein said solids contain 15 to 92 weight % fructose, 8 to 85 weight % glucose, and 0 to 7 weight % glucose oligosaccharides per total weight of the solids, preferably said solids contain 40 to 65 weight % fructose, 30 to 55 weight % glucose, and 0 to 7 weight % glucose oligosaccharides per total weight of the solids.

11-13. (canceled)

14. The product for oral consumption according to claim 1, said product comprising High Fructose Corn Syrup (HFCS) as said at least one sugar selected from the group consisting of glucose and fructose.

15. The product for oral consumption according to claim 1 wherein the product is a beverage, beverage powder, drink, soft drink, yoghurt, jam, marmalade, beverage concentrate such as syrup, dessert, cake, biscuit, cookie, chocolate, candy, sweets, sugar confectionary, chewing gum, custard, pudding, jelly, filling jelly, pastry, pie, drops, processed foods, cereals, baked goods, or a medicament; wine or beer or other fermented or distilled beverages, potato-based snacks, breakfast cereals, chewing gum, ice cream, cocoa and chocolate products, breath mints, sugar decorations or icings, coatings or fillings, fine bakery items, food supplements or table-top sweeteners.

16. A composition suitable for producing a product for oral consumption, the composition comprising at least one thaumatin selected from the group consisting of thaumatin I and thaumatin II and at least one sugar selected from the group consisting of sucrose, glucose, and fructose.

17. The composition according to claim 16, containing at least one sugar selected from the group consisting of glucose and fructose, said composition not containing sucrose.

18. (canceled)

19. The composition according to claim 16, containing said at least one thaumatin in a range of from 1 to 13 ppm per total weight of the composition, preferably from 3 ppm to 7 ppm per total weight of the composition.

20. The composition according to claim 16, containing said at least one thaumatin and said at least one sugar in a mass ratio of thaumatin:sugar of
from 1:2,000 to 1:80,000,
preferably from 1:4,000 to 1:65,000,
more preferably from 1:6,000 to 1:55,000,
even more preferably from 1:8,000 to 1:45,000, and
even more preferably from 1:10,000 to 1:35,000.

21. The composition according to claim 1, comprising said at least one thaumatin in a range of from 10 to 100 ppm per total weight of the composition and said at least one sugar in a range from 30 to 99.9%, preferably from 40 to 98%, more preferably from 50 to 90%, and even more preferably from 60 to 80% by weight per total weight of the composition.

22. The composition according to claim 16, comprising said at least one thaumatin in a range from 30 to 70 ppm per total weight of the composition and said at least one sugar in a range from 30 to 99.9%, preferably from 40 to 98%, more preferably from 50 to 90%, and even more preferably from 60 to 80% by weight per total weight of the composition.

23. The composition according to claim 16, comprising a further component for said product for oral consumption, said component being one or more component(s) selected from the group consisting of citric acid or a salt thereof, a vitamin, an inorganic salt, a trace element, caffeine, taurine, a gelling or thickening agent, a flavoring agent, and a preservative.

24. The composition according to claim 23, wherein said vitamin is one or more selected from ascorbic acid or a salt thereof, a vitamin B family vitamin, or tocopherol or a derivative thereof; said inorganic salt is selected from a sodium salt, a magnesium salt, a potassium salt, and a calcium salt; and/or said trace element is a zinc compound, an iron compound, or a copper compound.

25-26. (canceled)

27. The product or composition according to claim 1, wherein said thaumatin is thaumatin II.

28. A use of a composition as defined in claim 16 as a sweetening composition.

29. A use of a composition as defined in claim 16 for preparing a product for oral consumption.

30-34. (canceled)

35. A method of producing a product for oral consumption, comprising mixing the composition according to claim 16 with other components to produce said product.

36-41. (canceled)

42. The product according to claim 1, wherein said thaumatin is a protein comprising a polypeptide the amino acid sequence of which is that of SEQ ID NO: 5 or SEQ ID NO: 6, or an amino acid sequence having from 1 to 3 amino acid substitutions, additions, deletions, and/or insertions in the amino acid sequence of SEQ ID NO: 5 or 6, said thaumatin is preferably extracted from Nicotiana-species.

43. (canceled)


SEQUENCE LISTING (14 sequences)

SEQ ID NO: 1 - protein, 235 aa, Unknown
  Amino acid sequence of Thaumatococcus daniellii preprothaumatin-I(GenBank BAF44567.1)
MAATTCFFFLFPFLLLLTLSRAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTALELEDE

SEQ ID NO: 2 - DNA, 708 bp, Unknown
  Nucleotide sequence of Thaumatococcus daniellii mRNA for preprothaumatin-I, complete cds (GenBank AB265690.1)
atggccgccaccacttgcttcttcttcctcttccccttcctcctcctcctcacgctctcccgcgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgagtaa

SEQ ID NO: 3 - protein, 235 aa, Unknown
  Amino acid sequence of Thaumatococcus daniellii preprothaumatin-II (GenBank AAA93095.1)
MAATTCFFFLFPFLLLLTLSRAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTALELEDE

SEQ ID NO: 4 - DNA, 708 bp, Unknown
  Nucleotide sequence of Thaumatococcus daniellii mRNA for preprothaumatin-II, complete cds (GenBank J01209.1)
atggccgccaccacttgcttcttcttcctcttccccttcctcctcctcctcacgctctcccgcgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgagtaa

SEQ ID NO: 5 - protein, 207 aa, Unknown
  Amino acid sequence of Thaumatococcus daniellii Thaumatin-I mature protein (GenBank AAL83964.1)
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 6 - protein, 207 aa, Unknown
  Amino acid sequence of Thaumatococcus daniellii Thaumatin-II mature protein (GenBank AAA93095.1)
ATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 7 - DNA, 621 bp, Unknown
  Nucleotide sequence of mature-protein-encoding fragment of Thaumatococcus daniellii mRNA for preprothaumatin-I (GenBank AF355098.1)
gccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcc

SEQ ID NO: 8 - DNA, 621 bp, Unknown
  Nucleotide sequence of mature-protein-encoding fragment of Thaumatococcus daniellii mRNA for preprothaumatin-II (GenBank J01209.1)
gccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcc

SEQ ID NO: 9 - DNA, 702 bp, Unknown
  Nucleotide sequence of translational fusion of N-terminal apoplast targeting presequence from Oryza sativa RAmy3A gene for alpha-amylase and mature Thaumatin-I
atggggaagcaaatggccgccctgtgtggctttctcctcgtggcgttgctctggctcacgcccgacgtcgcgtcaggtgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcctaa

SEQ ID NO: 10 - DNA, 702 bp, Unknown
  Nucleotide sequence of translational fusion of N-terminal apoplast targeting presequence from Oryza sativa RAmy3A gene for alpha-amylase and mature Thaumatin-II
atggggaagcaaatggccgccctgtgtggctttctcctcgtggcgttgctctggctcacgcccgacgtcgcgtcaggtgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcctaa

SEQ ID NO: 11 - DNA, 10326 bp, Unknown
  Nucleotide sequence of T-DNA region of pNMD40502
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctagcttggaattggtaccacgcgtttcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaagttttagttttattgcaacaacaacaacaaattacaataacaacaaacaaaatacaaacaacaacaacatggcacaatttcaacaaacaattgacatgcaaactctccaagccgctgcgggacgcaacagcttggtgaatgatttggcatctcgtcgcgtttacgataatgcagtcgaggagctgaatgctcgttccagacgtcccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcaggtccacttctccaaggcagtgtctacggaacagaccctgattgcaacaaacgcatatccggagttcgagatttcctttactcatacgcaatccgctgtgcactccttggccggaggccttcggtcacttgagttggagtatctcatgatgcaagttccgttcggttctctgacgtacgacatcggcggtaacttttccgcgcaccttttcaaagggcgcgattacgttcactgctgcatgcctaatctggatgtacgtgacattgctcgccatgaaggacacaaggaagctatttacagttatgtgaatcgtttgaaaaggcagcagcgtcctgtgcctgaataccagagggcagctttcaacaactacgctgagaacccgcacttcgtccattgcgacaaacctttccaacagtgtgaattgacgacagcgaatggcactgacacctacgctgtagctctccatagcatttatgatatccctgttgaggagttcggttctgcgctactcaggaagaatgtgaaaacttgtttcgcggcctttcatttccatgagaatatgcttctagattgtgatacagtcacactcgatgagattggagctacgttccagaaatcaggtaacattccttagttacctttcttttctttttccatcataagtttatagattgtacatgctttgagatttttctttgcaaacaatctcaggtgataacctgagcttcttcttccataatgagagcactctcaattacacccacagcttcagcaacatcatcaagtacgtgtgcaagacgttcttccctgctagtcaacgcttcgtgtaccacaaggagttcctggtcactagagtcaacacttggtactgcaagttcacgagagtggatacgttcactctgttccgtggtgtgtaccacaacaatgtggattgcgaagagttttacaaggctatggacgatgcgtggcactacaaaaagacgttagcaatgcttaatgccgagaggaccatcttcaaggataacgctgcgttaaacttctggttcccgaaggtgctcttgaaattggaagtcttcttttgttgtctaaacctatcaatttctttgcggaaatttatttgaagctgtagagttaaaattgagtcttttaaacttttgtaggtgagagacatggttatcgtccctctctttgacgcttctatcacaactggtaggatgtctaggagagaggttatggtgaacaaggacttcgtctacacggtcctaaatcacatcaagacctatcaagctaaggcactgacgtacgcaaacgtgctgagcttcgtggagtctattaggtctagagtgataattaacggtgtcactgccaggtaagttgttacttatgattgttttcctctctgctacatgtattttgttgttcatttctgtaagatataagaattgagttttcctctgatgatattattaggtctgaatgggacacagacaaggcaattctaggtccattagcaatgacattcttcctgatcacgaagctgggtcatgtgcaagatgaaataatcctgaaaaagttccagaagttcgacagaaccaccaatgagctgatttggacaagtctctgcgatgccctgatgggggttattccctcggtcaaggagacgcttgtgcgcggtggttttgtgaaagtagcagaacaagccttagagatcaaggttagtatcatatgaagaaatacctagtttcagttgatgaatgctattttctgacctcagttgttctcttttgagaattatttcttttctaatttgcctgatttttctattaattcattaggttcccgagctatactgtaccttcgccgaccgattggtactacagtacaagaaggcggaggagttccaatcgtgtgatctttccaaacctctagaagagtcagagaagtactacaacgcattatccgagctatcagtgcttgagaatctcgactcttttgacttagaggcgtttaagactttatgtcagcagaagaatgtggacccggatatggcagcaaaggtaaatcctggtccacacttttacgataaaaacacaagattttaaactatgaactgatcaataatcattcctaaaagaccacacttttgttttgtttctaaagtaatttttactgttataacaggtggtcgtagcaatcatgaagtcagaattgacgttgcctttcaagaaacctacagaagaggaaatctcggagtcgctaaaaccaggagaggggtcgtgtgcagagcataaggaagtgttgagcttacaaaatgatgctccgttcccgtgtgtgaaaaatctagttgaaggttccgtgccggcgtatggaatgtgtcctaagggtggtggtttcgacaaattggatgtggacattgctgatttccatctcaagagtgtagatgcagttaaaaagggaactatgatgtctgcggtgtacacagggtctatcaaagttcaacaaatgaagaactacatagattacttaagtgcgtcgctggcagctacagtctcaaacctctgcaaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcttagagatgttcacggcgttgacccagagtcacaggagaaatctggagtgtgggatgttaggagaggacgttggttacttaaacctaatgcgaaaagtcacgcgtggggtgtggcagaagacgccaaccacaagttggttattgtgttactcaactgggatgacggaaagccggtttgtgatgagacatggttcagggtggcggtgtcaagcgattccttgatatattcggatatgggaaaacttaagacgctcacgtcttgcagtccaaatggtgagccaccggagcctaacgccaaagtaattttggtcgatggtgttcccggttgtggaaaaacgaaggagattatcgaaaaggtaagttctgcatttggttatgctccttgcattttaggtgttcgtcgctcttccatttccatgaatagctaagattttttttctctgcattcattcttcttgcctcagttctaactgtttgtggtatttttgttttaattattgctacaggtaaacttctctgaagacttgattttagtccctgggaaggaagcttctaagatgatcatccggagggccaaccaagctggtgtgataagagcggataaggacaatgttagaacggtggattccttcttgatgcatccttctagaagggtgtttaagaggttgtttatcgatgaaggactaatgctgcatacaggttgtgtaaatttcctactgctgctatctcaatgtgacgtcgcatatgtgtatggggacacaaagcaaattccgttcatttgcagagtcgcgaactttccgtatccagcgcattttgcaaaactcgtcgctgatgagaaggaagtcagaagagttacgctcaggtaaagcaactgtgttttaatcaatttcttgtcaggatatatggattataacttaatttttgagaaatctgtagtatttggcgtgaaatgagtttgctttttggtttctcccgtgttataggtgcccggctgatgttacgtatttccttaacaagaagtatgacggggcggtgatgtgtaccagcgcggtagagagatccgtgaaggcagaagtggtgagaggaaagggtgcattgaacccaataaccttaccgttggagggtaaaattttgaccttcacacaagctgacaagttcgagttactggagaagggttacaaggtaaagtttccaactttcctttaccatatcaaactaaagttcgaaactttttatttgatcaacttcaaggccacccgatctttctattcctgattaatttgtgatgaatccatattgacttttgatggttacgcaggatgtgaacactgtgcacgaggtgcaaggggagacgtacgagaagactgctattgtgcgcttgacatcaactccgttagagatcatatcgagtgcgtcacctcatgttttggtggcgctgacaagacacacaacgtgttgtaaatattacaccgttgtgttggacccgatggtgaatgtgatttcagaaatggagaagttgtccaatttccttcttgacatgtatagagttgaagcaggtctgtctttcctatttcatatgtttaatcctaggaatttgatcaattgattgtatgtatgtcgatcccaagactttcttgttcacttatatcttaactctctctttgctgtttcttgcaggtgtccaatagcaattacaaatcgatgcagtattcaggggacagaacttgtttgttcagacgcccaagtcaggagattggcgagatatgcaattttactatgacgctcttcttcccggaaacagtactattctcaatgaatttgatgctgttacgatgaatttgagggatatttccttaaacgtcaaagattgcagaatcgacttctccaaatccgtgcaacttcctaaagaacaacctattttcctcaagcctaaaataagaactgcggcagaaatgccgagaactgcaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtttgctggaaaatttggttgcaatgatcaaaagaaacatgaatgcgccggatttgacagggacaattgacattgaggatactgcatctctggtggttgaaaagttttgggattcgtatgttgacaaggaatttagtggaacgaacgaaatgaccatgacaagggagagcttctccaggtaaggacttctcatgaatattagtggcagattagtgttgttaaagtctttggttagataatcgatgcctcctaattgtccatgttttactggttttctacaattaaaggtggctttcgaaacaagagtcatctacagttggtcagttagcggactttaactttgtggatttgccggcagtagatgagtacaagcatatgatcaagagtcaaccaaagcaaaagttagacttgagtattcaagacgaatatcctgcattgcagacgatagtctaccattcgaaaaagatcaatgcgattttcggtccaatgttttcagaacttacgaggatgttactcgaaaggattgactcttcgaagtttctgttctacaccagaaagacacctgcacaaatagaggacttcttttctgacctagactcaacccaggcgatggaaattctggaactcgacatttcgaagtacgataagtcacaaaacgagttccattgtgctgtagagtacaagatctgggaaaagttaggaattgatgagtggctagctgaggtctggaaacaaggtgagttcctaagttccatttttttgtaatccttcaatgttattttaacttttcagatcaacatcaaaattaggttcaattttcatcaaccaaataatatttttcatgtatatataggtcacagaaaaacgaccttgaaagattatacggccggaatcaaaacatgtctttggtatcaaaggaaaagtggtgatgtgacaacctttattggtaataccatcatcattgccgcatgtttgagctcaatgatccccatggacaaagtgataaaggcagctttttgtggagacgatagcctgatttacattcctaaaggtttagacttgcctgatattcaggcgggcgcgaacctcatgtggaacttcgaggccaaactcttcaggaagaagtatggttacttctgtggtcgttatgttattcaccatgatagaggagccattgtgtattacgatccgcttaaactaatatctaagttaggttgtaaacatattagagatgttgttcacttagaagagttacgcgagtctttgtgtgatgtagctagtaacttaaataattgtgcgtatttttcacagttagatgaggccgttgccgaggttcataagaccgcggtaggcggttcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagccgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggtaaggatttttatgatatagtatgcttatgtattttgtactgaaagcatatcctgcttcattgggatattactgaaagcatttaactacatgtaaactcacttgatgatcaataaacttgattttgcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtaagccatcttcctgcttatttttataatgaacatagaaataggaagttgtgcagagaaactaattaacctgactcaaaatctaccctcataattgttgtttgatattggtcttgtattttgcaggtgtggttgacgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaacgtcttacaacattacaaacccgaatcagtaccagtatttcgataagctagcaaacaagaaatggggaagcaaatggccgccctgtgtggctttctcctcgtggcgttgctctggctcacgcccgacgtcgcgtcaggtgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcctaagcttactagagcgtggtgcgcacgatagcgcatagtgtttttctctccacttgaatcgaagagatagacttacggtgtaaatccgtaggggtggcgtaaaccaaattacgcaatgttttgggttccatttaaatcgaaaccccttatttcctggatcacctgttaacgcacgtttgacgtgtattacagtgggaataagtaaaagtgagaggttcgaatcctccctaaccccgggtaggggcccagcggccgctctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 12 - DNA, 10326 bp, Unknown
  Nucleotide sequence of T-DNA region of pICH95397
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctagcttggaattggtaccacgcgtttcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaagttttagttttattgcaacaacaacaacaaattacaataacaacaaacaaaatacaaacaacaacaacatggcacaatttcaacaaacaattgacatgcaaactctccaagccgctgcgggacgcaacagcttggtgaatgatttggcatctcgtcgcgtttacgataatgcagtcgaggagctgaatgctcgttccagacgtcccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcaggtccacttctccaaggcagtgtctacggaacagaccctgattgcaacaaacgcatatccggagttcgagatttcctttactcatacgcaatccgctgtgcactccttggccggaggccttcggtcacttgagttggagtatctcatgatgcaagttccgttcggttctctgacgtacgacatcggcggtaacttttccgcgcaccttttcaaagggcgcgattacgttcactgctgcatgcctaatctggatgtacgtgacattgctcgccatgaaggacacaaggaagctatttacagttatgtgaatcgtttgaaaaggcagcagcgtcctgtgcctgaataccagagggcagctttcaacaactacgctgagaacccgcacttcgtccattgcgacaaacctttccaacagtgtgaattgacgacagcgaatggcactgacacctacgctgtagctctccatagcatttatgatatccctgttgaggagttcggttctgcgctactcaggaagaatgtgaaaacttgtttcgcggcctttcatttccatgagaatatgcttctagattgtgatacagtcacactcgatgagattggagctacgttccagaaatcaggtaacattccttagttacctttcttttctttttccatcataagtttatagattgtacatgctttgagatttttctttgcaaacaatctcaggtgataacctgagcttcttcttccataatgagagcactctcaattacacccacagcttcagcaacatcatcaagtacgtgtgcaagacgttcttccctgctagtcaacgcttcgtgtaccacaaggagttcctggtcactagagtcaacacttggtactgcaagttcacgagagtggatacgttcactctgttccgtggtgtgtaccacaacaatgtggattgcgaagagttttacaaggctatggacgatgcgtggcactacaaaaagacgttagcaatgcttaatgccgagaggaccatcttcaaggataacgctgcgttaaacttctggttcccgaaggtgctcttgaaattggaagtcttcttttgttgtctaaacctatcaatttctttgcggaaatttatttgaagctgtagagttaaaattgagtcttttaaacttttgtaggtgagagacatggttatcgtccctctctttgacgcttctatcacaactggtaggatgtctaggagagaggttatggtgaacaaggacttcgtctacacggtcctaaatcacatcaagacctatcaagctaaggcactgacgtacgcaaacgtgctgagcttcgtggagtctattaggtctagagtgataattaacggtgtcactgccaggtaagttgttacttatgattgttttcctctctgctacatgtattttgttgttcatttctgtaagatataagaattgagttttcctctgatgatattattaggtctgaatgggacacagacaaggcaattctaggtccattagcaatgacattcttcctgatcacgaagctgggtcatgtgcaagatgaaataatcctgaaaaagttccagaagttcgacagaaccaccaatgagctgatttggacaagtctctgcgatgccctgatgggggttattccctcggtcaaggagacgcttgtgcgcggtggttttgtgaaagtagcagaacaagccttagagatcaaggttagtatcatatgaagaaatacctagtttcagttgatgaatgctattttctgacctcagttgttctcttttgagaattatttcttttctaatttgcctgatttttctattaattcattaggttcccgagctatactgtaccttcgccgaccgattggtactacagtacaagaaggcggaggagttccaatcgtgtgatctttccaaacctctagaagagtcagagaagtactacaacgcattatccgagctatcagtgcttgagaatctcgactcttttgacttagaggcgtttaagactttatgtcagcagaagaatgtggacccggatatggcagcaaaggtaaatcctggtccacacttttacgataaaaacacaagattttaaactatgaactgatcaataatcattcctaaaagaccacacttttgttttgtttctaaagtaatttttactgttataacaggtggtcgtagcaatcatgaagtcagaattgacgttgcctttcaagaaacctacagaagaggaaatctcggagtcgctaaaaccaggagaggggtcgtgtgcagagcataaggaagtgttgagcttacaaaatgatgctccgttcccgtgtgtgaaaaatctagttgaaggttccgtgccggcgtatggaatgtgtcctaagggtggtggtttcgacaaattggatgtggacattgctgatttccatctcaagagtgtagatgcagttaaaaagggaactatgatgtctgcggtgtacacagggtctatcaaagttcaacaaatgaagaactacatagattacttaagtgcgtcgctggcagctacagtctcaaacctctgcaaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcttagagatgttcacggcgttgacccagagtcacaggagaaatctggagtgtgggatgttaggagaggacgttggttacttaaacctaatgcgaaaagtcacgcgtggggtgtggcagaagacgccaaccacaagttggttattgtgttactcaactgggatgacggaaagccggtttgtgatgagacatggttcagggtggcggtgtcaagcgattccttgatatattcggatatgggaaaacttaagacgctcacgtcttgcagtccaaatggtgagccaccggagcctaacgccaaagtaattttggtcgatggtgttcccggttgtggaaaaacgaaggagattatcgaaaaggtaagttctgcatttggttatgctccttgcattttaggtgttcgtcgctcttccatttccatgaatagctaagattttttttctctgcattcattcttcttgcctcagttctaactgtttgtggtatttttgttttaattattgctacaggtaaacttctctgaagacttgattttagtccctgggaaggaagcttctaagatgatcatccggagggccaaccaagctggtgtgataagagcggataaggacaatgttagaacggtggattccttcttgatgcatccttctagaagggtgtttaagaggttgtttatcgatgaaggactaatgctgcatacaggttgtgtaaatttcctactgctgctatctcaatgtgacgtcgcatatgtgtatggggacacaaagcaaattccgttcatttgcagagtcgcgaactttccgtatccagcgcattttgcaaaactcgtcgctgatgagaaggaagtcagaagagttacgctcaggtaaagcaactgtgttttaatcaatttcttgtcaggatatatggattataacttaatttttgagaaatctgtagtatttggcgtgaaatgagtttgctttttggtttctcccgtgttataggtgcccggctgatgttacgtatttccttaacaagaagtatgacggggcggtgatgtgtaccagcgcggtagagagatccgtgaaggcagaagtggtgagaggaaagggtgcattgaacccaataaccttaccgttggagggtaaaattttgaccttcacacaagctgacaagttcgagttactggagaagggttacaaggtaaagtttccaactttcctttaccatatcaaactaaagttcgaaactttttatttgatcaacttcaaggccacccgatctttctattcctgattaatttgtgatgaatccatattgacttttgatggttacgcaggatgtgaacactgtgcacgaggtgcaaggggagacgtacgagaagactgctattgtgcgcttgacatcaactccgttagagatcatatcgagtgcgtcacctcatgttttggtggcgctgacaagacacacaacgtgttgtaaatattacaccgttgtgttggacccgatggtgaatgtgatttcagaaatggagaagttgtccaatttccttcttgacatgtatagagttgaagcaggtctgtctttcctatttcatatgtttaatcctaggaatttgatcaattgattgtatgtatgtcgatcccaagactttcttgttcacttatatcttaactctctctttgctgtttcttgcaggtgtccaatagcaattacaaatcgatgcagtattcaggggacagaacttgtttgttcagacgcccaagtcaggagattggcgagatatgcaattttactatgacgctcttcttcccggaaacagtactattctcaatgaatttgatgctgttacgatgaatttgagggatatttccttaaacgtcaaagattgcagaatcgacttctccaaatccgtgcaacttcctaaagaacaacctattttcctcaagcctaaaataagaactgcggcagaaatgccgagaactgcaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtttgctggaaaatttggttgcaatgatcaaaagaaacatgaatgcgccggatttgacagggacaattgacattgaggatactgcatctctggtggttgaaaagttttgggattcgtatgttgacaaggaatttagtggaacgaacgaaatgaccatgacaagggagagcttctccaggtaaggacttctcatgaatattagtggcagattagtgttgttaaagtctttggttagataatcgatgcctcctaattgtccatgttttactggttttctacaattaaaggtggctttcgaaacaagagtcatctacagttggtcagttagcggactttaactttgtggatttgccggcagtagatgagtacaagcatatgatcaagagtcaaccaaagcaaaagttagacttgagtattcaagacgaatatcctgcattgcagacgatagtctaccattcgaaaaagatcaatgcgattttcggtccaatgttttcagaacttacgaggatgttactcgaaaggattgactcttcgaagtttctgttctacaccagaaagacacctgcacaaatagaggacttcttttctgacctagactcaacccaggcgatggaaattctggaactcgacatttcgaagtacgataagtcacaaaacgagttccattgtgctgtagagtacaagatctgggaaaagttaggaattgatgagtggctagctgaggtctggaaacaaggtgagttcctaagttccatttttttgtaatccttcaatgttattttaacttttcagatcaacatcaaaattaggttcaattttcatcaaccaaataatatttttcatgtatatataggtcacagaaaaacgaccttgaaagattatacggccggaatcaaaacatgtctttggtatcaaaggaaaagtggtgatgtgacaacctttattggtaataccatcatcattgccgcatgtttgagctcaatgatccccatggacaaagtgataaaggcagctttttgtggagacgatagcctgatttacattcctaaaggtttagacttgcctgatattcaggcgggcgcgaacctcatgtggaacttcgaggccaaactcttcaggaagaagtatggttacttctgtggtcgttatgttattcaccatgatagaggagccattgtgtattacgatccgcttaaactaatatctaagttaggttgtaaacatattagagatgttgttcacttagaagagttacgcgagtctttgtgtgatgtagctagtaacttaaataattgtgcgtatttttcacagttagatgaggccgttgccgaggttcataagaccgcggtaggcggttcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagccgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggtaaggatttttatgatatagtatgcttatgtattttgtactgaaagcatatcctgcttcattgggatattactgaaagcatttaactacatgtaaactcacttgatgatcaataaacttgattttgcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtaagccatcttcctgcttatttttataatgaacatagaaataggaagttgtgcagagaaactaattaacctgactcaaaatctaccctcataattgttgtttgatattggtcttgtattttgcaggtgtggttgacgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaacgtcttacaacattacaaacccgaatcagtaccagtatttcgataagctagcaaacaagaaatggggaagcaaatggccgccctgtgtggctttctcctcgtggcgttgctctggctcacgcccgacgtcgcgtcaggtgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcctaagcttactagagcgtggtgcgcacgatagcgcatagtgtttttctctccacttgaatcgaagagatagacttacggtgtaaatccgtaggggtggcgtaaaccaaattacgcaatgttttgggttccatttaaatcgaaaccccttatttcctggatcacctgttaacgcacgtttgacgtgtattacagtgggaataagtaaaagtgagaggttcgaatcctccctaaccccgggtaggggcccagcggccgctctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 13 - DNA, 17364 bp, Unknown
  Nucleotide sequence of T-DNA region of pNMD40523
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccactctgtgtgcctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatacgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattgtaattgtaaatagtaattgtaatgttgtttgttgtttgttgttgttggtaattgttgtaaaaatacagtatgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctgcaagaattcaagcttggaggtgttcatctgttggacaagctaataatatctatttgttgtctgaatcacaattttcccatgataaattaaagccaaaattcggcttctcacaacctgatagaatctaattttatgacttttatcaaaaaaaagaagaagatagaatctaattttatgacatttaatggtagccattccttaaaaattcccgattcaactaatccttggagttcattttctcataggtggatttcatgctggagttactgtaaatagggttggatttctacattcttgcgagagtgatctgcattccagccccctctgaagcacaggagtcagctgcatttcaaatgattacccaattacagactatggcagaccgcacccattagatttaatgtggcaaagaaacatcaactccaatatttttgaatcctattaatggctatcataccatagtactgtattagtactgtccagaagcaaaaattgttgttttagaatattacttggttgcaaccgtatcagtgctttgaacatttatcaaggccaaatacataatggtgccttgaaatcaactctagtttattgaaatgatgtctattgagcacctatatgtgttgccagtattgcccatgcttaacttagcattctgttaacaagtgttatgctggatgttcgaatcgaacatggagttttcatgttgcttagaactatatgctttcagtgttagcagtatcggaaggtaaatacagtttaaacaccaaaagtagagagaaataaggaggaatttaatctttggatttgattcgtctttcttgtgggaatggtgaactcgttaaccagctaggtgaaatgaactcaatacttagttcaaaattgtttaagttccatttttggttaagagaattgagttgtgctctctagcttttgaagatgatttaggttcaaggactataattttacacgatattagagctagatccattgataggccgcgtttcttaatgtttttggccttgtaattctgttttcacattgattgactgaatgagttgtgctctttctatatgatctcggataattctttttctcatgagctaacttaagttaggatatgagctaactgctctccaacaggcaacaacccatagaggaaccaatcagttttttctttttgtcctcctcttccttgcacgagtggacttagtaaatagaaaggaaaataacaaaaatgtataagaaacaagtcaatctcttgggtcccttaataatcaaagaaaaccaatgaatgaactccaagtgcacaaaactaagataaggcaaaggaaaataatttgtagtaagaatgctatatattgataatgttggtaataatattatctagaagcaatccacagaaggggtgccacgtgtcaacttgtaaatcacaaattgcaagtcatcactaatattattatcaagggaccatgcaaagtgaaaataaataattcatactaagtagtgagagcaatacttctatttttacaacaattaccaacaacaacaaacaacaaacaacattacaattacatttacaattaccatggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttgtaggcttggtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtagctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtgttccattcacaaatgaacttaaactagcacaaagtcagatattttaagatcgcaccatttcgctactagaattcgagctcagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggtacctgggcccctacccggggttagggaggattcgaacctctcacttttacttattcccactgtaatacacgtcaaacgtgcgttaacaggtgatccaggaaataaggggtttcgatttaaatggaacccaaaacattgcgtaatttggtttacgccacccctacggatttacaccgtaagtctatctcttcgattcaagtggagagaaaaacactatgcgctatcgtgcgcaccacgctctagtaagcttaggcagtagggcagaaagtgaccctgtagttggagctgccggggcaggtgacggtggttggcttgtccaggacataactgaacgcgtccgggcaaagcctcttgaagaagcgcgagtactccgtcggcccgcacttccccgtggtgcagcagtactcgctcgtctggaacacggtgcacgcatcgttgcaaccaccccccggcgccttcagcttcgccgggcactgccccacgatgtcggcggcgcaccgcaccccgcggcagccgcgcgtggtcgggctgaagtccatcggcacgttgaagcctttgatgttggagatgtcgatgtagtccttgccgtactggttgagcgagaactccgccagcgtggtgggcggccggccgaagcgcttgcaccggaggaggccgccgcagtcgccggtcttgcagatgccgctgccgctgtcgtcgaaatagcagtcggtgcgggcccagattttgccaccgttggtgccgggttctacgttgatggtccaggactctcccgagttgagctggcggccgccggcgtccagggcggcgtcgcctttggaggcggccgcccacacggtgtaggagcagcggttgacgatctcgaaggtggcacctgacgcgacgtcgggcgtgagccagagcaacgccacgaggagaaagccacacagggcggccatttgcttccccattgtttcttgtttcttatcgaaatactggtactgattcgggtttgtaatgttgtaagacgttttcttctttcaaattaaacgaatcaggaccggcgtacttctccggtctgtacttatacttacttactacacccttttcttcaaccttctttttccttcttttaaagttgtcaaccgctttaaatgctgcaaccgaatcgacgaattctaacctcgttagagccttcggcaagatctcttccttcttcgaaagattgaggaaatcactcaccttaggttcgtacgagactatcgacattatcaaacaaagaacaaatctctaaacaatctcttatctgacaaatacttaattatactacaaaaagcaaacgaaccgcctaccgcggtcttatgaacctcggcaacggcctcatctaactgtgaaaaatacgcacaattatttaagttactagctacatcacacaaagactcgcgtaactcttctaagtgaacaacatctctaatatgtttacaacctaacttagatattagtttaagcggatcgtaatacacaatggctcctctatcatggtgaataacataacgaccacagaagtaaccatacttcttcctgaagagtttggcctcgaagttccacatgaggttcgcgcccgcctgaatatcaggcaagtctaaacctttaggaatgtaaatcaggctatcgtcgccacaaaaagctgcctttatcactttgtccatggggatcattgagctcaaacatgcggcaatgatgatggtattaccaataaaggttgtcacatcaccacttttcctttgataccaaagacatgttttgattccggccgtataatctttcaaggtcgtttttctgtgacctatatatacatgaaaaatattatttggttgatgaaaattgaacctaattttgatgttgatctgaaaagttaaaataacattgaaggattacaaaaaaatggaacttaggaactcaccttgtttccagacctcagctagccactcatcaattcctaacttttcccagatcttgtactctacagcacaatggaactcgttttgtgacttatcgtacttcgaaatgtcgagttccagaatttccatcgcctgggttgagtctaggtcagaaaagaagtcctctatttgtgcaggtgtctttctggtgtagaacagaaacttcgaagagtcaatcctttcgagtaacatcctcgtaagttctgaaaacattggaccgaaaatcgcattgatctttttcgaatggtagactatcgtctgcaatgcaggatattcgtcttgaatactcaagtctaacttttgctttggttgactcttgatcatatgcttgtactcatctactgccggcaaatccacaaagttaaagtccgctaactgaccaactgtagatgactcttgtttcgaaagccacctttaattgtagaaaaccagtaaaacatggacaattaggaggcatcgattatctaaccaaagactttaacaacactaatctgccactaatattcatgagaagtccttacctggagaagctctcccttgtcatggtcatttcgttcgttccactaaattccttgtcaacatacgaatcccaaaacttttcaaccaccagagatgcagtatcctcaatgtcaattgtccctgtcaaatccggcgcattcatgtttcttttgatcattgcaaccaaattttccagcaaacctggtttgtataattttggttcagttaattaggaaaattacataatggctccaaaaaaaaattatcgaccttttaccaaaaaaaataaaatttatcgaccttgacaggaaaaagttacaaatcaaaagaaagaatatcgtctggcatccaatattttacctgcagttctcggcatttctgccgcagttcttattttaggcttgaggaaaataggttgttctttaggaagttgcacggatttggagaagtcgattctgcaatctttgacgtttaaggaaatatccctcaaattcatcgtaacagcatcaaattcattgagaatagtactgtttccgggaagaagagcgtcatagtaaaattgcatatctcgccaatctcctgacttgggcgtctgaacaaacaagttctgtcccctgaatactgcatcgatttgtaattgctattggacacctgcaagaaacagcaaagagagagttaagatataagtgaacaagaaagtcttgggatcgacatacatacaatcaattgatcaaattcctaggattaaacatatgaaataggaaagacagacctgcttcaactctatacatgtcaagaaggaaattggacaacttctccatttctgaaatcacattcaccatcgggtccaacacaacggtgtaatatttacaacacgttgtgtgtcttgtcagcgccaccaaaacatgaggtgacgcactcgatatgatctctaacggagttgatgtcaagcgcacaatagcagttttctcgtatgtctccccttgcacctcgtgcacagtgttcacatcctgcgtaaccatcaaaagtcaatatggattcatcacaaattaatcaggaatagaaagatcgggtggccttgaagttgatcaaataaaaagtttcgaactttagtttgatatggtaaaggaaagttggaaactttaccttgtaacccttctccagtaactcgaacttgtcagcttgtgtgaaggtcaaaattttaccctccaacggtaaggttattgggttcaatgcaccctttcctctcaccacttctgccttcacggatctctctaccgcgctggtacacatcaccgccccgtcatacttcttgttaaggaaatacgtaacatcagccgggcacctataacacgggagaaaccaaaaagcaaactcatttcacgccaaatactacagatttctcaaaaattaagttataatccatatatcctgacaagaaattgattaaaacacagttgctttacctgagcgtaactcttctgacttccttctcatcagcgacgagttttgcaaaatgcgctggatacggaaagttcgcgactctgcaaatgaacggaatttgctttgtgtccccatacacatatgcgacgtcacattgagatagcagcagtaggaaatttacacaacctgtatgcagcattagtccttcatcgataaacaacctcttaaacacccttctagaaggatgcatcaagaaggaatccaccgttctaacattgtccttatccgctcttatcacaccagcttggttggccctccggatgatcatcttagaagcttccttcccagggactaagatcaagtcctcagagaagtttacctgtagcaataattaaaacaaaaataccacaaacagttagaactgaggcaagaagaatgaatgcagagaaaaaaaatcttagctattcatggaaatggaagagcgacgaacacctaaaatgcaaggagcataaccaaatgcagaacttaccttttcgataatctccttcgtttttccacaaccgggaacaccatcgaccaaaattactttggcgttaggctccggtggctcaccatttggactgcaagacgtgagcgtcttaagttttcccatatccgaatatatcaaggaatcgcttgacaccgccaccctgaaccatgtctcatcacaaaccggctttccgtcatcccagttgagtaacacaataaccaacttgtggttggcatcttctgccacaccccacgcgtgacttttcgcattaggtttaagtaaccaacgtcctctcctaacatcccacactccagatttctcctgtgactctgggtcaacgccgtgaacatctctaagcacctaaaacatagaacaaacatttgtctcatcacaaatatgcatcaaggaacactcagaagttagtcatatacccaaattcttgaaactagagaaacaaaaaaagagggatcattgcggaaaccttttgacctcttaccttgcagaggtttgagactgtagctgccagcgacgcacttaagtaatctatgtagttcttcatttgttgaactttgatagaccctgtgtacaccgcagacatcatagttccctttttaactgcatctacactcttgagatggaaatcagcaatgtccacatccaatttgtcgaaaccaccacccttaggacacattccatacgccggcacggaaccttcaactagatttttcacacacgggaacggagcatcattttgtaagctcaacacttccttatgctctgcacacgacccctctcctggttttagcgactccgagatttcctcttctgtaggtttcttgaaaggcaacgtcaattctgacttcatgattgctacgaccacctgttataacagtaaaaattactttagaaacaaaacaaaagtgtggtcttttaggaatgattattgatcagttcatagtttaaaatcttgtgtttttatcgtaaaagtgtggaccaggatttacctttgctgccatatccgggtccacattcttctgctgacataaagtcttaaacgcctctaagtcaaaagagtcgagattctcaagcactgatagctcggataatgcgttgtagtacttctctgactcttctagaggtttggaaagatcacacgattggaactcctccgccttcttgtactgtagtaccaatcggtcggcgaaggtacagtatagctcgggaacctttatctctaaggcttgttctgctactttcacaaaaccaccgcgcacaagcgtctccttgaccgagggaataacccccatcagggcatcgcagagacttgtccaaatcagctcattggtggttctgtcgaacttctggaactttttcaggattatttcatcctgcacatgacccaactttgttataaggaaaaatgtcattgctaatggacctagaattgccttgtctgtgtcccattcagacctggcagtgacaccgttaattatcactctagacctaatagactccacaaaggacagaacatttgcgtaagttaaagccttagcttgatacgtttttatgtgatttaggaccgtataaacgaaatccttgttcaccataatctctctcctagacatcctaccagttgtgatagaagcgtcaaagagagggacgataaccatgtctctcactttcgggaaccaaaagtttaacgcagcgttatccttgaagatggtcctctcggcattaagcattgctaacgtctttttgtagtgccacgcatcgtccatagccttgtaaaactcttcgcaatccacattattatggtacacaccacggaaaagagtaaaagtatccactctcgtaaacttacagtaccaagtgttgactctagtaactaaaaactccttatgatacacaaaccgttgactagcaggaaagaacgttttacacacatacttaattatattactaaaactgtgggtgtaattgagagtgctctcattatgaaagaaaaaacttaaattatcaccggacttctgaaaagtagctccaatctcatcgagtgtgactgtatcacaatctagaagcatattctcatggaaatgaaaggccgcgaaacaagttttcacattcttcctgagtagcgcagaaccgaactcctcaacagggatatcataaatgctatggagagctacagcgtaggtgtcagtgccatacgctgtcgtcaattcacactgttggaaaggtttgtcgcaatggacgaagtgcgggttctcagcgtagttgttgaaagctgccctctggtattcaggcacaggacgctgctgccttttcaaacgattcacataactgtaaatagcttccttgtgtccttcatggcgagcaatgtcacgtacatccagattaggcatgcagcagtgaacgtaatcgcgccctttgaaaaggtgcgcggaaaagttaccgccgatgtcgtacgtcagagaaccgaacggaacttgcatcatgagatactccaactcaagtgaccgaaggcctccggccaaggagtgcacagcggattgcgtatgagtaaaggaaatctcgaactccggatatgcgtttgttgcaatcagtgtctgttccgtagacactgccttggagaaatgaaccttgggacgtctggaacgagcattcagctcctcgactgcattatcgtaaacgcgacgagatgccaaatcattcaccaagctgttgcgtcccgcagcggcttggagagtttgcatgtcaattgtttgttgaaattgtgccatgttgttgttgtttgtattttgtttgttgttattgtaatttgttgttgttgttgcaataaaactaaaacagtactccaaatgaaatgaacttccttatatagatgttcagctatgcggggatgcgatcctggggtaggagagcacgtacggggccccgctcgtttgtggctctccgtgcggacatcccgtgcggacagtaccagaaagtgctctgtctctgctctatacggctctatacgcgtacctcttgaacggtgcgtggagaggagtggtgtgtcaatttccgccccgccctcgtgcggttccgcatgcatccaatctccttacgaattcccatggggagactagaattcgagctcggagaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgctattggatgcatgcggaaccgcacgagggcggggcggaaattgacacaccactcctctccacgcaccgttcaagaggtacgcgtatagagccgtatagagcagagacagagcactttctggtactgtccgcacgggatgtccgcacggagagccacaaacgagcggggccccgtacgtgctctcctaccccaggatcgcatccccgcatagctgaacatctatataaggaagttcatttcatttggagtactaggacgacctcgagtataagagctcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagctgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtgtggttgacgaattcgtcgattcggttgcagcattcaaggcggtcgacaacttcaggaagaggaagaagaaggttgaagagagggatgtagtcagcaagtacaagtacagaccggagaagtacgccggtcctgattcgttcaacctgaaggaggaaaacgtcctccagcactacaagcccgaataagcttggtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtagtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctcagagaggatgcacatgtgaccgagggacacgaagtggtttaaactatcagtgtttgacaggatatattggcgggtaaacctaagagaaaagagcgtttattagaataatcggatatttaaaagggcgtgaaaaggtttatccgttcgtccatttgtatgtgccaacca

SEQ ID NO: 14 - DNA, 17364 bp, Unknown
  Nucleotide sequence of T-DNA region of pNMD38061
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccactctgtgtgcctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatacgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattgtaattgtaaatagtaattgtaatgttgtttgttgtttgttgttgttggtaattgttgtaaaaatacagtatgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctgcaagaattcaagcttggaggtgttcatctgttggacaagctaataatatctatttgttgtctgaatcacaattttcccatgataaattaaagccaaaattcggcttctcacaacctgatagaatctaattttatgacttttatcaaaaaaaagaagaagatagaatctaattttatgacatttaatggtagccattccttaaaaattcccgattcaactaatccttggagttcattttctcataggtggatttcatgctggagttactgtaaatagggttggatttctacattcttgcgagagtgatctgcattccagccccctctgaagcacaggagtcagctgcatttcaaatgattacccaattacagactatggcagaccgcacccattagatttaatgtggcaaagaaacatcaactccaatatttttgaatcctattaatggctatcataccatagtactgtattagtactgtccagaagcaaaaattgttgttttagaatattacttggttgcaaccgtatcagtgctttgaacatttatcaaggccaaatacataatggtgccttgaaatcaactctagtttattgaaatgatgtctattgagcacctatatgtgttgccagtattgcccatgcttaacttagcattctgttaacaagtgttatgctggatgttcgaatcgaacatggagttttcatgttgcttagaactatatgctttcagtgttagcagtatcggaaggtaaatacagtttaaacaccaaaagtagagagaaataaggaggaatttaatctttggatttgattcgtctttcttgtgggaatggtgaactcgttaaccagctaggtgaaatgaactcaatacttagttcaaaattgtttaagttccatttttggttaagagaattgagttgtgctctctagcttttgaagatgatttaggttcaaggactataattttacacgatattagagctagatccattgataggccgcgtttcttaatgtttttggccttgtaattctgttttcacattgattgactgaatgagttgtgctctttctatatgatctcggataattctttttctcatgagctaacttaagttaggatatgagctaactgctctccaacaggcaacaacccatagaggaaccaatcagttttttctttttgtcctcctcttccttgcacgagtggacttagtaaatagaaaggaaaataacaaaaatgtataagaaacaagtcaatctcttgggtcccttaataatcaaagaaaaccaatgaatgaactccaagtgcacaaaactaagataaggcaaaggaaaataatttgtagtaagaatgctatatattgataatgttggtaataatattatctagaagcaatccacagaaggggtgccacgtgtcaacttgtaaatcacaaattgcaagtcatcactaatattattatcaagggaccatgcaaagtgaaaataaataattcatactaagtagtgagagcaatacttctatttttacaacaattaccaacaacaacaaacaacaaacaacattacaattacatttacaattaccatggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttgtaggcttggtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtagctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtgttccattcacaaatgaacttaaactagcacaaagtcagatattttaagatcgcaccatttcgctactagaattcgagctcagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggtacctgggcccctacccggggttagggaggattcgaacctctcacttttacttattcccactgtaatacacgtcaaacgtgcgttaacaggtgatccaggaaataaggggtttcgatttaaatggaacccaaaacattgcgtaatttggtttacgccacccctacggatttacaccgtaagtctatctcttcgattcaagtggagagaaaaacactatgcgctatcgtgcgcaccacgctctagtaagcttaggcagtagggcagaaagtgaccctgtagttggagctgccggggcaggtgacggtggttggcttgtccaggacataactgaacgcgtccgggcaaagcctcttgaagaagcgcgagtactccgtcggcccgcacttccccgtggtgcagcagtactcgctcgtctggaacacggtgcacgcatcgttgcaaccaccccccggcgccttcagcttcgccgggcactgccccacgatgtcggcggcgcaccgcaccccgcggcagccgcgcgtggtcgggctgaagtccatcggcacgttgaagcctttgatgttggagatgtcgatgtagtccttgccgtactggttgagcgagaactccgccagcgtggtgggcggccggccgaagcgcttgcactggaggaggccgccgcagtcgccggtccggcagatgccgcggccgctgtcgtcgaaatagcagtcggtgcgggcccagattttgccacccttggtgccgggttctacgttgatggtccaggactctcccgagttgagctggcggccgccggcgtccagggcggcgtcgcctttggaggcggccgcccacacggtgtaggagcagcggttgacgatctcgaaggtggcacctgacgcgacgtcgggcgtgagccagagcaacgccacgaggagaaagccacacagggcggccatttgcttccccattgtttcttgtttcttatcgaaatactggtactgattcgggtttgtaatgttgtaagacgttttcttctttcaaattaaacgaatcaggaccggcgtacttctccggtctgtacttatacttacttactacacccttttcttcaaccttctttttccttcttttaaagttgtcaaccgctttaaatgctgcaaccgaatcgacgaattctaacctcgttagagccttcggcaagatctcttccttcttcgaaagattgaggaaatcactcaccttaggttcgtacgagactatcgacattatcaaacaaagaacaaatctctaaacaatctcttatctgacaaatacttaattatactacaaaaagcaaacgaaccgcctaccgcggtcttatgaacctcggcaacggcctcatctaactgtgaaaaatacgcacaattatttaagttactagctacatcacacaaagactcgcgtaactcttctaagtgaacaacatctctaatatgtttacaacctaacttagatattagtttaagcggatcgtaatacacaatggctcctctatcatggtgaataacataacgaccacagaagtaaccatacttcttcctgaagagtttggcctcgaagttccacatgaggttcgcgcccgcctgaatatcaggcaagtctaaacctttaggaatgtaaatcaggctatcgtcgccacaaaaagctgcctttatcactttgtccatggggatcattgagctcaaacatgcggcaatgatgatggtattaccaataaaggttgtcacatcaccacttttcctttgataccaaagacatgttttgattccggccgtataatctttcaaggtcgtttttctgtgacctatatatacatgaaaaatattatttggttgatgaaaattgaacctaattttgatgttgatctgaaaagttaaaataacattgaaggattacaaaaaaatggaacttaggaactcaccttgtttccagacctcagctagccactcatcaattcctaacttttcccagatcttgtactctacagcacaatggaactcgttttgtgacttatcgtacttcgaaatgtcgagttccagaatttccatcgcctgggttgagtctaggtcagaaaagaagtcctctatttgtgcaggtgtctttctggtgtagaacagaaacttcgaagagtcaatcctttcgagtaacatcctcgtaagttctgaaaacattggaccgaaaatcgcattgatctttttcgaatggtagactatcgtctgcaatgcaggatattcgtcttgaatactcaagtctaacttttgctttggttgactcttgatcatatgcttgtactcatctactgccggcaaatccacaaagttaaagtccgctaactgaccaactgtagatgactcttgtttcgaaagccacctttaattgtagaaaaccagtaaaacatggacaattaggaggcatcgattatctaaccaaagactttaacaacactaatctgccactaatattcatgagaagtccttacctggagaagctctcccttgtcatggtcatttcgttcgttccactaaattccttgtcaacatacgaatcccaaaacttttcaaccaccagagatgcagtatcctcaatgtcaattgtccctgtcaaatccggcgcattcatgtttcttttgatcattgcaaccaaattttccagcaaacctggtttgtataattttggttcagttaattaggaaaattacataatggctccaaaaaaaaattatcgaccttttaccaaaaaaaataaaatttatcgaccttgacaggaaaaagttacaaatcaaaagaaagaatatcgtctggcatccaatattttacctgcagttctcggcatttctgccgcagttcttattttaggcttgaggaaaataggttgttctttaggaagttgcacggatttggagaagtcgattctgcaatctttgacgtttaaggaaatatccctcaaattcatcgtaacagcatcaaattcattgagaatagtactgtttccgggaagaagagcgtcatagtaaaattgcatatctcgccaatctcctgacttgggcgtctgaacaaacaagttctgtcccctgaatactgcatcgatttgtaattgctattggacacctgcaagaaacagcaaagagagagttaagatataagtgaacaagaaagtcttgggatcgacatacatacaatcaattgatcaaattcctaggattaaacatatgaaataggaaagacagacctgcttcaactctatacatgtcaagaaggaaattggacaacttctccatttctgaaatcacattcaccatcgggtccaacacaacggtgtaatatttacaacacgttgtgtgtcttgtcagcgccaccaaaacatgaggtgacgcactcgatatgatctctaacggagttgatgtcaagcgcacaatagcagttttctcgtatgtctccccttgcacctcgtgcacagtgttcacatcctgcgtaaccatcaaaagtcaatatggattcatcacaaattaatcaggaatagaaagatcgggtggccttgaagttgatcaaataaaaagtttcgaactttagtttgatatggtaaaggaaagttggaaactttaccttgtaacccttctccagtaactcgaacttgtcagcttgtgtgaaggtcaaaattttaccctccaacggtaaggttattgggttcaatgcaccctttcctctcaccacttctgccttcacggatctctctaccgcgctggtacacatcaccgccccgtcatacttcttgttaaggaaatacgtaacatcagccgggcacctataacacgggagaaaccaaaaagcaaactcatttcacgccaaatactacagatttctcaaaaattaagttataatccatatatcctgacaagaaattgattaaaacacagttgctttacctgagcgtaactcttctgacttccttctcatcagcgacgagttttgcaaaatgcgctggatacggaaagttcgcgactctgcaaatgaacggaatttgctttgtgtccccatacacatatgcgacgtcacattgagatagcagcagtaggaaatttacacaacctgtatgcagcattagtccttcatcgataaacaacctcttaaacacccttctagaaggatgcatcaagaaggaatccaccgttctaacattgtccttatccgctcttatcacaccagcttggttggccctccggatgatcatcttagaagcttccttcccagggactaagatcaagtcctcagagaagtttacctgtagcaataattaaaacaaaaataccacaaacagttagaactgaggcaagaagaatgaatgcagagaaaaaaaatcttagctattcatggaaatggaagagcgacgaacacctaaaatgcaaggagcataaccaaatgcagaacttaccttttcgataatctccttcgtttttccacaaccgggaacaccatcgaccaaaattactttggcgttaggctccggtggctcaccatttggactgcaagacgtgagcgtcttaagttttcccatatccgaatatatcaaggaatcgcttgacaccgccaccctgaaccatgtctcatcacaaaccggctttccgtcatcccagttgagtaacacaataaccaacttgtggttggcatcttctgccacaccccacgcgtgacttttcgcattaggtttaagtaaccaacgtcctctcctaacatcccacactccagatttctcctgtgactctgggtcaacgccgtgaacatctctaagcacctaaaacatagaacaaacatttgtctcatcacaaatatgcatcaaggaacactcagaagttagtcatatacccaaattcttgaaactagagaaacaaaaaaagagggatcattgcggaaaccttttgacctcttaccttgcagaggtttgagactgtagctgccagcgacgcacttaagtaatctatgtagttcttcatttgttgaactttgatagaccctgtgtacaccgcagacatcatagttccctttttaactgcatctacactcttgagatggaaatcagcaatgtccacatccaatttgtcgaaaccaccacccttaggacacattccatacgccggcacggaaccttcaactagatttttcacacacgggaacggagcatcattttgtaagctcaacacttccttatgctctgcacacgacccctctcctggttttagcgactccgagatttcctcttctgtaggtttcttgaaaggcaacgtcaattctgacttcatgattgctacgaccacctgttataacagtaaaaattactttagaaacaaaacaaaagtgtggtcttttaggaatgattattgatcagttcatagtttaaaatcttgtgtttttatcgtaaaagtgtggaccaggatttacctttgctgccatatccgggtccacattcttctgctgacataaagtcttaaacgcctctaagtcaaaagagtcgagattctcaagcactgatagctcggataatgcgttgtagtacttctctgactcttctagaggtttggaaagatcacacgattggaactcctccgccttcttgtactgtagtaccaatcggtcggcgaaggtacagtatagctcgggaacctttatctctaaggcttgttctgctactttcacaaaaccaccgcgcacaagcgtctccttgaccgagggaataacccccatcagggcatcgcagagacttgtccaaatcagctcattggtggttctgtcgaacttctggaactttttcaggattatttcatcctgcacatgacccaactttgttataaggaaaaatgtcattgctaatggacctagaattgccttgtctgtgtcccattcagacctggcagtgacaccgttaattatcactctagacctaatagactccacaaaggacagaacatttgcgtaagttaaagccttagcttgatacgtttttatgtgatttaggaccgtataaacgaaatccttgttcaccataatctctctcctagacatcctaccagttgtgatagaagcgtcaaagagagggacgataaccatgtctctcactttcgggaaccaaaagtttaacgcagcgttatccttgaagatggtcctctcggcattaagcattgctaacgtctttttgtagtgccacgcatcgtccatagccttgtaaaactcttcgcaatccacattattatggtacacaccacggaaaagagtaaaagtatccactctcgtaaacttacagtaccaagtgttgactctagtaactaaaaactccttatgatacacaaaccgttgactagcaggaaagaacgttttacacacatacttaattatattactaaaactgtgggtgtaattgagagtgctctcattatgaaagaaaaaacttaaattatcaccggacttctgaaaagtagctccaatctcatcgagtgtgactgtatcacaatctagaagcatattctcatggaaatgaaaggccgcgaaacaagttttcacattcttcctgagtagcgcagaaccgaactcctcaacagggatatcataaatgctatggagagctacagcgtaggtgtcagtgccatacgctgtcgtcaattcacactgttggaaaggtttgtcgcaatggacgaagtgcgggttctcagcgtagttgttgaaagctgccctctggtattcaggcacaggacgctgctgccttttcaaacgattcacataactgtaaatagcttccttgtgtccttcatggcgagcaatgtcacgtacatccagattaggcatgcagcagtgaacgtaatcgcgccctttgaaaaggtgcgcggaaaagttaccgccgatgtcgtacgtcagagaaccgaacggaacttgcatcatgagatactccaactcaagtgaccgaaggcctccggccaaggagtgcacagcggattgcgtatgagtaaaggaaatctcgaactccggatatgcgtttgttgcaatcagtgtctgttccgtagacactgccttggagaaatgaaccttgggacgtctggaacgagcattcagctcctcgactgcattatcgtaaacgcgacgagatgccaaatcattcaccaagctgttgcgtcccgcagcggcttggagagtttgcatgtcaattgtttgttgaaattgtgccatgttgttgttgtttgtattttgtttgttgttattgtaatttgttgttgttgttgcaataaaactaaaacagtactccaaatgaaatgaacttccttatatagatgttcagctatgcggggatgcgatcctggggtaggagagcacgtacggggccccgctcgtttgtggctctccgtgcggacatcccgtgcggacagtaccagaaagtgctctgtctctgctctatacggctctatacgcgtacctcttgaacggtgcgtggagaggagtggtgtgtcaatttccgccccgccctcgtgcggttccgcatgcatccaatctccttacgaattcccatggggagactagaattcgagctcggagaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgctattggatgcatgcggaaccgcacgagggcggggcggaaattgacacaccactcctctccacgcaccgttcaagaggtacgcgtatagagccgtatagagcagagacagagcactttctggtactgtccgcacgggatgtccgcacggagagccacaaacgagcggggccccgtacgtgctctcctaccccaggatcgcatccccgcatagctgaacatctatataaggaagttcatttcatttggagtactaggacgacctcgagtataagagctcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagctgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtgtggttgacgaattcgtcgattcggttgcagcattcaaggcggtcgacaacttcaggaagaggaagaagaaggttgaagagagggatgtagtcagcaagtacaagtacagaccggagaagtacgccggtcctgattcgttcaacctgaaggaggaaaacgtcctccagcactacaagcccgaataagcttggtcctgcaacttgaggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtagtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctcagagaggatgcacatgtgaccgagggacacgaagtggtttaaactatcagtgtttgacaggatatattggcgggtaaacctaagagaaaagagcgtttattagaataatcggatatttaaaagggcgtgaaaaggtttatccgttcgtccatttgtatgtgccaacca